(19) United States
(12) Patent Application Publication
(10) Pub. No.: US 2023/0355467 A1
(43) Pub. Date: Nov. 9, 2023

(54) Systems and Methods for Transition Time Reporting

(71) Applicant: ZOLL Medical Corporation, Chelmsford, MA (US)

(72) Inventors: Gregory R. Norton, Denver, CO (US); Nicole M. Spahn, Arvada, CO (US); James R. Fetterolf, Arvada, CO (US); Brian M. Smith, Arvada, CO (US)

(21) Appl. No.: 18/313,645

(22) Filed: May 8, 2023

Related U.S. Application Data
(60) Provisional application No. 63/339,477, filed on May 8, 2022.

(51) Int. Cl. A61H 31/00; G16H 40/67; G16H 15/00

(52) U.S. Cl. CPC A61H 31/005; A61H 31/007; A61H 31/006; G16H 40/67; G16H 15/00; A61N 1/39044

(57) ABSTRACT

A system for monitoring and/or reviewing transitions between types of medical treatment events provided for a patient during a rescue effort includes a medical device and a computing device. The medical device includes a chest compression sensor configured to receive time-correlated signals representative of chest compressions. The medical device is configured to generate a case file for the rescue effort comprising times of occurrence for a plurality of medical events. The computing device is configured to: receive the case file for the rescue effort, select and determine the time of occurrence for a first event of the plurality of medical events from the case file, select and determine the time of occurrence for a second event of the plurality of medical events, and determine a transition time between the time of occurrence of the first event and the time of occurrence of the second event.

### CROSS REFERENCE TO RELATED APPLICATION

This application claims priority to U.S. Provisional Patent Application No. 63/339,477 filed May 8, 2022, and entitled “Systems and Methods for Transition Time Reporting,” the disclosure of which is hereby incorporated by reference in its entirety.

### TECHNOLOGICAL FIELD

The present disclosure is related to systems and methods for determining and reporting transition times between events occurring during a rescue effort.

### BACKGROUND

Acute care is delivered to patients in emergency situations in the pre-hospital and hospital settings for patients experiencing a variety of acute medical conditions involving the timely diagnosis and treatment of disease states that, left alone, will likely degenerate into a life-threatening condition and, potentially, death. Stroke, dyspnea (difficulty breathing), traumatic arrest, myocardial infarction, and cardiac arrest are a few examples of disease states for which acute care is delivered to patients in an emergency setting. Acute care comprises different treatment and/or diagnosis, depending upon the disease state.

For cardiac arrest patients, cardiopulmonary resuscitation (CPR) may include a variety of therapeutic interventions including chest compressions, defibrillation, and ventilation. The first five to eight minutes of CPR, including chest compressions, can be critically important, largely because chest compressions help maintain blood circulation through the body and in the heart itself. The chest compressions may be performed by automated mechanical devices, such as, for example, the ZOLL® AutoPulse® mechanical chest compression device.

Alternatively, or additionally, chest compressions may be performed manually. During manual chest compressions, a rescuer, such as an acute care provider or lay person, places his or her hands on the patient's chest and pushes on the chest to perform the chest compression. Various devices are available for providing mechanical assistance for manual chest compressions. For example, an acute care provider may use a chest compression feedback device (e.g., CPR “puck”), such as those that incorporate REAL CPR HELP® technology, provided by ZOLL®, which provide real-time feedback to assist a caregiver in providing manual chest compressions according to target compression depth and rate. Other hand-held devices may be used, such as, for example, the ZOLL® ResQPump® active compression decompression device, positioned on the patient's chest to enhance movement of the patient's chest during chest compression and decompression. Ventilation is also a key part of CPR because ventilations help to provide much needed gas exchange (e.g., oxygen supply and carbon dioxide deposit) for the circulating blood.

CPR may be performed by a team of one or more acute care providers, for example, an emergency medical services (EMS) team made up of emergency medical technicians (EMTs), a hospital team including medical caregivers (e.g., doctors, nurses, etc.), and/or bystanders responding to an emergency event. In some instances, one acute care provider can provide chest compressions to the patient while another can provide ventilations to the patient, where the chest compressions and ventilations may be time and/or coordinated according to an appropriate CPR protocol. When professionals such as EMTs provide care, ventilation may be provided via a ventilation bag that an acute care provider squeezes, for example, rather than by mouth-to-mouth. CPR can be performed in conjunction with electrical shocks to the patient provided by an external defibrillator, such as an automatic external defibrillator (AED).

### SUMMARY

According to an aspect of the present disclosure, a system for monitoring and/or reviewing transitions between types of medical treatment events provided for a patient during a rescue effort includes at least one medical device and at least one computing device. The at least one medical device includes at least one chest compression sensor configured to receive time-correlated signals representative of chest compressions performed for the patient. The at least one medical device is configured to generate a case file for the rescue effort comprising times of occurrence for a plurality of medical events. The at least one processor is communicatively coupled with the at least one medical device. The at least one computing device is configured to: receive the case file for the rescue effort from the at least one medical device, select and determine the time of occurrence for at least one first event of the plurality of medical events from the case file, select and determine the time of occurrence for at least one second event of the plurality of medical events from the case file occurring after the selected at least one first event, determine a transition time between the time of occurrence of the at least one first event and the time of occurrence of the at least one second event, and generate a report that provides a transition time indication representative of the determined transition time for user review.

According to another aspect of the present disclosure, a computer-implemented method for providing transition times between types of medical treatment events provided for a patient includes a step of receiving a case file including a time-stamped record of a plurality of events occurring during a rescue effort generated based on analysis of motion signals generated by at least one chest compression sensor. The method also includes steps of: selecting and determining a time of occurrence of at least one first event of the plurality of events from the time-stamped record; selecting and determining a time of occurrence of at least one second event of the plurality of events from the time-stamped record occurring after the selected at least one first event; determining a transition time between the time of occurrence of the at least one first event and the time of occurrence of the at least one second event determined from the received time-stamped record; and generating a visual summary for the rescue effort comprising at least one transition time indication representative of the determined transition time.

According to another aspect of the disclosure, a system for monitoring and/or reviewing transitions between types of medical treatment events provided for a patient includes at least one medical device having at least one chest compression sensor configured to receive time-correlated signals representative of chest compressions performed for the patient; a visual display for providing information about the chest compressions performed for the patient; and at least one processor communicatively coupled to the at least one chest compression sensor and to the visual display. The at least one processor is configured to: receive and process the time-correlated signals from the at least one chest compression sensor, identify and determine a time of occurrence for at least one first event represented in the time-correlated signals, identify and determine a time of occurrence for at least one second event represented in the time-correlated signals occurring after the at least one first event, determine at least one transition time between the time of occurrence of the at least one first event and the time of occurrence of the at least one second event, and cause a transition time indication representative of the determined transition time to be displayed on the visual display.

According to another aspect of the disclosure, a system for monitoring and/or reviewing transitions between types of medical treatment events provided to a patient includes at least one medical device and at least one computing device. The at least one medical device includes at least one airflow path configured to be in fluid communication with an airway of a patient for providing manual or mechanical ventilations to the patient. The at least one airflow path includes at least one airflow sensor positioned to sense time-correlated signals representative of airflow in the patient's airway. The at least one medical device is configured to generate a case file for the rescue effort including times of occurrence for a plurality of medical events. The at least one computing device includes at least one processor communicatively coupled with the at least one patient ventilation unit. The at least one computing device is configured to: receive the case file for the rescue effort from the at least one medical device; select and determine the time of occurrence for at least one first event of the plurality of medical events from the case file; select and determine the time of occurrence for at least one second event of the plurality of medical events occurring after the selected at least one first event from the case file; determine a transition time between the time of occurrence of the at least one first event and the time of occurrence of the at least one second event; and generate a report that provides a transition time indication representative of the determined transition time for user review.

According to another aspect of the disclosure, a system for reviewing transitions between chest compressions performed by rescuers during a rescue event includes at least one chest compression sensor configured to receive time-correlated compression signals representative of chest compressions performed for the patient. The system also includes at least one first motion sensor configured to detect time-correlated movement signals representative of movement of hands or wrists of a first rescuer and at least one second motion sensor configured to detect time-correlated movement signals representative of movement of hands or wrists of a second rescuer. The system also includes at least one computing device having at least one processor communicatively coupled with the at least one chest compression sensor and with the first and second motion sensors. The at least one computing device is configured to: receive and process the time-correlated compression signals from the at least one chest compression sensor, receive and process the time-correlated movement signals from the first and second motion sensors, analyze the time-correlated compression signals and the time-correlated movement signals to identify portions of the compression signals for chest compressions by the first rescuer and portions of the compression signals for chest compressions by the second rescuer, identify and determine a time of occurrence for at least one first event occurring during the identified portions of the compression signals for chest compressions by the first rescuer, identify and determine a time of occurrence for at least one second event occurring during the identified portions of the compression signals for chest compressions by the second rescuer, determine a transition time between the time of occurrence of the at least one first event and the time of occurrence of the at least one second event, and generate a report that provides a transition time indication representative of the determined transition time for user review.

According to another aspect of the disclosure, a system for monitoring a transition time between medical treatment events includes a patient monitor and at least one computing device. The patient monitor includes a plurality of electrocardiogram (ECG) electrodes configured to be attached to a cardiothoracic region of a patient for receiving electrocardiogram signals, a user interface for providing information about treatment for the patient, and a processor in communication with the ECG electrodes and with the user interface. The processor is configured to receive and process the ECG signals, detect and record a time of occurrence of a heart attack event based on analysis of the ECG signals, cause a visual and/or audio notification about the heart attack event to be provided indicating detection of the heart attack event, and receive and record at least one time of occurrence for at least one post-heart attack event user input entered via the user interface. The at least one computing device includes at least one processor communicatively coupled with the patient monitor. The at least one computing device is configured to: receive the recorded time of occurrence for detection of the heart attack event and the recorded time of occurrence for the post-heart attack event user input, determine a transition time between the time of occurrence of the heart attack event and the time of occurrence of the post-heart attack event user input, and generate a report that provides an indication representative of the determined transition time.

According to another aspect of the disclosure, a system for reporting transition time trends in patient care data includes a computing device with at least one processor. The computing device is configured to receive and process a plurality of time-correlated signals generated by at least one resuscitation activity sensor during a plurality of different rescue efforts. Each of the plurality of signals is representative of at least one resuscitation activity performed for a patient during one of the rescue efforts. The computing device is further configured to: for each received and processed signal, analyze the signal to identify and determine a time of occurrence for at least one first event occurring during a particular rescue effort of the plurality of different rescue efforts; for each received and processed signal, analyze the processed signal to identify and determine a time of occurrence for at least one second event occurring during the particular rescue effort; for each received and processed signal, determine a transition time between the at least one first event and the at least one second event for each of the plurality of received and processed signals; and generate a report that provides a transition time indication representative of the determined transition time for each received and processed signal for user review.

According to another aspect of the disclosure, a patient ventilation monitoring system includes at least one chest compression sensor configured to receive time-correlated signals representative of chest compressions performed for the patient. The system further includes a patient ventilation unit having at least one airflow path configured to be in fluid communication with an airway of the patient for providing ventilations to the patient. The at least one airflow path includes at least one airflow sensor positioned to sense time-correlated signals representative of airflow in the patient's airway. The system further includes a visual display for providing information about the chest compressions and ventilations performed for the patient and at least one processor in communication with the at least one chest compression sensor, the at least one airflow sensor, and the visual display. The at least one processor is configured to: receive and process time-correlated signals from the at least one chest compression sensor to identify times of occurrence for the chest compressions; initiate an idle timer when a pause in chest compressions is detected in the processed time-correlated signals; cause a visual indication of the idle timer to be displayed on the visual display; receive and process time-correlated signals from the at least one airflow sensor; initiate a ventilation idle timer when a pause in ventilations is detected; and cause a notification or alarm to be provided on the visual display when the pause in ventilations is longer than a predetermined acceptable ventilation interval.

According to another aspect of the disclosure, a resuscitation activity monitoring and real-time feedback system includes at least one resuscitation activity sensor configured to receive signals representative of a resuscitation activity performed for a patient by a rescuer; a feedback device comprising a visual display; and at least one processor in communication with the at least one resuscitation activity sensor and the feedback device. The at least one processor is configured to: receive and process the signals from the at least one resuscitation activity sensor; analyze the processed signals to identify at least one first event; upon detection of the at least one first event, initiate a timer to monitor an elapsed time from occurrence of the at least one first event; and cause an alarm or notification to be displayed on the display of the feedback device when the elapsed time from the occurrence of the at least one first event exceeds a predetermined value.

Examples of the present disclosure will now be described in the following numbered clauses:

Clause 1: A system for monitoring and/or reviewing transitions between types of medical treatment events provided for a patient during a rescue effort, the system comprising: at least one medical device comprising at least one chest compression sensor configured to receive time-correlated signals representative of chest compressions performed for the patient, wherein the at least one medical device is configured to generate a case file for the rescue effort comprising times of occurrence for a plurality of medical events; and at least one computing device having at least one processor communicatively coupled with the at least one medical device, the at least one computing device configured to: receive the case file for the rescue effort from the at least one medical device, select and determine the time of occurrence for at least one first event of the plurality of medical events from the case file, select and determine the time of occurrence for at least one second event of the plurality of medical events from the case file occurring after the selected at least one first event, determine a transition time between the time of occurrence of the at least one first event and the time of occurrence of the at least one second event, and generate a report that provides a transition time indication representative of the determined transition time for user review.

Clause 2: The system of clause 1, wherein the at least one transition time is between at least one of: (i) turning on the at least one medical device and a start of manual chest compressions, (ii) turning on the at least one medical device and an end of manual chest compressions, (iii) turning on the at least medical device and a start of automated chest compressions, (iv) turning on the at least one medical device and an end of automated chest compressions, (v) the start of the manual chest compressions and the end of the manual chest compressions, (vi) the start of the manual chest compressions and the start of the automated chest compressions, (vii) the start of the manual chest compressions and the end of the automated chest compressions, (viii) the end of manual chest compressions and the start of automated chest compressions, (ix) the end of manual chest compressions and the end of automated chest compressions, or (x) the start of automated chest compressions and the end of automated chest compressions.

Clause 3: The system of clause 1 or clause 2, wherein the at least one medical device comprises a patient monitor comprising at least one patient physiological sensor configured to detect signals representative of at least one patient vital sign.

Clause 4: The system of clause 3, wherein the at least one patient vital sign comprises at least one of patient blood oxygen level, patient blood pressure, patient oxygen saturation (SPO2), patient end-tidal CO2, or patient heart rate.

Clause 5: The system of clause 3 or clause 4, wherein the at least one patient physiological sensor comprises at least one electrocardiogram (ECG) sensor.

Clause 6: The system of clause 5, wherein the at least one medical device is configured to monitor signals detected by the at least one ECG sensor to identify at least one of a return to spontaneous circulation (ROSC), a cardiac arrest event, or a heart attack event in the ECG signals, and wherein the generated case file further comprises information about the ROSC, the cardiac arrest event, or the heart attack event.

Clause 7: The system of clause 6, wherein the report generated by the at least one computing device comprises the information about the ROSC, the cardiac arrest event, or the heart attack event provided by the at least one medical device.

Clause 8: The system of any of clauses 5-7, wherein the at least one medical device comprises a defibrillator comprising at least one therapeutic electrode for providing cardiac therapy for the patient based on an analysis of the signals detected by the at least one ECG sensor.

Clause 9: The system of any of clauses 1-8, wherein the at least one chest compression sensor comprises at least one of an accelerometer, velocity sensor, force sensor, or impedance sensor.

Clause 10: The system of any of clauses 1-9, wherein the at least one chest compression sensor comprises a single axis or a multi-axis accelerometer, and wherein the accelerometer is configured to be positioned on a sternum of the patient.

Clause 11: The system of claim 10, further comprising a housing configured to be positioned on the sternum of the patient between hands of a rescuer performing the chest compressions and a chest of the patient, wherein the accelerometer is positioned in the housing.

Clause 12: The system of any of clauses 1-11, wherein, to generate the case file, the at least one medical device is configured to: receive and process the time-correlated signals from the at least one chest compression sensor, identify and determine the times of occurrence for the plurality of the medical events represented in the time-correlated signals, and generate the case file for the rescue effort comprising the times of occurrence for the plurality of medical events represented in the time-correlated signals.

Clause 13: The system of clause 12, wherein the at least one first event comprises an end of manual chest compressions, and the at least one second event comprises a start of automated chest compressions.

Clause 14: The system of clause 13, wherein the at least one medical device is configured to identify and determine the time of occurrence for the end of the manual chest compressions by: generating at least one compression waveform from the received and processed time-correlated signals; identifying portions of the at least one compression waveform representative of manual chest compressions provided for the patient; and determining a final time of the portions of the at least one compression waveform representative of the manual chest compressions.

Clause 15: The system of clause 14, wherein the at least one medical device is configured to identify and determine the time of occurrence for the start of the automated chest compressions by: identifying portions of the at least one compression waveform representative of automated chest compressions provided for the patient; and determining a first time of the portions of the at least one compression waveform representative of the automated chest compressions.

Clause 16: The system of clause 14 or clause 15, wherein the at least one medical device is configured to identify the portions of the at least one chest compression waveform representative of manual chest compressions by: calculating at least one chest compression parameter value for multiple segments of the at least one compression waveform; comparing the calculated at least one chest compression parameter value for the multiple segments to a target range for the at least one chest compression parameter values representative of manual chest compressions; and identifying segments of the multiple segments of the at least one compression waveform with the at least one chest compression parameter value within the target range.

Clause 17: The system of clause 16, wherein the at least one chest compression parameter value comprises at least one of compression rate, compression depth, compression hold time, variation in compression rate, variation in compression depth, variation in hold time, compression width, relaxation time, release time, compression average velocity, compression maximum velocity, or velocity minimum to maximum time (per chest compression cycle).

Clause 18: The system of any of clauses 12-17, wherein the at least one first event comprises turning on the at least one medical device, and wherein the time of occurrence for turning on the at least one medical device is a first time recorded in the time-correlated signals, and the at least one second event comprises a start of manual chest compressions, an end of the manual chest compressions, a start of automated chest compressions, or an end of the automated chest compressions.

Clause 19: The system of any of clauses 1-18, wherein the generated case file for the rescue effort comprises the time-correlated signals received by the at least one chest compression sensor, and wherein the at least one computing device is configured to process the time-correlated signals to identify and determine the times of occurrence for the plurality of the medical events represented in the time-correlated signals.

Clause 20: The system of any of clauses 1-19, wherein the at least one computing device further comprises a visual display, and wherein the at least one computing device is further configured to cause the transition time indication representative of the determined transition time to be displayed on the visual display.

Clause 21: The system of any of clauses 1-20, further comprising a chest compressor configured to be positioned on a chest of the patient for providing automated chest compressions for the patient.

Clause 22: The system of clause 21, wherein the chest compressor comprises a compression belt and a belt tensioner configured to tighten the compression belt around the chest of the patient in order to compress the chest of the patient.

Clause 23: The system of clause 21 or clause 22, wherein the chest compressor is a piston-based device comprising: a piston, a piston driver, support structures for supporting the piston and the piston driver, and a compression pad affixed to the piston.

Clause 24: The system of any of clauses 1-23, wherein the at least one computing device comprises a local portable computing device in wired or wireless communication with the at least one medical device.

Clause 25: The system of any of clauses 1-24, wherein the at least one computing device is integral with and/or a component of the at least one medical device, and is configured to cause the generated report to be displayed on a display of the at least one medical device.

Clause 26: The system of any of clauses 1-25, wherein the at least one computing device comprises a remote computing device or remote computer server configured to receive the case file for the rescue effort via a wired or wireless data transmission initiated from a communication device of the at least one medical device.

Clause 27: A computer-implemented method for providing transition times between types of medical treatment events provided for a patient, the method comprising: receiving a case file comprising a time-stamped record of a plurality of events occurring during a rescue effort generated based on analysis of motion signals generated by at least one chest compression sensor; selecting and determining a time of occurrence of at least one first event of the plurality of events from the time-stamped record; selecting and determining a time of occurrence of at least one second event of the plurality of events from the time-stamped record occurring after the selected at least one first event; determining a transition time between the time of occurrence of the at least one first event and the time of occurrence of the at least one second event determined from the received time-stamped record; and generating a visual summary for the rescue effort comprising at least one transition time indication representative of the determined transition time.

Clause 28: The method of clause 27, wherein the at least one transition time is between at least one of: (i) turning on the at least one medical device and a start of manual chest compressions, (ii) turning on the at least one medical device and an end of manual chest compressions, (iii) turning on the at least medical device and a start of automated chest compressions, (iv) turning on the at least one medical device and an end of automated chest compressions, (v) the start of the manual chest compressions and the end of the manual chest compressions, (vi) the start of the manual chest compressions and the start of the automated chest compressions, (vii) the start of the manual chest compressions and the end of the automated chest compressions, (viii) the end of manual chest compressions and the start of automated chest compressions, (ix) the end of manual chest compressions and the end of automated chest compressions, or (x) the start of automated chest compressions and the end of automated chest compressions.

Clause 29: The method of clause 27 or clause 28, further comprising receiving information from at least one patient physiological sensor configured to detect signals representative of at least one patient vital sign, wherein the visual summary further comprises at least one visual indication representative of the at least one patient vital sign.

Clause 30: The method of clause 29, wherein the at least one patient vital sign comprises at least one of patient blood oxygen level, patient blood pressure, patient oxygen saturation (SPO2), patient end-tidal CO2, or patient heart rate.

Clause 31: The method of any of clauses 27-30, further comprising receiving information about a return to spontaneous circulation (ROSC), a cardiac arrest event, or a heart attack event determined by monitoring ECG signals of the patient, wherein the visual summary further comprises at least one visual indication indicating occurrence of the ROSC, the cardiac arrest event, or the heart attack event.

Clause 32: The method of any of clauses 27-31, wherein the at least one first event comprises an end of manual chest compressions, and the at least one second event comprises a start of automated chest compressions.

Clause 33: The method of any of clauses 27-32, further comprising making the visual summary available for download via a computer network, such that the visual summary is viewable by a remote computer device.

Clause 34: A system for monitoring and/or reviewing transitions between types of medical treatment events provided for a patient, the system comprising: at least one medical device comprising at least one chest compression sensor configured to receive time-correlated signals representative of chest compressions performed for the patient; a visual display for providing information about the chest compressions performed for the patient; and at least one processor communicatively coupled to the at least one chest compression sensor and to the visual display, wherein the at least one processor is configured to: receive and process the time-correlated signals from the at least one chest compression sensor, identify and determine a time of occurrence for at least one first event represented in the time-correlated signals, identify and determine a time of occurrence for at least one second event represented in the time-correlated signals occurring after the at least one first event, determine at least one transition time between the time of occurrence of the at least one first event and the time of occurrence of the at least one second event, and cause a transition time indication representative of the determined transition time to be displayed on the visual display.

Clause 35: The system of clause 34, wherein the least one transition time is between at least one of: (i) turning on the at least one medical device and a start of manual chest compressions, (ii) turning on the at least one medical device and an end of manual chest compressions, (iii) turning on the at least medical device and a start of automated chest compressions, (iv) turning on the at least one medical device and an end of automated chest compressions, (v) the start of the manual chest compressions and the end of the manual chest compressions, (vi) the start of the manual chest compressions and the start of the automated chest compressions, (vii) the start of the manual chest compressions and the end of the automated chest compressions, (viii) the end of manual chest compressions and the start of automated chest compressions, (ix) the end of manual chest compressions and the end of automated chest compressions, or (x) the start of automated chest compressions and the end of automated chest compressions.

Clause 36: The system of clause 34 or clause 35, wherein the at least one medical device comprises a patient monitor, the patient monitor further comprising at least one patient physiological sensor configured to detect signals representative of at least one patient vital sign.

Clause 37: The system of clause 36, wherein the at least one patient vital sign comprises at least one of patient blood oxygen level, patient blood pressure, patient oxygen saturation (SPO2), patient end-tidal CO2, or patient heart rate.

Clause 38: The system of clause 36 or clause 37, wherein the at least one processor is configured to cause visual indications representative of the at least one patient vital sign to be displayed on the visual display along with the transition time indication.

Clause 39: The system of any of clauses 36-38, wherein the at least one patient physiological sensor comprises an electrocardiogram (ECG) sensor.

Clause 40: The system of clause 39, wherein the at least one processor is configured to: monitor signals detected by the at least one electrocardiogram (ECG) sensor to identify at least one of a return to spontaneous circulation (ROSC) or a cardiac arrest event in the ECG signals; and cause information about the ROSC or the cardiac arrest event to be displayed on the visual display along with the transition time indication.

Clause 41: The system of clause 39 or clause 40, wherein the at least one medical device comprises a defibrillator comprising at least one therapeutic electrode for providing cardiac therapy for the patient based on an analysis of the signals detected by the at least one ECG sensor, and wherein the at least one processor comprises a processor of the at least one medical device, which is further configured to control the defibrillator to provide the cardiac therapy to the patient.

Clause 42: The system of any of clauses 34-41, wherein the at least one chest compression sensor comprises a single axis or a multi-axis accelerometer.

Clause 43: The system of clause 42, wherein the accelerometer is configured to be positioned on a sternum of the patient.

Clause 44: The system of clause 42 or clause 43, further comprising a housing configured to be positioned on a sternum of the patient between hands of a rescuer performing the chest compressions and a chest of the patient, wherein the accelerometer is positioned in the housing.

Clause 45: The system of any of clauses 34-44, wherein the at least one first event comprises an end of manual chest compressions, and the at least one second event comprises a start of automated chest compressions.

Clause 46: The system of clause 45, wherein the at least one processor is configured to identify and determine the time of occurrence for the end of the manual chest compressions by: generating at least one compression waveform from the received and processed time-correlated signals; identifying portions of the at least one compression waveform representative of manual chest compressions provided for the patient; and determining the latest time represented by the portions of the at least one compression waveform representative of manual chest compressions.

Clause 47: The system of clause 46, wherein the at least one processor is configured to identify and determine the time of occurrence for the start of the automated chest compressions by: identifying portions of the at least one compression waveform representative of automated chest compressions provided for the patient; and determining an earliest time represented by the portions of the at least one compression waveform representative of the automated chest compressions.

Clause 48: The system of clause 46 or clause 47, wherein the at least one processor is configured to identify the portions of the at least one chest compression waveform representative of manual chest compressions by: calculating at least one chest compression parameter value for multiple segments of the at least one compression waveform; comparing the calculated at least one chest compression parameter value for the multiple segments to a target range of parameter values representative of manual chest compressions; and identifying segments of the multiple segments of the at least one compression waveform with the at least one chest compression parameter value within the target range for the manual chest compression.

Clause 49: The system of clause 48, wherein the at least one chest compression parameter value comprises at least one of variation of compression rate, compression depth, hold time, variation in compression rate, variation in compression depth, variation in hold time, compression width, relaxation time, release time, compression velocity, velocity amplitude, or velocity minimum to maximum time (per chest compression cycle).

Clause 50: The system of any of clauses 34-49, wherein the at least one first event comprises turning on the at least one medical device, and wherein the time of occurrence for turning on the at least one medical device is an earliest time recorded in the time-correlated signals, and the at least one second event comprises a start of manual compressions, an end of manual compressions, the start of automated compressions, or an end of automated compressions.

Clause 51: The system of any of clauses 34-41, wherein the at least one visual display comprises a portable electronic device comprising at least one of a cellular telephone, smartphone, personal digital assistant, or a computer tablet.

Clause 52: The system of clause 51, wherein the transition time indicator is provided on the visual display of the at least one computing device in real-time during a rescue effort.

Clause 53: The system of clause 51 or clause 52, wherein the at least one processor is further configured to cause an alarm or notification to be provided on the visual display instructing a rescuer to begin automated chest compressions after manual chest compressions have been performed for longer than a predetermined manual compression duration.

Clause 54: The system of any of clauses 34-53, wherein the at least one processor is further configured to analyze the time correlated signals to determine at least one chest compression parameter, the at least one chest compression parameter comprising at least one of an average chest compression depth, an average chest compression rate, a chest compression fraction, or pre-shock delay, or post-shock delay and to cause an indication representative of the determined at least one chest compression parameter to be displayed on the at least one visual display in proximity to the a transition time indication.

Clause 55: The system of any of clauses 34-54, further comprising a chest compressor configured to be positioned on a chest of the patient for providing the automated chest compressions for the patient.

Clause 56: The system of clause 55, wherein the chest compressor comprises a compression belt and a belt tensioner configured to tighten the compression belt around the chest of the patient in order to compress the chest of the patient.

Clause 57: The system of clause 55 or clause 56, wherein the chest compressor is a piston-based device comprising: a piston, a piston driver, support structures for supporting the piston and the piston driver, and a compression pad affixed to the piston.

Clause 58: A system for monitoring and/or reviewing transitions between types of medical treatment events provided to a patient, comprising: at least one medical device comprising at least one airflow path configured to be in fluid communication with an airway of a patient for providing manual or mechanical ventilations to the patient, the at least one airflow path comprising at least one airflow sensor positioned to sense time-correlated signals representative of airflow in the patient's airway, wherein the at least one medical device is configured to generate a case file for the rescue effort comprising times of occurrence for a plurality of medical events; and at least one computing device having at least one processor communicatively coupled with the at least one patient ventilation unit, the at least one computing device configured to: receive the case file for the rescue effort from the at least one medical device; select and determine the time of occurrence for at least one first event of the plurality of medical events from the case file; select and determine the time of occurrence for at least one second event of the plurality of medical events occurring after the selected at least one first event from the case file; determine a transition time between the time of occurrence of the at least one first event and the time of occurrence of the at least one second event; and generate a report that provides a transition time indication representative of the determined transition time for user review.

Clause 59: The system of clause 58, wherein the at least one transition time is between at least one of: (i) activation of the at least one medical device and a start of manual ventilations, (ii) activation of the at least one medical device and an end of the manual ventilations, (iii) activation of the at least one medical device and a start of the mechanical ventilations, (iv) activation of the at least one medical device and an end of the mechanical ventilations, (v) the start of the manual ventilations and the end of the manual ventilations, (vi) the start of the manual ventilations and the start of the mechanical ventilations, (vii) the start of the manual ventilations and the end of the mechanical ventilations, (viii) the end of the manual ventilations and the start of the mechanical ventilations, (ix) the end of the manual ventilations and the end of the mechanical ventilations; and (x) the start of the mechanical ventilations and the end of the mechanical ventilations.

Clause 60: The system of clause 58 or clause 59, wherein the at least one medical device comprises a mechanical ventilator configured to be connected to the airflow path to provide the mechanical ventilations to the patient.

Clause 61: The system of clause 60, wherein the airflow path comprises at least one of an intubation tube or a mask that seals to and fits over a lower portion of a face of the patient for providing airflow to the patient.

Clause 62: The system of clause 61, wherein the at least one medical device further comprises a flexible bag configured to be connected to the airflow path to provide manual ventilations for the patient.

Clause 63: The system of any of clauses 58-62, further comprising at least one capnography sensor configured to detect data representative of CO2 from an exhaled breath of the patient.

Clause 64: The system of any of clauses 58-63, wherein the at least one medical device is further configured to determine a ventilation rate for the patient based on analysis of the time-correlated signals from the at least one airflow sensor.

Clause 65: The system of clause 64, wherein the at least one medical device is configured to compare the determined ventilation rate to a target ventilation rate range and cause a ventilation rate indication to be displayed on a visual display of the at least one medical device indicating whether the ventilation rate is within or outside of the target range.

Clause 66: The system of clause 65, wherein the ventilation rate range comprises a ventilation rate of about 10 ventilations per minute to about 20 ventilations per minute.

Clause 67: The system of any of clauses 58-66, wherein the at least one first event comprises activation of the at least one medical device, and the at least one second event comprises a start of manual ventilations, an end of manual ventilations, a start of mechanical ventilations, or an end of mechanical ventilations.

Clause 68: The system of any of clauses 58-67, wherein, to generate the case file, the at least one medical device is configured to: receive and process the time-correlated signals from the at least one airflow sensor, identify and determine the times of occurrence for the plurality of medical events represented in the time-correlated signals, and generate the case file for the rescue effort comprising the times of occurrence for the plurality of medical events.

Clause 69: The system of clause 68, wherein the at least one first event comprises an end of manual ventilations and the at least one second event comprises a start of mechanical ventilations.

Clause 70: The system of clause 69, wherein the at least one medical device is configured to identify and determine the time of occurrence for the end of the manual ventilations by: generating at least one ventilation waveform from the received and processed time-correlated signals; identifying portions of the at least one ventilation waveform representative of manual ventilations provided for the patient; and determining the latest time represented by the portions of the at least one ventilation waveform representative of the manual ventilations.

Clause 71: The system of clause 70, wherein the at least one medical device is configured to identify and determine the time of occurrence for the start of the mechanical ventilations by: identifying portions of the at least one ventilation waveform representative of mechanical ventilations provided for the patient; and determining an earliest time represented by the portions of the at least one ventilation waveform representative of the mechanical ventilations.

Clause 72: The system of any of clauses 58-71, wherein the at least one medical device comprises a first airflow sensor configured to sense airflow generated by manual ventilations and a second airflow sensor configured to sense airflow generated by a mechanical ventilator, and wherein the at least one medical device is configured to receive and process signals from the first airflow sensor and from the second airflow sensor.

Clause 73: The system of clause 72, wherein the at least one medical device is configured to distinguish between manual ventilations and mechanical ventilations in the received time-correlated signals based on whether the signals received from the first airflow sensor or the second airflow sensor.

Clause 74: The system of clause 72 or clause 73, wherein the at least one first event comprises an end of manual ventilations identified based on a latest time of the time-correlated signals received from the first airflow sensor, and the at least one second event comprises a start of mechanical ventilations identified based on an earliest time of the time correlated signal received from the second airflow sensor.

Clause 75: The system of any of clauses 58-74, wherein the at least one computing device comprises a visual display for providing information about the ventilations performed for the patient, and wherein the at least one computing device is configured to cause a transition time indication representative of the determined transition time to be displayed on the visual display.

Clause 76: The system of any of clauses 58-75, wherein the at least one computing device comprises a portable computing device in wireless communication with the at least one medical device.

Clause 77: A system for reviewing transitions between chest compressions performed by rescuers during a rescue event, the system comprising: at least one chest compression sensor configured to receive time-correlated compression signals representative of chest compressions performed for the patient; at least one first motion sensor configured to detect time-correlated movement signals representative of movement of hands or wrists of a first rescuer; at least one second motion sensor configured to detect time-correlated movement signals representative of movement of hands or wrists of a second rescuer; at least one computing device having at least one processor communicatively coupled with the at least one chest compression sensor and with the first and second motion sensors, wherein the at least one computing device configured to: receive and process the time-correlated compression signals from the at least one chest compression sensor, receive and process the time-correlated movement signals from the first and second motion sensors, analyze the time-correlated compression signals and the time-correlated movement signals to identify portions of the compression signals for chest compressions by the first rescuer and portions of the compression signals for chest compressions by the second rescuer, identify and determine a time of occurrence for at least one first event occurring during the identified portions of the compression signals for chest compressions by the first rescuer, identify and determine a time of occurrence for at least one second event occurring during the identified portions of the compression signals for chest compressions by the second rescuer, determine a transition time between the time of occurrence of the at least one first event and the time of occurrence of the at least one second event, and generate a report that provides a transition time indication representative of the determined transition time for user review.

Clause 78: The system of clause 77, wherein the least one transition time is between at least one of: (i) a start of chest compressions by the first rescuer and an end of chest compressions by the first rescuer, (ii) the start of chest compressions by the first rescuer and a start of chest compressions by the second rescuer, (iii) the start of chest compressions by the first rescuer and an end of chest compressions by the second rescuer, (iv) the end of chest compressions by the first rescuer and the start of chest compressions by the second rescuer, (v) the end of chest compressions by the first rescuer and the end of chest compressions by the second rescuer, (vi) the start of chest compressions by the second rescuer and the end of the chest compressions by the second rescuer, and (vii) the end of the chest compressions by the second rescuer and a restart of chest compression by the first rescuer.

Clause 79: The system of clause 77 or clause 78, wherein the at least one chest compression sensor comprises a single axis or a multi-axis accelerometer.

Clause 80: The system of clause 79, wherein the accelerometer is configured to be positioned on a sternum of the patient.

Clause 81: The system of clause 79 or clause 80, further comprising a housing configured to be positioned on the patient's sternum between hands of a rescuer performing the chest compressions and a chest of the patient, wherein the accelerometer is positioned in the housing.

Clause 82: The system of any of clauses 77-81, further comprising a first wrist-worn device configured to be worn by the first rescuer, which comprises the first motion sensor, and a second wrist-worn device configured to be worn by the second rescuer, which comprises the second motion sensor.

Clause 83: The system of any of clauses 77-82, wherein the at least one computing device is configured to analyze the time-correlated compression signals and the time-correlated movement signals by: determining at least one parameter value for multiple segments of the time-correlated compression signals; determining at least one parameter value for multiple segments of the time-correlated movement signals; comparing the at least one parameter value for multiple segments of the time-correlated compression signals to the at least one parameter value for the multiple segments of the time-correlated movement signals; and identifying segments of the at least one compression signals as first rescuer segments or second rescuer segments based on the comparison.

Clause 84: The system of clause 83, wherein a particular segment is identified as a first rescuer segment when the at least one parameter value for the particular segment of the time-correlated compression signals is within a predetermined amount of the at least one parameter value for the time-correlated movement signal for the first motion sensor.

Clause 85: The system of clause 83 or clause 84, wherein a particular segment is identified as a second rescuer segment when the at least one parameter value for the particular segment of the time-correlated compression signals is within a predetermined amount of the at least one parameter value for the time-correlated movement signal for the second motion sensor.

Clause 86: The system of any of clauses 83-85, wherein the at least one parameter value comprises a value for at least one of displacement, velocity, or acceleration.

Clause 87: The system of any of clauses 77-86, wherein the at least one computing device comprises a visual display for providing information about the chest compressions performed for the patient, and wherein the at least one computing device is configured to cause the transition time indication representative of the determined transition time to be displayed on the visual display.

Clause 88: A system for monitoring a transition time between medical treatment events, comprising: a patient monitor comprising a plurality of electrocardiogram (ECG) electrodes configured to be attached to a cardiothoracic region of a patient for receiving electrocardiogram signals, a user interface for providing information about treatment for the patient, and a processor in communication with the ECG electrodes and with the user interface, wherein the processor is configured to receive and process the ECG signals, detect and record a time of occurrence of a heart attack event based on analysis of the ECG signals, cause a visual and/or audio notification about the heart attack event to be provided indicating detection of the heart attack event, and receive and record at least one time of occurrence for at least one post-heart attack event user input entered via the user interface; and at least one computing device having at least one processor communicatively coupled with the patient monitor, the at least one computing device configured to: receive the recorded time of occurrence for detection of the heart attack event and the recorded time of occurrence for the post-heart attack event user input, determine a transition time between the time of occurrence of the heart attack event and the time of occurrence of the post-heart attack event user input, and generate a report that provides an indication representative of the determined transition time.

Clause 89: The system of clause 88, wherein the post-heart attack event user input comprises an instruction to transmit a heart attack notification to a remote computing network or device.

Clause 90: The system of clause 89, wherein the patient monitor further comprises a wireless data transceiver, and wherein the at least one processor is further configured to cause the wireless data transceiver to transmit the time of occurrence of the detected heart attack event and the time of occurrence of the instruction to transmit the heart attack notification to the remote computing network or device via the wireless data transceiver.

Clause 91: The system of any of clauses 88-90, wherein the post-heart attack event user input comprises a confirmation that a treatment activity was performed for the patient.

Clause 92: The system of clause 91, wherein the treatment activity comprises an epinephrine injection.

Clause 93: The system of any of clauses 88-92, wherein the heart attack event comprises an ST-elevation myocardial infarction (STEMI).

Clause 94: The system of any of clauses 88-93, wherein the plurality of ECG electrodes are configured to obtain a 12-lead ECG.

Clause 95: The system of any of clauses 88-94, wherein the at least one computing device is further configured to receive information about treatment of the patient by a medical facility after the rescue event.

Clause 96: The system of clause 95, wherein the information about the treatment of the patient comprises an electronic patient health record.

Clause 97: The system of clause 95 or clause 96, wherein the information about the treatment of the patient comprises a drug administration time, a stent time, and/or a balloon time for the patient, and wherein the at least one processor is configured to display a transition time between the time of occurrence of the cardiac arrest event and the drug administration time, stent time, and/or balloon time on a visual display of the at least one computing device.

Clause 98: A system for reporting transition time trends in patient care data, the system comprising a computing device comprising at least one processor, wherein the computing device is configured to: receive and process a plurality of time-correlated signals generated by at least one resuscitation activity sensor during a plurality of different rescue efforts, wherein each of the plurality of signals is representative of at least one resuscitation activity performed for a patient during one of the rescue efforts; for each received and processed signal, analyze the signal to identify and determine a time of occurrence for at least one first event occurring during a particular rescue effort of the plurality of different rescue efforts; for each received and processed signal, analyze the processed signal to identify and determine a time of occurrence for at least one second event occurring during the particular rescue effort; for each received and processed signal, determine a transition time between the at least one first event and the at least one second event for each of the plurality of received and processed signals; and generate a report that provides a transition time indication representative of the determined transition time for each received and processed signal for user review.

Clause 99: The system of clause 98, wherein the at least one resuscitation activity sensor comprises a chest compression sensor and/or a ventilation airflow sensor.

Clause 100: The system of clause 98 or clause 99, wherein the least one transition time is between at least one of: (i) a start of the manual chest compressions and an end of the manual chest compressions, (ii) the start of the manual chest compressions and a start of the automated chest compressions, (iii) the start of the manual chest compressions and an end of the automated chest compressions, (iv) the end of manual chest compressions and the start of automated chest compressions, (vi) the end of manual chest compressions and the end of automated chest compressions, or (vii) the start of automated chest compressions and the end of automated chest compressions.

Clause 101: The system of any of clauses 98-100, wherein the at least one transition time is between at least one of: (i) a start of the manual ventilations and an end of the manual ventilations, (ii) the start of the manual ventilations and a start of the mechanical ventilations, (iii) the start of the manual ventilations and an end of the mechanical ventilations, (iv) the end of the manual ventilations and the start of the mechanical ventilations, (v) the end of the manual ventilations and the end of the mechanical ventilations; and (vi) the start of the mechanical ventilations and the end of the mechanical ventilations.

Clause 102: The system of any of clauses 98-101, wherein the report further comprises an average transition time for the plurality of received and processed signals determined based on the determined transition time between the at least one first event and the at least one second event for each of the plurality of received and processed signals.

Clause 103: A patient ventilation monitoring system, comprising: at least one chest compression sensor configured to receive time-correlated signals representative of chest compressions performed for the patient; a patient ventilation unit comprising at least one airflow path configured to be in fluid communication with an airway of the patient for providing ventilations to the patient, the at least one airflow path comprising at least one airflow sensor positioned to sense time-correlated signals representative of airflow in the patient's airway; a visual display for providing information about the chest compressions and ventilations performed for the patient; and at least one processor in communication with the at least one chest compression sensor, the at least one airflow sensor, and the visual display, wherein the at least one processor is configured to: receive and process time-correlated signals from the at least one chest compression sensor to identify times of occurrence for the chest compressions; initiate an idle timer when a pause in chest compressions is detected in the processed time-correlated signals; cause a visual indication of the idle timer to be displayed on the visual display; receive and process time-correlated signals from the at least one airflow sensor; initiate a ventilation idle timer when a pause in ventilations is detected; and cause a notification or alarm to be provided on the visual display when the pause in ventilations is longer than a predetermined acceptable ventilation interval.

Clause 104: The system of clause 103, wherein the at least one processor is further configured to analyze the received and processed signals for the chest compressions and provide feedback on the visual display for guiding a rescuer in performing chest compressions according to a predetermined CPR protocol.

Clause 105: The system of clause 103 or clause 104, wherein identifying the pause in chest compressions comprises monitoring an elapsed time since a most-recent chest compression and determining that there is a pause in chest compressions when the elapsed time exceeds a time permitted by a predetermined CPR protocol for the patient by at least predetermined amount.

Clause 106: The system of clause 102, wherein identifying the pause in ventilations comprises monitoring an elapsed time since a most-recent ventilation was provided to the patient and determining that there is a pause in ventilations when the elapsed time exceeds a time permitted by the predetermined CPR protocol by a predetermined amount.

Clause 107: The system of clause 105 or clause 106, wherein the CPR protocol comprises repeatedly performing 30 chest compressions followed by 2 ventilations.

Clause 108: A resuscitation activity monitoring and real-time feedback system comprising: at least one resuscitation activity sensor configured to receive signals representative of a resuscitation activity performed for a patient by a rescuer; a feedback device comprising a visual display; and at least one processor in communication with the at least one resuscitation activity sensor and the feedback device, wherein the at least one processor is configured to: receive and process the signals from the at least one resuscitation activity sensor; analyze the processed signals to identify at least one first event; upon detection of the at least one first event, initiate a timer to monitor an elapsed time from occurrence of the at least one first event; cause an alarm or notification to be displayed on the display of the feedback device when the elapsed time from the occurrence of the at least one first event exceeds a predetermined value.

Clause 109: The system of clause 108, wherein the at least one processor is further configured to cause a visual indication of the timer to be displayed on the visual display.

Clause 110: The system of clause 108 or clause 109, wherein the at least one processor is further configured to: monitor the received and processed signals in real-time to detect at least one second event; record a transition time comprising an elapsed time between the at least one first event and the at least one second event from the timer; and upon recording the transition time, modify the visual display of the feedback device to replace the visual indication representative of the timer with a visual indication representative of the recorded transition time between the at least one first event and the at least one second event.

Clause 111: The system of clause 110, wherein the at least one processor is further configured to compare the recorded transition time to a target transition time range and to modify an appearance of the visual indication representative of the determined transition time based on the comparison.

Clause 112: The system of clause 111, wherein the visual indication representative of the recorded transition time has a first appearance when the recorded transition time is within the target range and a second appearance when the recorded transition time is outside of the target range.

### BRIEF DESCRIPTION OF THE DRAWINGS

Various aspects of the disclosure are discussed below with reference to the accompanying figures, which are not intended to be drawn to scale. The figures are included to provide an illustration and a further understanding of various examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure. A quantity of each component in a particular figure is an example only and other quantities of each, or any, component could be used.

FIG. 1A is a schematic drawing of a system for reporting transition times between types of chest compressions, according to an aspect of the present disclosure;

FIG. 1B is a schematic drawing of another example of a system for reporting transition times between types of chest compressions, according to an aspect of the present disclosure;

FIG. 1C is a schematic drawing of a medical device configured for reporting transition times between different types of chest compressions, according to an aspect of the present disclosure;

FIG. 2A is a drawing of a rescue scene showing a rescuer using the chest compression sensor of FIGS. 1A-1C to provide manual chest compressions for a patient, according to an aspect of the present disclosure;

FIGS. 2B and 2C are drawings of examples of chest compressors for providing automated chest compressions for a patient, which can be used with the CPR transition time reporting systems of the present disclosure;

FIGS. 2D-2F are schematic drawings showing systems including embodiments of mechanical chest compressors and medical devices which can be configured for generating case files, according to aspects of the present disclosure;

FIG. 3A shows graphs for displacement, velocity, and acceleration derived from signals detected by a chest compression sensor during automated chest compressions;

FIG. 3B shows graphs for displacement, velocity, and acceleration derived from signals detected by a chest compression sensor during manual chest compressions;

FIG. 4A is a timeline showing displacement data from a case file for manual and automated chest compressions based on compression signals detected by a chest compression sensor, according to an aspect of the present disclosure;

FIG. 4B is an annotated timeline showing events occurring during a rescue effort generated based on data from a case file, according to an aspect of the present disclosure;

FIG. 4C is a timeline that shows times of occurrence for events occurring during a rescue effort, according to an aspect of the present disclosure;

FIG. 4D is a Transition Time Report showing transition times for events occurring during a rescue effort generated based on data from a case file, according to an aspect of the present disclosure;

FIG. 4E is another example of a report showing transition times generated based on data from a case file, according to an aspect of the present disclosure;

FIG. 4F is a drawing of a graphical user interface (GUI) including a CPR Transition Time Summary table generated from data from a case file, which can be provided on a user's computer allowing the user to review transition time information and other information about the rescue effort, according to an aspect of the present disclosure;

FIG. 4G is another example of a report showing transition times generated based on data from a case file, according to an aspect of the present disclosure;

FIG. 5A is a flow chart showing a method for generating a case file for a rescue effort, which can be performed by a medical device or another computing device at a rescue scene or remote from the rescue scene, according to an aspect of the present disclosure;

FIG. 5B is a flow chart showing a method for generating a transition time report from case file data, which can be performed by a computing device at a rescue scene or remote from a rescue scene, according to an aspect of the present disclosure;

FIG. 5C is a flow chart showing an embodiment of a method for processing a list of events to ensure that each compression start event is matched with a compression end event, according to an aspect of the present disclosure;

FIGS. 6A-8B are illustrative embodiments of user interface screens that can be displayed on a computing device for a user to review chest compression information from case files, according to an aspect of the present disclosure;

FIG. 9A is a schematic drawing of an embodiment of a system for monitoring and determining transition times between types of ventilations provided to a patient during a rescue effort, according to an aspect of the present disclosure;

FIG. 9B is a drawing of the system of FIG. 9A used for providing manual ventilations to a patient, according to aspects of the present disclosure;

FIG. 9C is a drawing of the system of FIG. 9A used for providing mechanical ventilations to a patient, according to aspects of the present disclosure;

FIG. 10A is a waveform generated from case file data showing airflow through a patient airway during manual ventilations detected by the first airflow sensor of FIG. 9A;

FIG. 10B is a waveform generated from case file data showing airflow through a patient airway during mechanical ventilations detected by the second airflow sensor of FIG. 9A;

FIG. 10C is a timeline showing events occurring during a rescue effort generated from the case file data including from the waveforms of FIGS. 10A and 10B;

FIG. 10D is a Transition Time Report showing transition times for ventilation events occurring during a rescue effort generated based on data from a case file, according to an aspect of the present disclosure;

FIG. 10E is a another example of a report showing transition times between types of ventilations during a rescue effort generated based on data from a case file, according to an aspect of the present disclosure;

FIG. 11A is a flow chart showing a method for generating a case file from ventilation data captured by airflow sensor(s) at a rescue scene, which can be performed by a medical device at the rescue scene or by a computing device at a rescue scene or remote from the rescue scene, according to an aspect of the disclosure;

FIG. 11B is a flow chart showing a method for generating a transition time report for events occurring during a rescue effort from ventilation case file data, according to an aspect of the present disclosure;

FIG. 12A is a schematic drawing of a system for monitoring and determining transition times between activities performed by multiple rescuers during a rescue event, according to an aspect of the present disclosure;

FIG. 12B is a schematic drawing of another exemplary system for monitoring and determining transition times between activities performed by multiple rescuers during a rescue event, according to an aspect of the present disclosure;

FIG. 12C is a schematic drawing showing rescuers using the system of FIG. 12A to provide care for a patient during a rescue effort, according to an aspect of the present disclosure;

FIGS. 13A-13C are schematic drawings of wrist-worn devices of the systems of FIGS. 12A and 12B, which can be worn by rescuers to track rescuer movements during a rescue effort, according to an aspect of the present disclosure;

FIG. 14A is an annotated timeline showing actions and events occurring during a rescue effort generated from case file data, according to an aspect of the present disclosure;

FIG. 14B is a Patient Care Summary Report including transition time information for actions and events performed by different rescuers during a rescue effort, according to an aspect of the present disclosure;

FIG. 15A is a schematic drawing of a system for monitoring and determining transition times between heart attack events and post-heart attack user inputs occurring during a rescue effort, according to an aspect of the present disclosure;

FIG. 15B is a flow chart showing a method for generating a transition time report for transition times between heart attack events and post-heart attack user inputs, according to an aspect of the present disclosure;

FIG. 16 is a Monthly CPR Trends Report including average transition time information for rescue efforts occurring during different months, according to an aspect of the present disclosure;

FIG. 17A is a schematic drawing of a system for providing feedback about resuscitation activities performed for a patient at a rescue scene including transition time information and associated alarms and notifications, according to an aspect of the present disclosure;

FIG. 17B is a flow chart showing a method for providing transition time feedback for resuscitation activities performed for a patient during a rescue effort, according to an aspect of the disclosure; and

FIGS. 17C and 17D are drawings of defibrillator display screens showing resuscitation activity feedback that can be provided to rescuers during a rescue effort, according to aspects of the present disclosure.

### DETAILED DESCRIPTION

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the disclosure.

As used herein, the singular form of “a”, “an”, and “the” include plural referents unless the context clearly dictates otherwise.

As used herein, the terms “right”, “left”, “top”, and derivatives thereof shall relate to aspects of the present disclosure as it is oriented in the drawing figures. However, it is to be understood that embodiments of the present disclosure can assume various alternative orientations and, accordingly, such terms are not to be considered as limiting. Also, it is to be understood that embodiments of the present disclosure can assume various alternative variations and stage sequences, except where expressly specified to the contrary. It is also to be understood that the specific devices and processes illustrated in the attached drawings, and described in the following specification, are provided as examples. Hence, specific dimensions and other physical characteristics related to the embodiments disclosed herein are not to be considered as limiting.

As used herein, including in the claims, “and” as used in a list of items prefaced by “at least one of” indicates a disjunctive list such that, for example, a list of “at least one of A, B, and C” means A or B or C or AB or AC or BC or ABC (i.e., A and B and C), or combinations with more than one feature (e.g., AA, AAB, ABBC, etc.). As used herein, including in the claims, unless otherwise stated, a statement that a function or operation is “based on” an item or condition means that the function or operation is based on the stated item or condition and may be based on one or more items and/or conditions in addition to the stated item or condition.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that can be wired and/or wireless in nature. Additionally, two units or components can be in communication with each other even though the data transmitted can be modified, processed, routed, and the like, between the first and second unit or component. For example, a first unit can be in communication with a second unit even though the first unit passively receives data, and does not actively transmit data to the second unit. As another example, a first unit can be in communication with a second unit if an intermediary unit processes data from one unit and transmits processed data to the second unit. It will be appreciated that numerous other arrangements are possible.

Systems and methods for assisting rescuers, such as acute care providers, to treat patients during medical emergencies, particularly cardiac arrest, are disclosed herein. Medical emergencies require a rapid response to increase the likelihood of achieving a positive outcome for the patient and to provide the best chance for patient survival. In particular, it is important that care is provided quickly to patients and that, once care for the patient begins, any delays or pauses in ongoing patient care are minimized or eliminated.

In some examples, the systems and methods of the present disclosure are provided to monitor, track, and report transition times between different aspects of patient care during a rescue effort. Measured transition time information can be provided to rescuers and other users following a rescue event so that measured transition time information can be considered during, for example, a code review or debrief session. The systems and methods of the present disclosure can track transition times between events occurring during a rescue effort and, in particular, between changes in types of patient care to provide quantitative information about delays or pauses in patient care during the rescue effort. For example, the systems and methods of the present disclosure can track a transition time between manual and automated chest compression, between manual and automated ventilations, and/or a transition time that occurs when rescuers switch roles during a rescue effort. The systems and methods of the present disclosure may also track how long it takes to set up a medical device at a rescue scene and/or a time between arrival at the scene and a start of chest compressions or ventilations. This transition time information can be used by teams of rescuers to improve efficiency and, in particular, to identify areas of the rescue effort where transition times can be reduced to improve continuity of patient care.

### Chest Compression Transition Time Reporting Systems

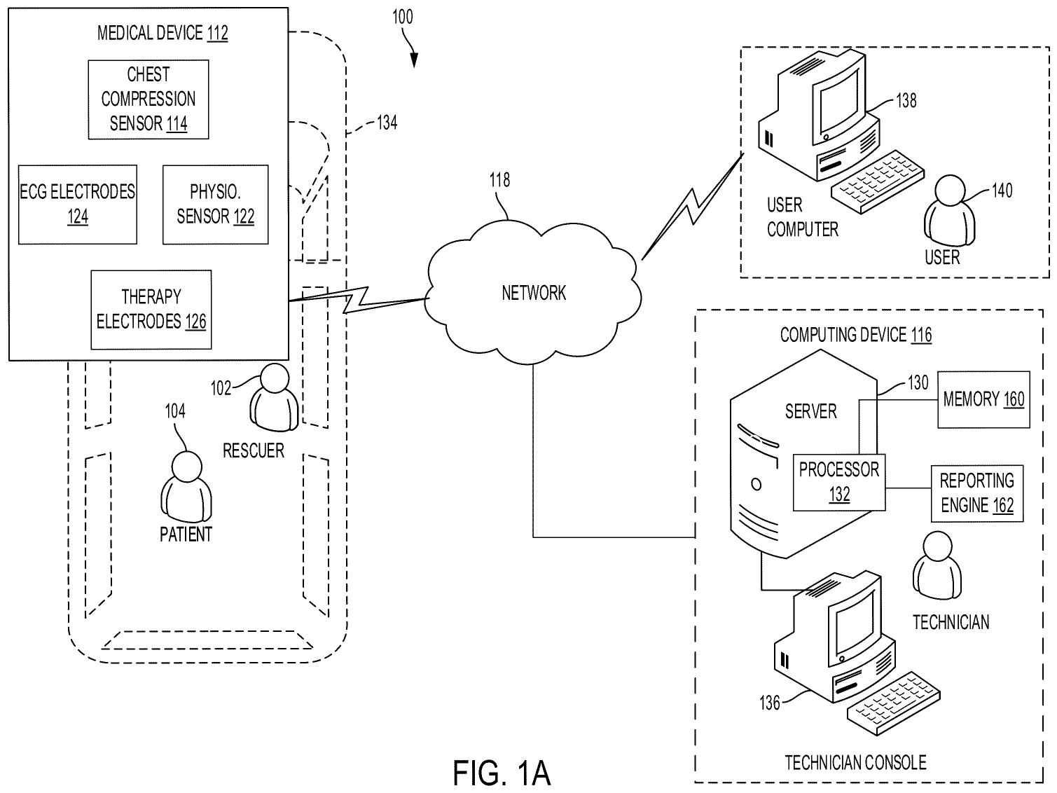

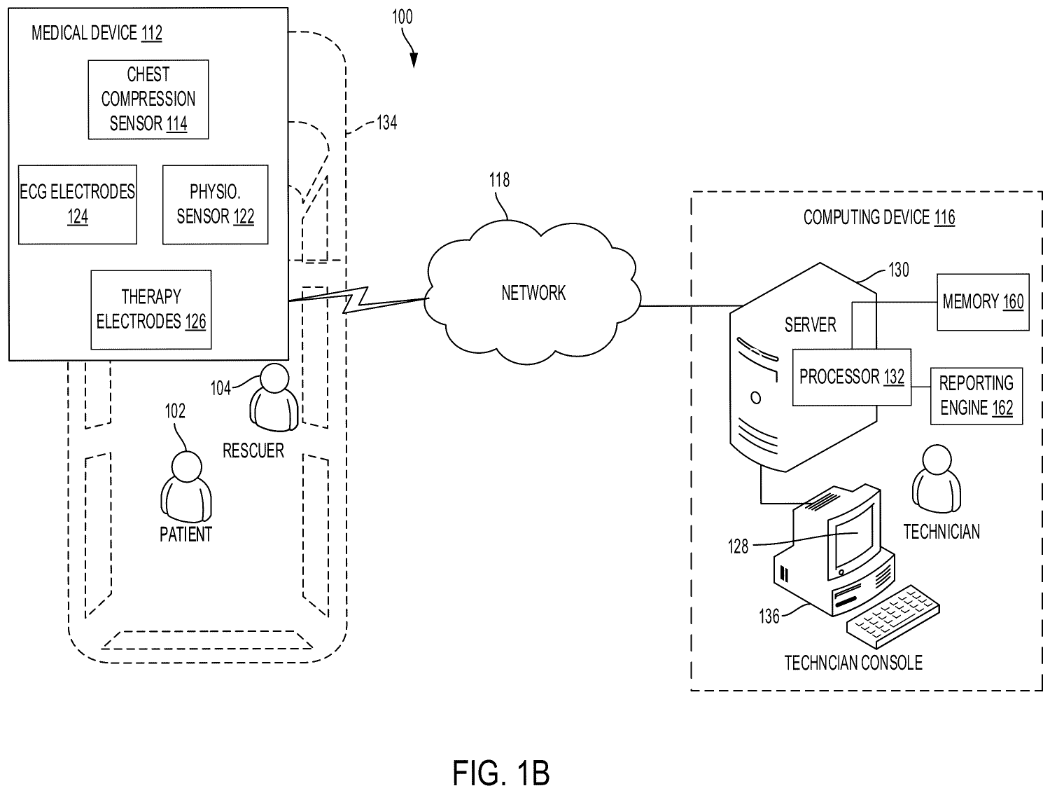

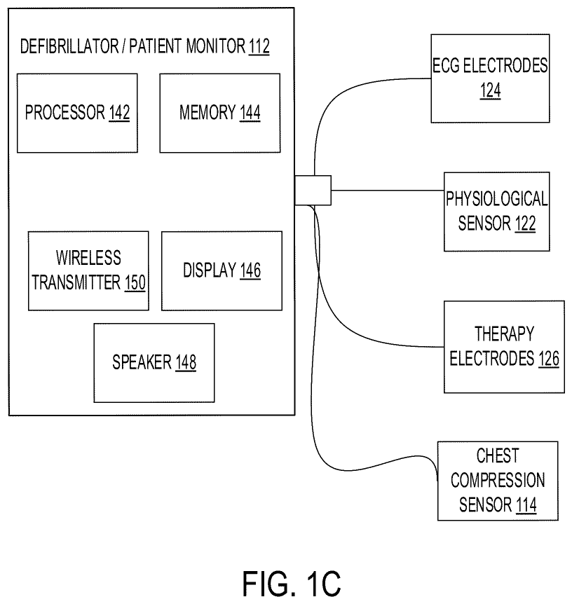

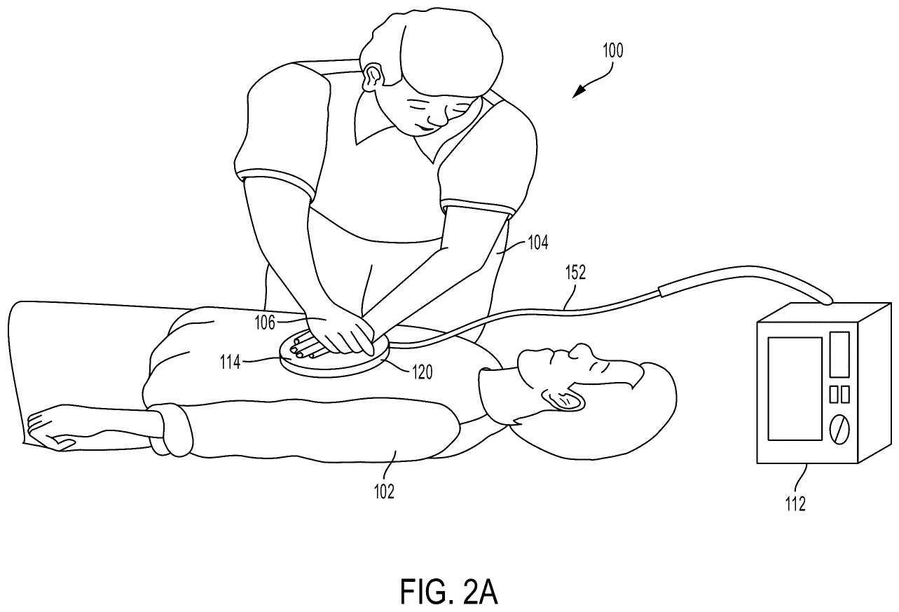

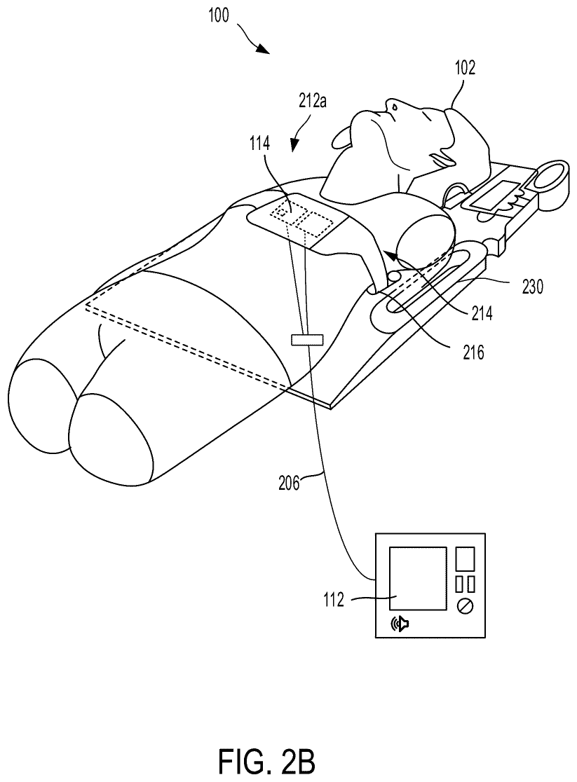

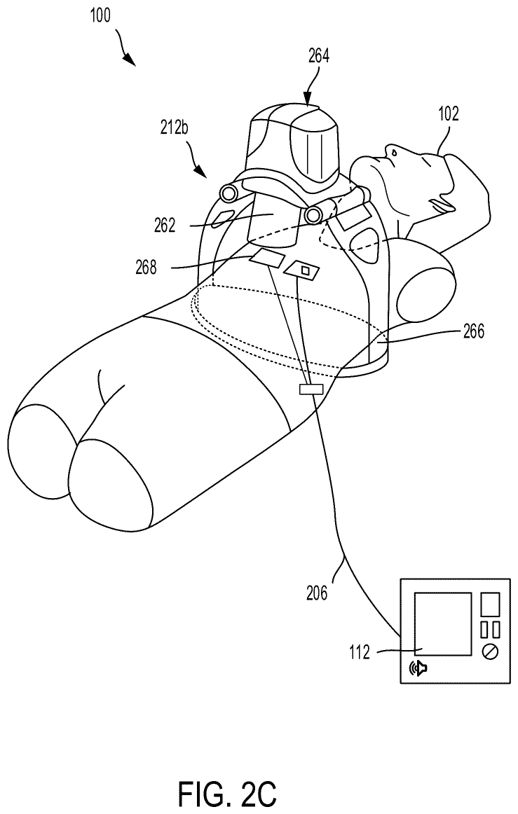

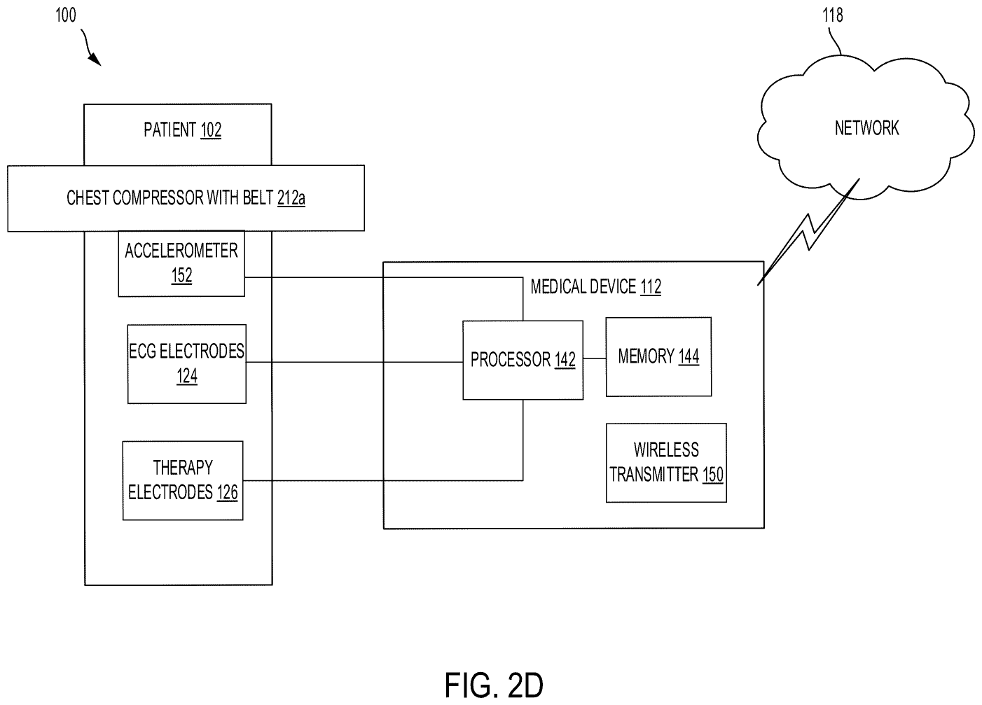

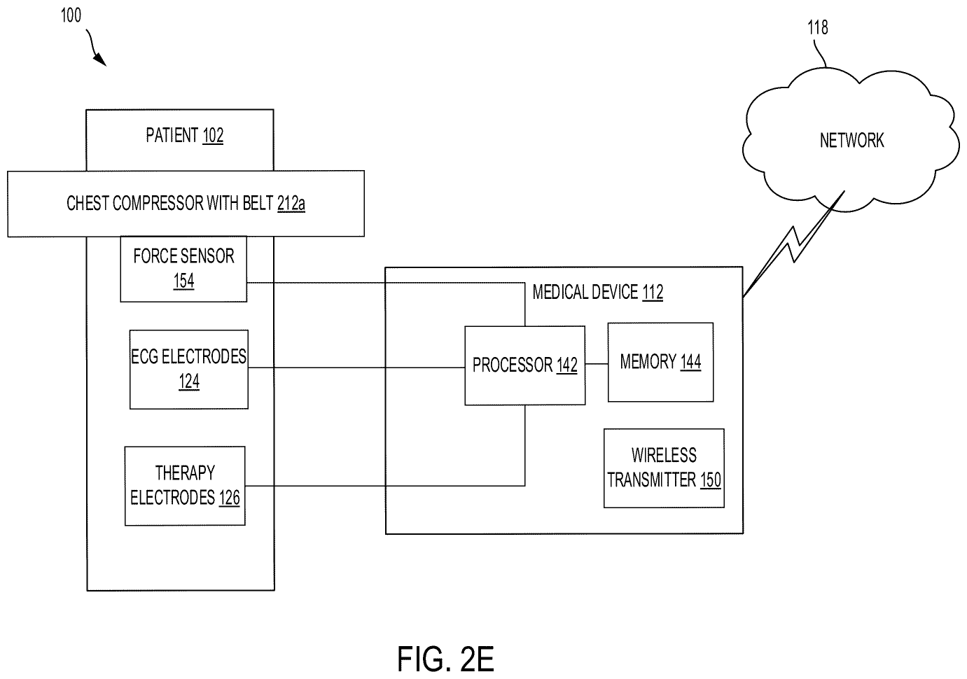

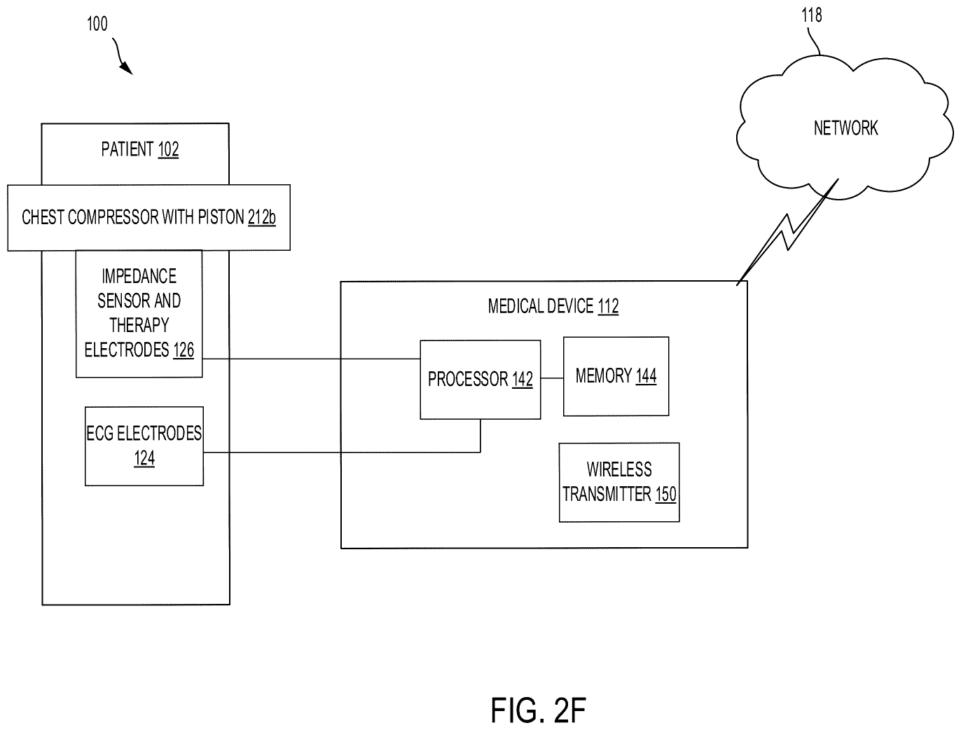

FIGS. 1A-2C illustrate devices and components of systems 100 for monitoring and/or reviewing transitions between types of chest compressions provided for a patient 102 by a rescuer 104. The systems 100 comprise a medical device 112, such as defibrillator and/or patient monitor, comprising a chest compression sensor 114 configured to receive time-correlated signals representative of chest compressions performed for the patient 102 and a computing device 116 communicatively coupled to the medical device 112. As described in further detail herein, in some examples, the computing device 116 is a local portable computing device present at a rescue scene and in wired or wireless communication with the medical device 112. For example, the local portable computing device can be a personal computer, smart phone, computer tablet, or similar electronic device. The computing device 116 can also be integral with and/or a component of the medical device 112 or of another medical device (e.g., a defibrillator, automated external defibrillator, ventilator, automated chest compression device, patient monitor, etc.) at the rescue scene. For example, one or more computer processors of the medical device 112 can be configured to perform the monitoring and transition time review processes performed by the computing device 116 and reports generated from the review can be displayed to a user on a display or another feedback component of the medical device 112. In other examples, the computing device 116 can be a remote computing device (e.g., a remote computer, a computer network, a central server, a hospital computer server, etc.) that is not present at the rescue scene. In such cases, the time correlated signals can be transmitted from the medical device 112 to the remote computing device 116, which analyzes the received signals in order to monitor and review the transition times. The remote computing device 116 can also generate a report that provides the transition times to a user. For example, the generated report can be provided to a user, such as a clinician, quality review user, and/or service technician, on a display of a relevant computing portal or device.

In some examples, the communicative coupling between the medical device 112 and the computing device 116 may be a remote connection via a cloud based internet network. Though, in some embodiments, this communicative coupling between the medical device 112 and the computing device 116 may be a local connection, such as via BLUETOOTH, a direct cable connection, or another local communications protocol. The computing device 116 is configured to generate a report for user review that provides a transition time indication representative of a transition time between two or more events occurring during a rescue effort.

As described herein, a transition time may refer to an elapsed time, pause, or delay between a first event of a rescue effort and a second event of the rescuer effort. In most cases, the transition time between events of patient care should be as short as possible to ensure continuity of care between interventions and/or pauses and, for chest compressions, to ensure that blood flow through patient vasculature is not interrupted. For example, a transition time between an end of one type of compressions (e.g., manual chest compressions) and another type of chest compressions (e.g., automated chest compression) desirably should be limited to less than 5 minutes, 2 minutes, 60 seconds, 30 seconds, 15 seconds, or 10 seconds. The transition times reported by the system 100 can be, for example, a time between at least one of: (i) turning on the at least one medical device and a start of manual chest compressions, (ii) turning on the at least one medical device and an end of manual chest compressions, (iii) turning on the at least medical device and a start of automated chest compressions, (iv) turning on the at least one medical device and an end of automated chest compressions, (v) the start of the manual chest compressions and the end of the manual chest compressions, (vi) the start of the manual chest compressions and the start of the automated chest compressions, (vii) the start of the manual chest compressions and the end of the automated chest compressions, (viii) the end of the manual chest compressions and the start of the automated chest compressions, (ix) the end of the manual chest compressions and the end of the automated chest compressions, or (x) the start of the automated chest compressions and the end of the automated chest compressions.

The systems 100 and devices of the present disclosure can be configured to process, evaluate, and/or analyze the time-correlated signals from the chest compression sensor 114 to calculate or determine the transition times and to generate the reports for user review. In some examples, processing of the compression signals can be primarily performed on a controller or processor of the medical device, such as the medical device 112. For example, the medical device 112 can be configured to receive and process the time-correlated signals from the chest compression sensor 114 and identify and determine a time of occurrence for a plurality of events represented in the time-correlated signals. In other examples, some or all of the processing of the time-correlated compression signals can be performed by the computing device 116. For example, the medical device 112 can be configured to generate or prepare a case file comprising the signals received from the chest compression sensor 114. In that case, for certain embodiments, a substantial portion or majority of signal processing including identification of events and/or determination of times of occurrence for the events can be performed by the processor of the computing device 116. Accordingly, advantages provided by embodiments of the present disclosure allow for efficient review of significant data that would otherwise be too onerous for a user to manage in an organized and effective manner. Otherwise, a user would have to manually sift through massive amounts of data, accurately mark notable events of interest, and then determine each of the particular transition times for further review.

More specifically, in some examples, as described in further detail herein, the medical device 112 and/or the computing device 116 can be configured to identify and determine the time of occurrence for an end of the manual chest compressions by: generating a compression waveform representation from the received and processed time-correlated signals; identifying portions of the compression waveform representative of manual chest compressions provided for the patient; and determining a final time of the portions of the at least one compression waveform representative of the manual chest compressions. More specifically, in some examples, the medical device 112 (e.g., the defibrillator or patient monitor) and/or the computing device 116 can be configured to identify the portions of the chest compression waveform representative of manual chest compressions by calculating a chest compression parameter value for multiple segments of the compression waveform. The chest compression parameter can be compression rate, compression depth, compression hold time, variation in compression rate, variation in compression depth, variation in hold time, compression width, relaxation time, release time, compression average velocity, compression maximum velocity, or velocity minimum to maximum time (per chest compression cycle). The medical device 112 can then be configured to compare the calculated chest compression parameter value for the multiple segments to a target range for the chest compression parameter values representative of manual chest compressions and to identify segments of the compression waveform as “manual chest compression segments” having a chest compression parameter value within the target range for manual chest compressions. In a similar manner, the medical device 112 can be configured to identify and determine the time of occurrence for the start of the automated chest compressions by: identifying portions of the compression waveform representative of automated chest compressions provided for the patient and determining a first time of the portions of the compression waveform representative of the automated chest compressions.

Once the events occurring during the rescue effort are identified, the medical device 112 and/or the computing device 116 can be configured to generate a case file for the rescue effort comprising the times of occurrence for the plurality of medical events represented in the time-correlated signals. In some examples, the case file can then be transmitted (e.g., uploaded) from the medical device 112 to the computing device 116 via a network 118. Alternatively, if the computing device 116 is local to the medical device 112, the case file can be transmitted locally over, for example, a short-range wireless transmitter (e.g., BLUETOOTH) or a direct cable connection.

Once the case file is generated, the medical device 112 and/or the computing device 116 can be configured to select and obtain or determine the time of occurrence for a first event of the plurality of medical events from the case file. The first event can include, for example, turning on the medical device, the start of manual compressions, the end of manual compressions, the start of automated chest compressions, or the end of automated chest compressions. The medical device 112 and/or the computing device 116 can also select and determine the time of occurrence for a second event of the plurality of medical events from the case file occurring after the selected first event. The second event can comprise, for example, the start of manual chest compressions, the end of manual chest compressions, the start of automated chest compressions, or the end of automated chest compressions. The medical device 112 and/or the computing device 116 can further be configured to determine the transition time between the time of occurrence of the first event and the time of occurrence of the second event, and generate the report that provides a transition time indication representative of the determined or calculated transition time for user review.

With continued reference to FIGS. 1A-2C, the medical device 112 of the present disclosure can be any medical device for monitoring and/or providing therapy to a patient, as are known in the art. The medical device 112 may be, for example, a patient monitor, a defibrillator, a mechanical chest compression device (e.g., an automated chest compression device, a belt-based chest compression device, a piston-based chest compression device, an active compression-decompression device, or combinations thereof), a ventilator, an intravenous cooling device, and/or combinations thereof. The ventilator may be a mechanical ventilator. The mechanical ventilator may be a portable, battery powered ventilator. The intravenous cooling device may deliver cooling therapy and/or may sense a patient's temperature. The medical device 112 may provide, for example, electrical therapy (e.g., defibrillation, cardiac pacing, synchronized cardioversion, diaphragmatic stimulation, and/or phrenic nerve stimulation), ventilation therapy, therapeutic cooling, temperature management therapy, invasive hemodynamic support therapy (e.g., extracorporeal membrane oxygenation (ECMO)), and/or combinations thereof. The medical device 112 may also be a wearable device (not shown), such as a smartwatch, worn by an acute care provider for providing alarms, notifications, and feedback about the chest compressions.

In some examples, the medical device 112 is a portable medical device, such as a portable patient monitor, portable defibrillator, portable ventilator, or another portable medical device, used for treating a patient during a rescue event remote from a hospital or medical facility. As previously described, medical device 112 can comprise or can be connected to the chest compression sensor 114, which is configured to receive time-correlated signals representative of chest compressions performed for the patient.

In some examples, the chest compression sensor 114 can be a single axis accelerometer or a multi-axis accelerometer, as are known in the art. Compression depth for chest compressions provided to the patient 102 may be estimated by double-integration of acceleration signals. In some examples, the chest compression sensor 114 can also comprise a velocity sensor or a displacement sensor other than an accelerometer for detecting features of compressions provided to the patient 102. In that case, compression depth can be determined by integration of the velocity sensor signal. In other examples, the chest compression sensor 114 can comprise a velocity sensor for measuring a velocity of the patient's chest during chest compressions. For example, the velocity sensor and an electromagnetic field generator positioned proximate to the patient (e.g., magnet and conductor positioned on opposite sides of the patient, one at an anterior location and the other at a posterior location during chest compressions). The sensor can be configured to measure velocity at which the magnet moves through the electromagnetic field as chest compressions are being performed in order to determine compression velocity and/or compression rate. Velocity measurements can also be integrated to determine displacement, which can be used to determine compression depth.

The chest compression sensor 114 can also be a thoracic impedance sensor, such as therapy electrodes 126 (shown in FIGS. 1A-1C) of a defibrillator for providing cardiac therapy for a patient. Impedance signals detected by the impedance sensor can be monitored to identify changes in thoracic impedance, which occur as the chest is compressed and released during chest compressions. In various embodiments, the therapy electrodes 126 include electrodes positioned on opposite sides of the patient's heart and are configured not only to provide electrotherapeutic treatment to a patient, but may also be used to measure thoracic impedance of the patient, as well as collect ECG signals.

In other examples, the chest compression sensor 114 can comprise a force sensor or force sensing system configured to sense information representative of force applied to the patient's chest during chest compressions. The force sensor can be a strain gauge configured to convert a force, pressure, tension, or weight, into a measurable electrical resistance. In other examples, the force sensor comprises a spring with a known spring constant. Alternatively, the force sensor can comprise a pressure sensor, which measures an amount of applied pressure.

Force measurements can be analyzed to estimate compression rate or depth (in conjunction with a measurement of displacement or compliance). For example, when using force to estimate depth, the compliance or stiffness of the chest will affect how the chest deforms. Accordingly, when the compliance of the chest is known or can be estimated with reasonable accuracy, the depth can also be estimated from a force measurement. Also, force measurements can be used as input for a chest compression system (e.g., used for manual compressions and/or automated compressions) to adjust a target depth of remaining chest compressions of an initial compression protocol. Information and signals detected and output by the compression sensor 114 may be represented using any of a variety of different technologies and techniques. For example, information or signals detected or output by the sensor(s) may be represented by voltages, currents, electromagnetic waves, magnetic fields, or any combination thereof, which may be processed in a manner that is useable to estimate physical measurements, such as force, displacement, compliance, etc.

An exemplary chest compression sensor 114, such as the accelerometer, positioned on the patient's sternum is shown in FIG. 2A. The compression sensor 114 is enclosed in a housing 120, which is positioned on the patient 102 between hands 106 of the rescuer 104 performing the chest compressions and the patient's sternum. To perform the chest compressions, the rescuer 104 presses on and releases the housing 120 of the chest compression sensor 114. The chest compression sensor 114 can be electrically connected to other components of the medical device 112 by a wire 152 or cable extending from the compression sensor 114 to the medical device 112 for providing the chest compression signals to the medical device 112 for processing and analysis, so that the medical device 112 is able to track relevant compression parameters (e.g., compression depth, compression rate, release velocity) of the compressions provided by the rescuer 104 to determine whether those compression parameters are within desired target ranges.

With continued reference to FIGS. 1A-2C, the medical device 112 can be a patient monitor that receives and processes signals from the various sensors. The patient monitor can comprise a display for displaying indications and numerical values for vital signs detected by the sensors. The patient monitor can also be configured to emit alarms or notifications generated based on analysis of signals detected by the sensors. In some examples, the medical device 112 or patient monitor can comprise a patient physiological sensor 122 configured to detect signals representative of patient vital signs. Patient vital signs detected by the medical device 112 and/or physiological sensor 122 can comprise, for example, blood pressure (e.g., invasive blood pressure (IBP), non-invasive blood pressure (NIBP)), heart rate, pulse oxygen level, respiration rate, heart sounds, lung sounds, respiration sounds, end tidal CO₂, saturation of muscle oxygen (SMO₂), arterial oxygen saturation (SpO₂), cerebral blood flow, electroencephalogram (EEG) signals, brain oxygen level, tissue pH, tissue oxygenation, or tissue fluid levels.

In some examples, medical device 112 can further comprise an electrocardiogram (ECG) sensor or ECG electrodes 124 for detecting ECG signals of the patient 102. In that case, a controller, such as a computer processor of the medical device 112, can be configured to monitor signals detected by the ECG electrodes 124 to collect data to identify outcome information for a rescue effort, such as whether a return to spontaneous circulation (ROSC) occurred during the rescue effort and/or whether cardiac arrest events or heart attack events occurred during the rescue effort. The controller of the medical device 112, such as the computer processor, can also be configured to cause information about the ROSC, the cardiac arrest event, or the heart attack event to be displayed on a visual display of the medical device 112 along with patient vital sign information detected by the physiological sensors 122. In some instances, the outcome information can be displayed on the medical device 112 along with and/or in proximity to the transition time indication.

In some examples, the medical device 112 is a defibrillator, such as a portable basic life support (BLS) and/or advanced life support (ALS) defibrillator, or a public access automated external defibrillator (AED), such as the AED PLUS, or AED PRO from ZOLL Medical Corporation of Chelmsford, Mass. The defibrillator may also be a professional style defibrillator, such as the X SERIES, R SERIES, M SERIES, or E SERIES provided by ZOLL Medical Corporation. As shown in FIGS. 1A-1C, the defibrillator can comprise therapy electrodes 126 for providing cardiac therapy for the patient 102 based on an analysis of the signals detected by the ECG electrodes 124.

As previously described, the system 100 further comprises the computing device 116, which can comprise a reporting engine 162 comprising a processor 132 and computer readable memory 160. The computing device 116 can be a component of a medical device 112, a local portable computing device, or a remote computing device comprising the processor 142 and computer readable memory 160 of the reporting engine 162. The reporting engine 162 can be configured to generate the report comprising the transition time indication from the case file prepared by the medical device 112. The reporting engine 162 can comprise hardware logic on the processor 132 and/or software logic stored on the memory 160 configured to receive the case file and generate the report and other metrics for a rescue event and provides a graphical user interface (GUI) that displays the report. In particular, the memory 160 and/or the reporting engine 162 can comprise processor-executable instructions configured to cause the processor 132 to receive the case file and generate the report and other metrics for a rescue event. The instructions can also cause the processor 132 to provide the GUI that displays the report on a display 128 of the computing device 116 communicatively coupled with the reporting engine 162 and processor 132.

In some examples, the reporting engine 162 and/or processor 132 of the computing device 116 can be configured to receive the case file for the rescue effort from the medical device 112 or from another computing device at the rescue scene via the network 118. As previously described, the reporting engine 162 and/or the computing device 116 is further configured to select and determine a time of occurrence for a first event of the plurality of medical events from the case file, select and determine a time of occurrence for a second event of the plurality of medical events from the case file occurring after the selected first event, determine a transition time between the time of occurrence of the first event and the time of occurrence of the second event, and generate a report that provides a transition time indication representative of the determined or calculated transition time for user review. In some examples, the transition time indication is provided on the display 128 in real-time during a rescue effort. In other examples, reported transition times can be reviewed after completion of a rescue effort as part of a debrief following the rescue effort or during a post-case quality review, so as to evaluate medical personnel performance and improve training/education thereof. Alternatively, the medical device 112 or another computing device 116 at a rescue scene can be configured to determine the transition time and/or display the transition time indication from a case file generated by the medical device 112.

As noted herein, the computing device 116 and/or reporting engine 162 can be located at the rescue scene or remote from the rescue scene. For example, the computing device 116 and/or reporting engine 162 can be a component or extension of the medical device 112, such as a computer processor of the medical device 112, which also controls operation of the medical device 112. In other examples, the computing device 116 and/or reporting engine 162 can be a separate processor or device directly connected to or associated with the medical device 112. For example, the computing device 116 and/or reporting engine 162 can be a separate computer processor enclosed within a housing of the medical device 112, but which is separate from the processor and/or circuitry that controls the medical device 112. In still other examples, the computing device 116 and/or reporting engine 162 can be a portable computing device configured for use at a rescue scene during the rescue effort. For example, the computing device 116 can be a computer tablet, smartphone, or similar electronic device that provides a rescuer with information about a rescue effort in real time over the course of the rescue effort.

In other examples, the processor 132, memory 160, and/or reporting engine 162 of the computing device 116 can be components of a remote computer server 130 or technician terminal that receives information about the rescue effort, such as the case file, and generates the report. In that case, the server 130 can also be configured to make the report available for user review over a computer network 118 (e.g., a local network, limited access network, or the Internet) for review by users.

FIGS. 1A-1C show exemplary systems 100 that are configured to generate and provide the report comprising the transition time indication for user review. For example, FIG. 1A illustrates an example of a system 100 comprising the medical device 112, such as a patient monitor or defibrillator, in communication with the computing device 116 via a computer network 118. The medical device 100 comprises the chest compression sensor 114, the ECG electrode(s) 124, the physiological sensor 122, and the therapy electrodes 126 for providing cardiac therapy to the patient 102. The medical device 112 is positioned in and/or being transported to a rescue effort by an emergency response vehicle, such as an ambulance 134. The computing device 116 comprises the server 130, which comprises the processor 132, memory 160, and reporting engine 162 for generating the report from the case file. The computing device 116 can also comprise a technician terminal 136, which can be used by a technician to review the report. The system 100 further comprises a user computer 138 that is in communication with the server 130 via the computer network 118. A user 140 can review the report on a website on the user computer 138.

FIG. 1B shows another embodiment of a system 100 comprising the medical device 112, in the form of a patient monitor or defibrillator, connected to the computing device 116 via the computer network 118. As shown in FIG. 1B, the computing device 116 comprises the computer server 130 comprising the processor 132, memory 160, and reporting engine 162 for receiving the case file and generating the report. The computing device 116 also comprises a technician terminal 136 comprising the display 128. The report can be displayed on the technician terminal 136 for review by a technician.

FIG. 1C is a schematic drawing of another embodiment of a medical device 112, such as a patient monitor or defibrillator, including electrical components and circuitry for generating and displaying the report to a user 140 directly on the medical device 112. That is, for some embodiments, the medical device 112 may be configured to process the time-correlated signals from the chest compression sensor 114 and generate not only the case file, but also the report that includes relevant transition times between notable events. As in previous examples, the medical device 112 of FIG. 1C comprises the compression sensor 114 for detecting chest compressions performed for the patient 102 (shown in FIGS. 1A and 1B). The medical device 112 also comprises sensors for detecting patient condition, such as the physiological sensor 122 and the ECG electrodes 124. The medical device 112 also comprises the therapy electrodes 126 for providing cardiac therapy for the patient 102.

The medical device 112 of the embodiment illustrated in FIG. 1C further comprises a processor 142 and memory 144 for generating the report comprising the transition time indication. In particular, the processor 142 and memory 144 can be configured to cause the medical device 112 to receive and process the compression signals from the compression sensor 114 to generate the case file. The processor 142 and memory 144 can also be configured to evaluate the case file to determine a transition time between a first event and a second event from the case file, as previously described. The medical device 112 can also comprise output components, such as a visual display 146 and speaker 148 for providing the generated report, as well as other information about the patient 102 or rescue effort for review by rescuers 104 at the rescue scene. The medical device 112 can also comprise a wireless transmitter 150 transmitting the case file and/or generated transition time report to a remote device or computer network, such as any of the remote computing devices shown in FIGS. 1A and 1B.

With reference to FIGS. 2B and 2C, the system 100 can further comprise an automated or mechanical chest compressor 212a, 212b configured to be positioned on a chest of the patient 102 for providing the automated chest compressions for the patient 102. Mechanical or automated chest compressors, which can be used with the systems 100 and methods of the present disclosure are available from numerous manufacturers including ZOLL Medical Corporation, and others that provide similar or same types of therapy. Automated chest compressors 212a, 212b generally comprise a compression surface, such as a belt 214 or pad 268, configured to be positioned on the patient's chest. The automated chest compressor 212a, 212b further comprises a driver configured to move the compression surface in a first direction to compress the patient's chest and in a second direction to release the patient's chest. As described in further detail herein, the driver can be, for example, a motor, such as a belt-tensioner, for rotating a spindle to wind the belt 214 onto the spindle, thereby applying a chest compression. The motor can also cause the spindle to rotate in an opposite direction to release the belt 214 and the chest compression. The motor can also comprise a linear actuator configured to drive a piston 262 (shown in FIG. 2C) against the patient's chest to perform chest compressions.

In some examples, as shown in FIG. 2B, the chest compressor 212a comprises the compression belt 214 and a belt tensioner configured to tighten the compression belt 214 around the chest of the patient 102 in order to compress the patient's chest. One example of a belt-based chest compressor 212a is the ZOLL® AutoPulse®. In some examples, a displacement sensor, such as a chest compression sensor 114, can be mounted to the compression belt 214, as shown in FIG. 2B. Other sensors, such as a force sensor or a physiological sensors 122, may also be coupled to the compression belt 214. In some examples, the sensors 114, 122 may be a component of a defibrillation electrode assembly and/or used in conjunction and/or coordination with a defibrillation electrode assembly. The sensors 114, 122 may send signals indicative of the motion of the patient's chest to the medical device 112 via a wired and/or wireless connection, such as by the wire 206.

In other examples, as shown in FIG. 2C, the chest compressor 212b of the system 100 can be a piston-based device comprising a piston 262, a piston driver 264, support structures 266 for supporting the piston 262 and the piston driver 264, and the compression pad 268 configured to be affixed to the piston 262 or to the patient 102 during chest compressions. The piston driver 264 can comprise and/or can be coupled to a linear actuator motor for moving the piston 262 downward to provide the chest compressions for the patient 102 and upwards away from the patient's chest to release the compression. The compressor 212b further comprises the support structures 266 for supporting the piston 262 and the piston driver 264. The support structure 266 can be mounted to a backboard (not shown) for maintaining proper positioning of the support structure 266 relative to the patient 102, ensuring that the piston 262 is properly placed to provide effective chest compressions for the patient 102. As in previous examples, the chest compressor 212b can comprise sensors, such as the chest compression sensor 114 and/or the physiological sensors 122, for monitoring the chest compressions and patient condition, as previously described.

FIGS. 2D-2F show examples of systems 100 comprising the mechanical or automated chest compressors, such as the chest compressor 212a with a belt (shown in FIG. 2B) or the piston chest compressor 212b (shown in FIG. 2C), for providing chest compressions to the patient 102. The system 100 may further comprise one or more different types of sensors, such as examples of the previously described chest compressor sensors 114 for detecting chest compressions, as well as physiological sensors 122 and/or ECG electrodes 124 for detecting patient condition information.

The system 100 further comprises the medical device 112, which receives information from the different sensors and generates the case file. As in previous examples, the medical device 112 comprises a processor 142 and computer readable memory 144, which can comprise instructions that when executed by the processor 142 cause the processor 142 to receive and process sensor data to identify events occurring during the rescue effort and to generate the case file for the identified events. The medical device 112 can further comprise a wireless transmitter 150 that transmits the generated case file from the medical device 112 to remote computing devices, such as the computing device 116 shown in FIGS. 1A and 1B, via a cloud network 118.

More specifically, as shown in FIG. 2D, the system 100 can comprise a chest compressor 212a with a belt positioned over a chest of the patient 102. The system 100 further comprises a chest compression sensor, specifically an accelerometer 152, as well as ECG electrodes 124 and therapy electrodes 126 for providing cardiac therapy for the patient 102. As previously described, the chest compression sensor, e.g., the accelerometer 152, is positioned on the patient's chest and able to generate signals to detect manual chest compressions and automated chest compressions provided to the patient 102. Signals from the accelerometer 152 are provided to the processor 142 of the medical device 112 and are processed to distinguish between portions of the signal(s) from manual chest compressions and portions of the signal(s) from automated chest compressions, specifically automated chest compressions provided by a chest compressor 212a with a belt, as shown in FIG. 2D. In particular, as described in further detail herein, the medical device 112 can analyze accelerometer signals to distinguish between the different types of compressions based, for example, on whether parameter values derived from the accelerometer signals are within a target range of values or otherwise meet specified criteria for manual chest compressions, or are within a target range of values or otherwise meet specified criteria for automated chest compressions. In some examples, the medical device 112 can also be configured to analyze accelerometer signals to distinguish between automated chest compressions provided by the chest compressor 212a with a belt and compressions by other types of mechanical chest compressors, such as the piston-based chest compressor 212b, as further noted herein.

With reference to FIG. 2E, another exemplary system 100 comprises a chest compressor 212a with a belt positioned over a chest of the patient 102 and a force sensor 154 positioned on the patient's chest to detect manual or automated chest compressions provided to the patient 102. The system 100 can also comprise the ECG electrodes 124 and therapy electrodes 126 for providing cardiac therapy for the patient 102. Signals from the force sensor 154 are provided to the processor 142 of the medical device 112 and are processed to distinguish between portions of the signal(s) from manual compression and portions of the signal(s) from automated chest compressions by the chest compressor 212a with the belt.

For example, the medical device 112 can analyze force signals provided by the force sensor 154 to distinguish between the different types of compressions. For example, automated chest compressions may have consistent force values, with a similar or nearly identical force applied to the patient's chest for each compression of a series of chest compressions. Force measurements obtained during manual chest compressions may have a greater variability in maximum force applied to the chest during a series of chest compressions. Chest compression rate may also be determined from force signals detected by the force sensor 154 and used to distinguish between manual and automated chest compressions. For example, compression rate for a series of automated chest compressions may be more consistent than compression rate for a series of manual chest compressions.

FIG. 2F shows another exemplary system 100 for monitoring chest compressions comprising a piston-based chest compressor 212b (as shown in FIG. 2C) applied over a chest of the patient 102. The system 100 also comprises an impedance sensor and therapy electrodes 126 for providing cardiac therapy for the patient 102 and for detecting thoracic impedance. For example, as previously described, the therapy electrodes 126 can be connected to a defibrillator to collect ECG signals and, if appropriate, further provide a shock for the patient 102. Additionally, the therapy electrodes 126 may also serve as an impedance sensor that generates signals indicative of thoracic impedance of the patient. The system 100 can further comprise ECG electrodes 124 for detecting an ECG signal of the patient 102 (e.g., 3-lead ECG, 12-lead ECG), as well as other physiological sensors 122 (not shown in FIGS. 2D-2F).

Signals from the impedance sensor and therapy electrodes 126 can be used to detect chest compressions. In particular, thoracic impedance can decrease as a compression is performed because air is forced from the lungs and because the anterior posterior (AP) distance from the chest to the back of the patient 102 decreases as the chest deforms when the compression is performed. The impedance increases when the compression is released allowing air to reenter the lungs and the AP distance to increase. Accordingly, the impedance signal detected by the electrodes 126 can be monitored by the processor 142 of the medical device 112 to detect a start and release of manual or automated chest compressions. Further, the impedance signal(s) from the electrodes 126 can be processed to distinguish between portions of the signal(s) for manual compression and portions of the signal(s) for automated chest compressions, such as the automated chest compressions provided by the chest compressor 212b with the piston, as shown in FIG. 2F. In particular, the medical device 112 can analyze the impedance signals for the manual and automated chest compressions to evaluate variability in chest compression parameters, such as compression rate or force/pressure. As previously described, a series of compressions having more uniform parameter values (e.g., compression rate, impedance amplitude) may be interpreted as originating from automated compressions performed by a mechanical chest compressor, while a series of compression with relatively greater variability (e.g., compression rate, impedance amplitude) in chest compression parameter values may be interpreted as originating from manual chest compressions.

### Analysis of Compression Data to Detect Events and Generate Reports

As previously described, for various embodiments, the medical device 112 and/or the computing device 116 of the present disclosure are configured to identify events occurring during the rescue effort by processing and analyzing chest compression signals provided by the chest compression sensor 114 of the medical device 112. In particular, the medical device 112 and/or portable computing device 116 can be configured to identify when the medical device 112 is turned on or activated and to distinguish between manual chest compressions (as shown in FIG. 2A) and automated chest compressions provided by the chest compressor 212a, 212b shown in FIGS. 2B and 2C. The medical device 112 and/or computing device 116 can also be configured to detect and/or record a time or event marker identifying a beginning or an end of manual and/or automated chest compressions.

As used herein, turning on of the medical device 112 and/or chest compression sensor 114 can refer to activation of the medical device 112 and/or chest compression sensor 114 to begin monitoring patient condition and/or resuscitation activities performed for the patient 102. For example, during a rescue effort, a rescuer 104 may arrive at a rescue scene, which can be identified as a case start time, and retrieve a portable medical device, such as a portable patient monitor or portable defibrillator, from an emergency response vehicle, such as an ambulance 134. The rescuer 104 may move the medical device 112 to a location in proximity to the patient 102 and, once the medical device 112 is in place, may attach sensors 114, 122 and/or electrodes 124, 126 of the medical device 112 to the patient 102. Once the sensors 114, 122 and/or electrodes 124, 126 are correctly connected to the patient 102, the rescuer 104 may activate the medical device 112 so that signals detected by the sensors 114, 122 and/or electrodes 124, 126 can be obtained and recorded in memory of the medical device 112. For purposes of analysis, the turn on or activation time for the medical device 112 can be an earliest or first time for which sensor data (e.g., signals obtained by the sensors 114, 122 or electrodes 124, 126) is available for a particular rescue effort.

The start of manual chest compressions may refer to an earliest time during the rescue effort that manual chest compressions are detected in the chest compression signal(s) detected by the chest compression sensor 114. Alternatively, a start time can refer to a time when manual compressions are restarted following a lengthy delay in providing manual chest compressions to the patient. The end of manual chest compressions may refer to a final time that manual chest compressions are detected in the chest compression signal and/or a last time that manual chest compressions are detected prior to a delay in providing manual chest compressions for the patient. The end of manual chest compressions may also refer to a final time that manual chest compressions are detected in the chest compression signal before transition to automated chest compressions using a mechanical compression device, such as the belt-based or piston-based devices described herein. It is noted, however, that during manual chest compressions, there will be short pauses or delays so that other types of treatment can be provided to the patient. For example, chest compressions can be delivered as chest compression cycles separated by brief ventilation cycles according, for example, to a predetermined CPR protocol, such as a protocol of 30 compressions followed by 2 ventilations (often referred to as a 30:2 CPR protocol). The time between compression cycles is not considered to be a “start” or “end” of manual chest compressions for purposes of determining the transition times in the reports generated by the system 100 of the present disclosure.

In a similar manner, the start of automated chest compressions can refer to an earliest time during the rescue effort that automated chest compressions are detected in the chest compression signal(s) from the chest compression sensor 114. Alternatively, a start time can refer to a time when automated chest compressions are restarted following a lengthy delay in providing automated chest compressions to the patient 102. The end of automated chest compressions may refer to a final time that automated chest compressions are detected in the chest compression signal and/or a last time that automated chest compressions are detected prior to a delay in providing automated chest compressions for the patient 102.

As previously described, the transition times can be determined by the medical device 112 and/or by the computing device 116 by processing and analyzing signals detected by the chest compression sensor 114. The processing and analysis of the chest compression signals can include distinguishing between types of chest compressions in the compression signals. In particular, the devices 112, 116 may determine which chest compressions in the recorded signals are manual chest compressions and which chest compressions are automated chest compressions. Distinguishing between manual chest compressions and automated chest compressions in signals detected by a chest compression sensor 114, such as an accelerometer, can be performed by analyzing data for displacement, velocity, and acceleration to identify portions of detected signals representative of manual chest compressions and portions of the detected signals representative of automated chest compressions.

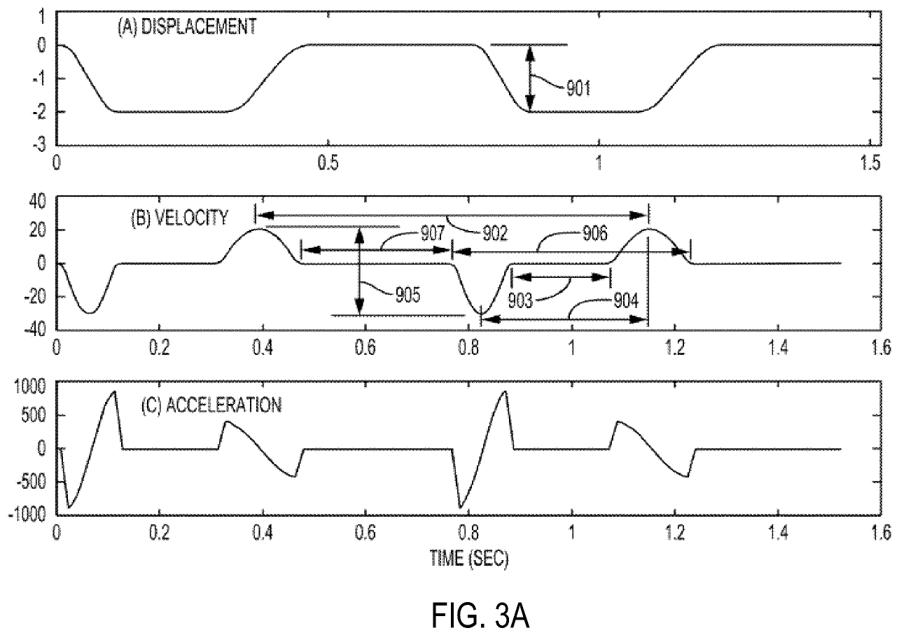

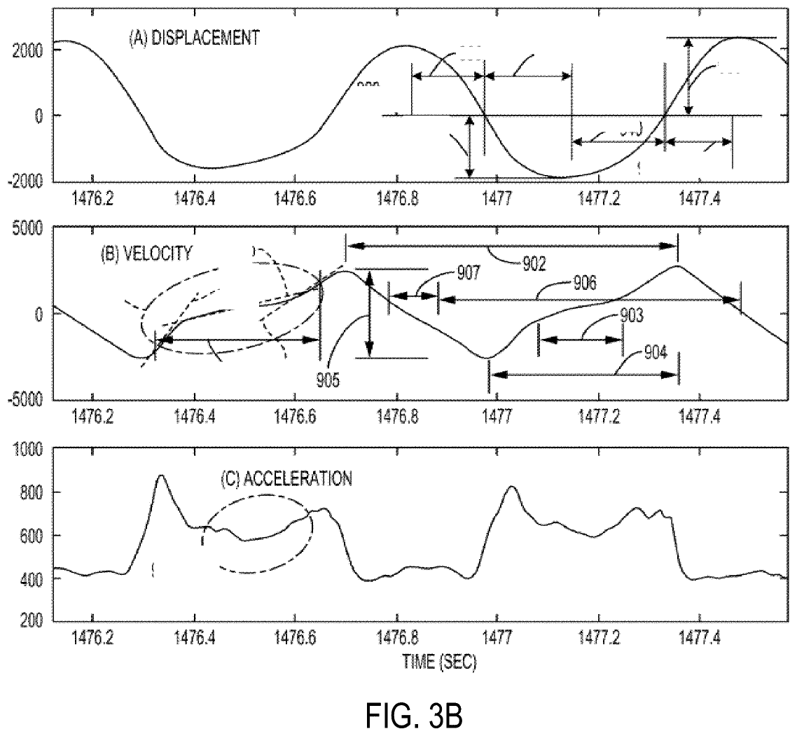

Exemplary waveforms for displacement, velocity, and acceleration for chest compressions are shown in FIGS. 3A and 3B, with annotations showing waveform features representative of automated chest compressions and manual chest compressions. Specifically, as shown in FIGS. 3A and 3B, a compression depth (feature 901) is a measure of chest displacement as indicated by the peak to trough amplitude difference on a displacement waveform within a compression cycle. A compression rate (feature 902) is a number of compression cycles per unit time. A hold time (feature 903) is a time interval within the compression cycle between the downstroke and the successive upstroke. A velocity minimum-to-maximum time (feature 904) is the time interval on the velocity waveform from a velocity waveform trough to a successive velocity waveform peak within the compression cycle. A velocity amplitude (feature 905) is the difference on the velocity waveform between the amplitude of a velocity waveform peak and the amplitude of a successive velocity waveform trough. A compression width (feature 906) is the time interval between the onset of a compression and the end of a compression (i.e., the time interval between the start of the downstroke and the end of the upstroke for the compression cycle). A relaxation time (feature 907) is the time interval between compression cycles (i.e., the time interval between the end of the upstroke of a first compression cycle and the start of the downstroke for a second, successive compression cycle). A release time (i.e., the decompression time) (feature 908) is the time interval from the beginning to the end of an upstroke. Features 901, 902, 903, 906, 907, and 908 are indicated on the velocity waveforms in FIGS. 3A and 3B as illustrative examples. The computing device 116 can be configured to evaluate these features on one or more of the displacement waveform, the velocity waveform, and the acceleration waveform. The computing device 116 may select the particular waveform for evaluation based on the clarity of the features in the selected waveform as compared to the other waveforms and/or as compared to signal noise.

The features 901, 902, 903, 906, 907, and 908 illustrated in FIGS. 3A and 3B can have values or ranges of values representative of manual chest compressions and values or ranges of values representative of automated chest compressions. Accordingly, the computing device 116 of the system 100 can distinguish between manual chest compressions and automated chest compressions in the compression signals based on whether certain compression parameters or feature values are within the target range for manual chest compressions or within the target range for automated chest compressions. Non-limiting examples of values and value ranges for the features discussed above for manual chest compressions and for chest compressions by a belt-based automated chest compressor (as shown in FIG. 2B), are shown below in Table 1. However, the values in Table 1 are only meant as exemplary values for certain types of chest compressions performed by particular rescuers and compressor devices. Other values may need to be experimentally determined for other types of chest compressors or rescue conditions.

TABLE 1
COMPRESSION WAVEFORM FEATURES

| | | |
|---|---|---|
| Compression Type | Belt-based | Manual |

| | | | | |
|---|---|---|---|---|
| Compression Rate (901) | 77-83 | cpm | <206 | cpm |

| | | |
|---|---|---|
| Compression Depth (902) | 1-6 inches | 0.33-7 inches |
| | (2.5-15 cm) | (0.84-17.7 cm) |

| | | | | |
|---|---|---|---|---|
| Hold Time (903) | ≥120 | msec | ≤600 | msec |

| | | | |
|---|---|---|---|
| Velocity Minimum-to- | 120-480 | msec | Not evaluated |
| Maximum Time (904) | | | |

| | | |
|---|---|---|
| Velocity Amplitude (905) | >295 | 250-10000 |

| | | | | |
|---|---|---|---|---|
| Compression Width (906) | <562.5 | msec | 30-1300 | msec |

| | | | |
|---|---|---|---|
| Relaxation Time (907) | >300 | msec | Not evaluated |

| | | | |
|---|---|---|---|
| Release Time (908) | Not evaluated | ≤800 | msec |

Rescue Effort Timelines Generated from Case File Data

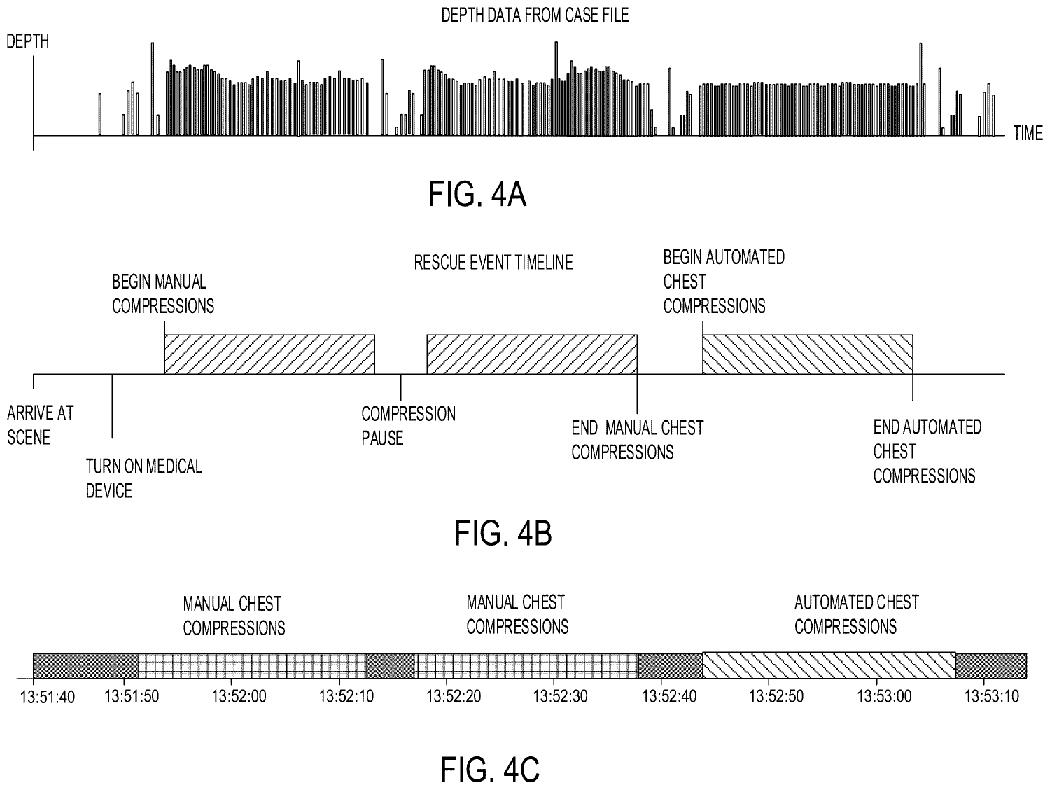

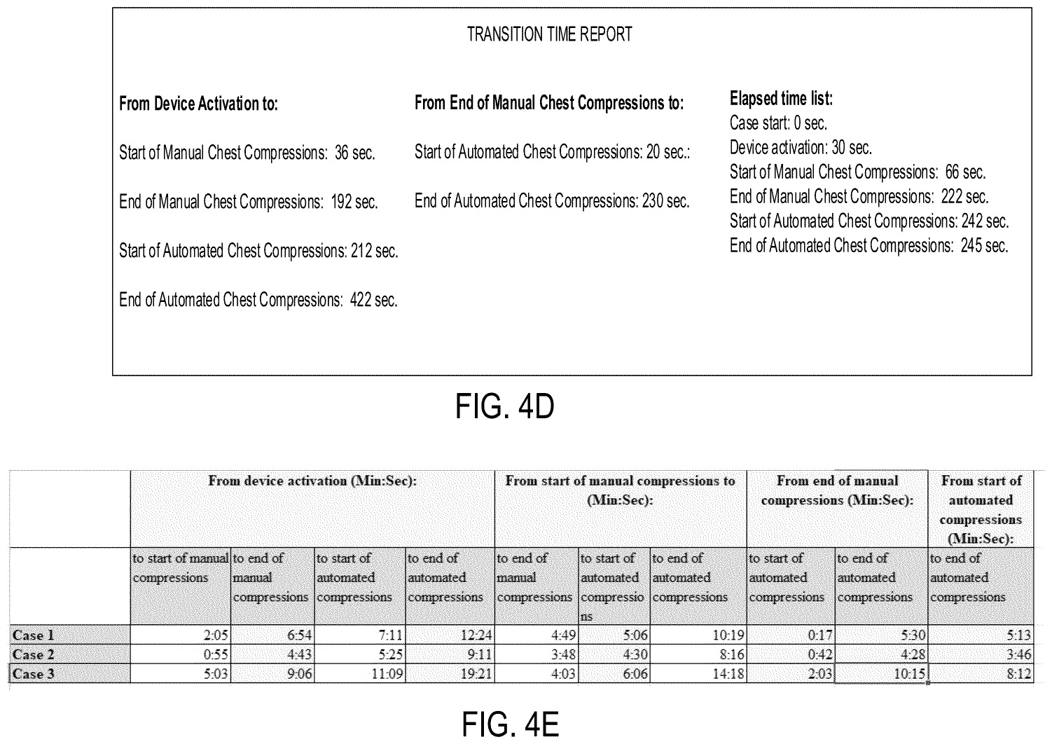

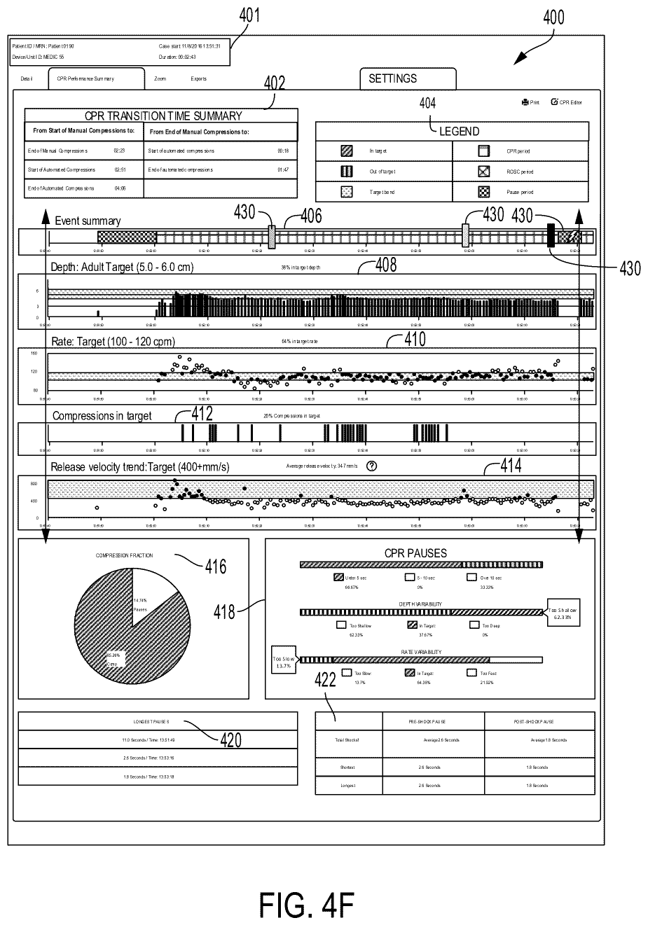

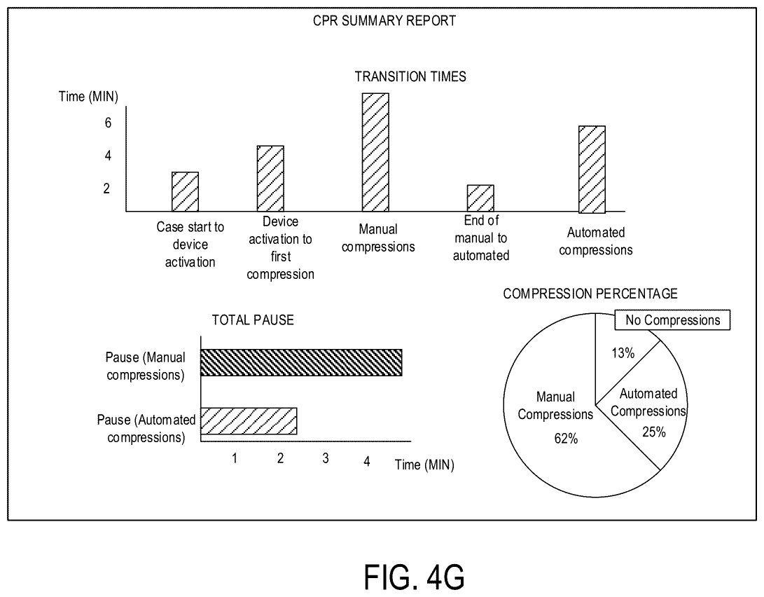

The medical device 112 may process signals from the chest compression sensor to generate timelines of events that occur during a rescue effort. For example, the medical device 112 may process the signals to identify individual chest compressions, which are represented by bars shown in FIG. 4A. The processing may include a mathematical integration of acceleration waveforms to determine a displacement waveform. The displacement waveform is representative of compression depth. As shown in FIG. 4A, some of the groups of bars are irregular having varying heights, meaning that the compressions varied in depth. Compressions of varying depth and/or rate are likely representative of manual chest compressions. By contrast, compressions having a consistent depth and/or rate are likely representative of automated chest compressions by a chest compressor, such as chest compressors shown in FIGS. 2B and 2C. In some cases, portions of signals from the chest compression sensor may include signal noise or artifacts caused by irregular movements that are not representative of either manual or automated chest compression. The computing device 116 can be configured to filter out or ignore signal artifacts from noise or non-chest compression movements.

The displacement or compression depth graph of FIG. 4A can be further simplified according to embodiments described herein where otherwise noisy signals are processed so as to identify and mark the occurrence of particular transition events, to provide a discrete timeline of events or activities that occur during a rescue effort. An exemplary event timeline is shown in FIG. 4B, which is annotated to show events including arrival of the rescuers at the rescue scene, turning on the medical device, start of manual chest compressions, pauses in manual chest compressions, such as pauses that occur during ventilation of the patient or ECG shock analysis to determine whether the patient should be treated with defibrillation, the end of manual chest compressions, the start of automated chest compressions, and the end of automated chest compressions. Times of occurrence of the identified events can also be recorded creating a time-stamped record of the rescue effort, which can be included in the case file for the rescue effort. A time stamped representation of the rescue effort showing the device turn-on time for the medical device and the start and end times for the manual and automated chest compressions is shown in FIG. 4C.

As previously described, the computing device 116 and/or reporting engine 162 of the system 100 are configured to generate the reports comprising the transition time indications for review by system users. The reports can be viewed on a display of the computing device 116, such as a display of a medical device 112 or local portable computing device at a rescue scene or a display of a remote computing device (e.g., a remote computing device and/or a relevant portal or console) at another location. Exemplary transition time reports are shown in FIGS. 4D-4E. As shown in FIG. 4D, a transition time report can comprise a list of transition times measured from a time of activation. The report also comprises a list of transition times measured from the end of manual compressions. The report also includes an elapsed time list, showing the elapsed time from, for example, case start to (i) device activation, (ii) start of manual compressions, (iii) end of manual compressions, (iv) start of automated compressions, and (v) end of automated compressions. FIG. 4E is a table of transition time values, which can be included, for example, in a final case report for a rescue effort. Further, as shown in FIG. 4E, the table can comprise transition time values for multiple cases or rescue efforts, which can allow rescuers to compare transition times between different rescue efforts.

FIG. 4F is a graphical user interface dashboard (GUI dashboard 400) which can be displayed, for example, on a website, and which can be accessed by a user using a personal computer, smart phone, computer tablet, or similar electronic device. Exemplary GUI screens, which can be generated by the systems 100 of the present disclosure including information about events occurring during a rescue effort are described, for example, in U.S. Pat. No. 11,033,455, entitled “Tools for case review performance analysis and trending of treatment metrics,” which is incorporated herein by reference in its entirety. The reporting engine 162 (shown in FIGS. 1A and 1B) may generate the user interface dashboard (e.g., the dashboard 400).

With reference to FIG. 4F, the dashboard 400 comprises a CPR Transition Time Summary 402 including transition time values for time from device activation, time from start of manual compressions, and start of automated chest compressions. The dashboard 400 also comprises a dashboard summary 401, which can include a patient identification or other medical record number, as well as identifying information about one or more devices at a rescue scene and/or one or more caregivers who provided care for the patient. The dashboard summary 401 can also comprise information about a caregiver organization used during the case, as well as the date and time when the case began and the duration of the case. The dashboard 400 can also comprise an event summary 406, which provides a high-level overview of the case, conveying to the user information about events (e.g., CPR activities) that occurred over the course of the case. The event summary 406 can comprise, for example, a chest compression depth summary 408, a chest compression rate summary 410, a compressions-in-target summary 412, and/or a release velocity trend summary 414, according to embodiments of the present disclosure. The event summary 406 can also comprise event markers 430, which are icons showing a time during the rescue effort that particular events occurred. For example, event markers 430 can indicate times of occurrence for events including device activation, start of manual and/or automated chest compressions, end of manual and/or automated chest compressions, cardiac events (e.g., a cardiac arrest detected or heart attack detected), or treatment events (e.g., drug administration or patient shock), as well as other information about the patient or rescue effort.

In some examples, the dashboard 400 may further comprise a compression fraction summary 416 indicating a percentage of time when compressions were being applied over the case versus a percentage of time when the caregiver was pausing. Also, the dashboard 400 may further comprise a CPR pause length summary chart 418 that indicates, for example, relative length of chest compression pauses in color code (e.g., under 5 seconds, 5-10 seconds, and over 10 seconds), depth variability in color code (e.g., too shallow, in target, and too deep), and rate variability in color code (e.g., too slow, in target, and too fast). Long pauses in chest compressions may lead to poorer outcomes for patients in cardiac arrest. The CPR pause length summary chart 418 can show information about pauses and how long they were. The depth variability chart shows in percentages how well the caregivers did performing chest compressions related to the depth they achieved. The rate variability summary shows in percentages how well the caregivers did performing chest compressions at the target rate. The longest pauses summary 420 may indicate the length of the longest pauses and the time(s) during the event when they occurred. A pre-shock/post-shock summary 422 may indicate the total number of shocks, as well as the average, shortest, and longest pause both pre-shock and post-shock, according to embodiments of the present disclosure. In other words, the longest pauses summary 420 shows the three longest pauses that were detected during the case. It shows how long the pause was and at what time during the event it occurred. In some examples, clicking on or otherwise selecting the display of one or more pauses navigates the user to display a portion of the case that contains the long pause. Similar navigation options are also possible for selecting pre- or post-shock pauses. The pre-shock/post-shock summary 422 shows, for example, the total number of shocks delivered during a case and how long chest compressions were paused immediately before and after the shock.

FIG. 4G shows another CPR Summary Report for transition time information determined by the system 100. The CPR Summary Report includes visual indications for determined or calculated transition time values. For example, the CPR Summary Report comprises a number of transition time indications, for example, a transition time graph with bars showing different transition times during the rescue effort (e.g., the time from case start to device activation, time from device activation to first compression, time from start to end of manual compressions, time from end of manual compressions to start of automated compressions, and time from start to end of automated compressions). The CPR Summary Report also includes a transition time indication, for example, a bar chart showing total pause during manual compressions and total pause during automated compressions so that users can compare how often compressions were interrupted during manual and automated chest compression cycles. The CPR Summary report also includes a transition time indication, for example, a pie chart with segments for time spent for manual compressions, automated compressions, and no compressions during the rescue effort.

### Compression Signal and Case File Processing Methods

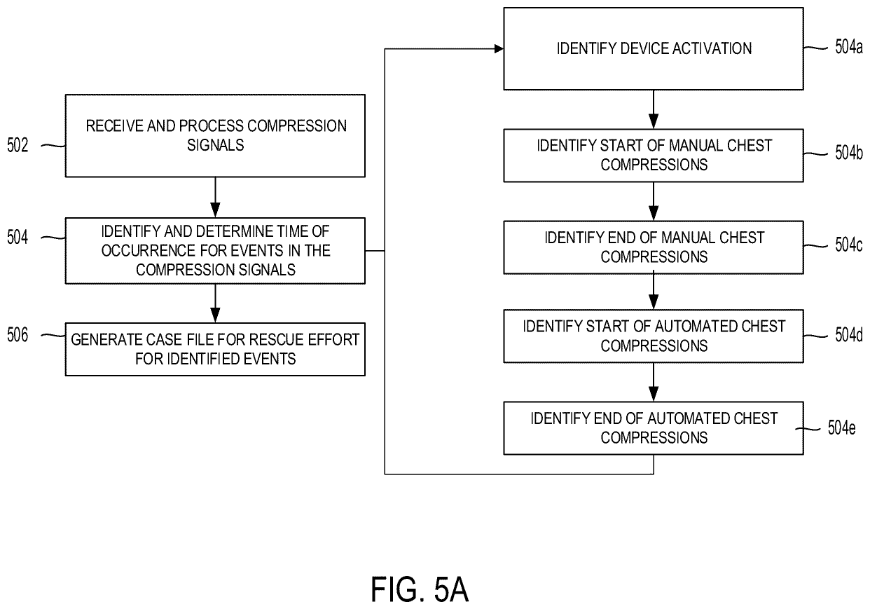

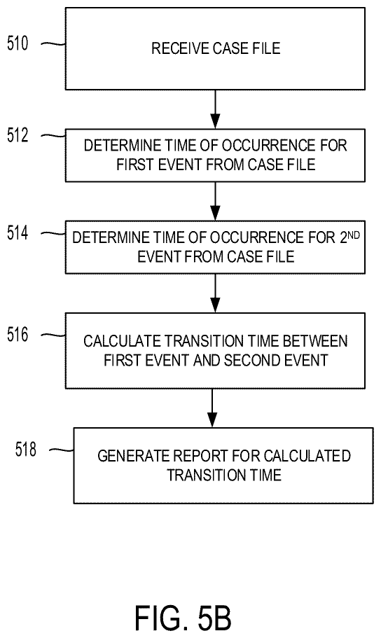

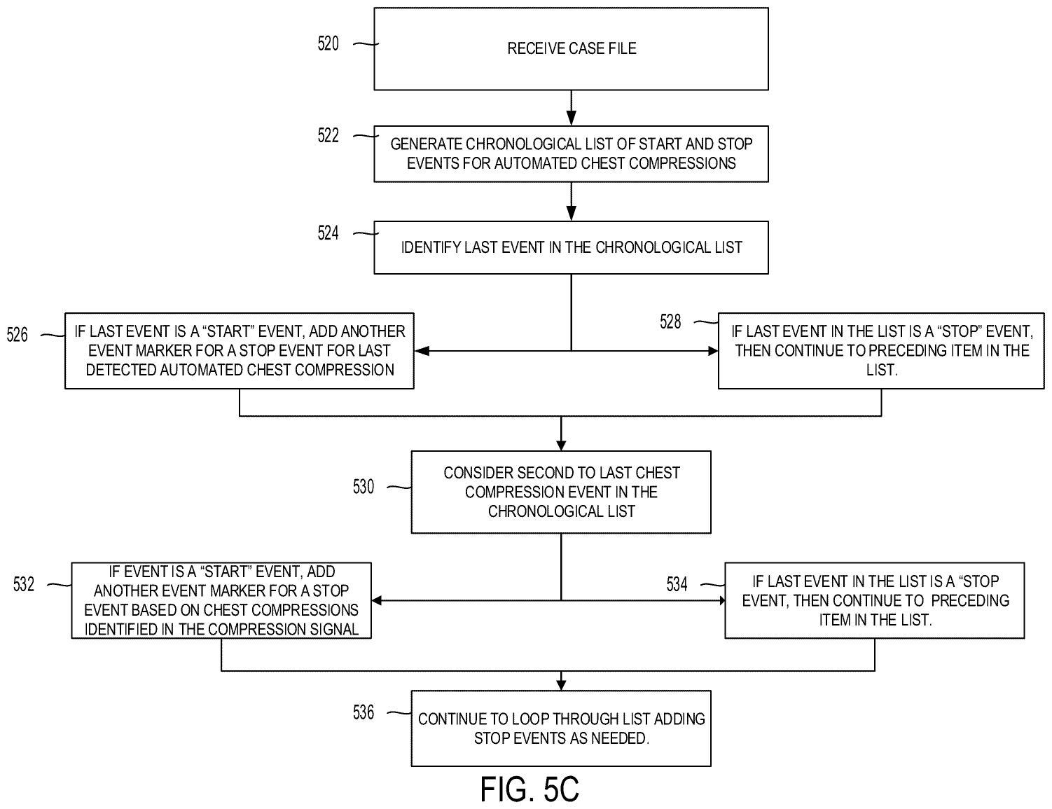

FIGS. 5A and 5B are flow charts showing processes or computer implemented methods for generating the case file and the transition time report. For example, the medical device 112 may perform the method of FIG. 5A to generate the case file and the medical device 112, the computing device 116, and/or reporting engine 162 can perform the method 5B to generate the transition time report.

As shown in FIG. 5A, a method for generating the case file can comprise, at step 502, receiving the time-correlated signals representative of chest compressions performed for the patient. The method further comprises, at step 504, identifying and determining a time of occurrence for events in the received and processed compression signals. For example, events comprise device activation (step 504a), start of manual chest compressions (step 504b), end of manual compressions (step 504c), start of automated chest compressions (step 504d), or end of automated compressions (step 504e) can be identified in the received and processed time-correlated signals.

More specifically, in some examples, a start of manual compressions can be identified by generating data representation of a compression waveform from the received and processed time-correlated signals and identifying portions of the compression waveform representative of manual chest compressions provided for the patient. Specifically, the portions representative of manual chest compressions can be identified by calculating a chest compression parameter value for multiple segments of the compression waveform; comparing the calculated chest compression parameter value for the multiple segments to a target range for the chest compression parameter values representative of manual chest compressions; and identifying segments of the compression waveform with the chest compression parameter value within the target range. The chest compression parameters can include, for example, compression rate, compression depth, compression hold time, variation in compression rate, variation in compression depth, variation in hold time, compression width, relaxation time, release time, compression average velocity, compression maximum velocity, or velocity minimum to maximum time (per chest compression cycle), as well as any of the features shown in FIGS. 3A and 3B, described previously. To verify that the start of manual compressions indeed has occurred with sufficient sensitivity, for some embodiments, it may be required that such parameter values be within specified ranges over a period of time and/or number of identified compressions.

Once the portions of the waveform representative of manual and/or automated chest compressions are identified, the start of manual chest compressions can be identified by determining a first or earliest time of the portions of the compression waveform representative of the manual chest compressions. In a similar manner, the end of manual compressions can be identified by determining a final or latest time of the portions of the compression waveform representative of the manual chest compressions. The start of automated chest compressions and the end of automated chest compressions can be identified in a similar manner. Specifically, the start of automated chest compressions can be identified by identifying portions of the compression waveform representative of automated chest compressions and determining a first time represented in the automated chest compression portions.

The method can further comprise, at step 506, generating the case file for the rescue effort, which includes times of occurrence for a plurality of medical events. For example, the generated case file can comprise a time-stamped record of the events occurring during the rescue effort. In some examples, the generated case file can also include the signals generated.

FIG. 5B is a flow chart illustrating a method for generating the report from the case file. As previously described, the processing steps of FIG. 5B can be performed by the medical device 112, the computing device 116, and/or the reporting engine 162. The method comprises, at step 510, receiving the case file for the rescue effort from the at least one medical device. For example, the case may be received by the reporting engine 162 from the medical device 112. At step 512, the method further comprises selecting and determining the time of occurrence for a first event of the plurality of medical events from the case file. The method further comprises, at step 514, selecting and determining the time of occurrence for a second event of the plurality of medical events from the case file occurring after the selected at least one first event. The method further comprises at step 516, determining or calculating a transition time between the time of occurrence of the at least one first event and the time of occurrence of the at least one second event. The method can further comprise, at step 518, generating the report that provides a transition time indication representative of the determined or calculated transition time for user review. As previously described, the generated report can also include other information about the rescue effort including, for example, patient physiological information and/or information about patient outcomes, such as whether there was a return of spontaneous circulation (ROSC) and/or whether cardiac arrest or heart attack events were detected during the rescue effort.

In some examples, the generated case file may not include both a start time and an end time for each occurrence of manual and/or automated chest compressions. This may occur, for example, if the received time-correlated compression sensor signal is noisy or includes movement artifacts that obscure a start or end for a particular group of automated or manual chest compressions. Therefore, in order to determine or calculate transition times for certain chest compression events in the case file, it may be necessary add certain event markers to the case file so that each grouping of compressions has a start time and a matching stop time. In various embodiments, this generation and/or adding of event markers to the case file can be performed, for example, by the medical device 112, computing device 116, and/or by the reporting engine 162. In various implementations, the computing device 116 and/or reporting engine 162 may analyze the received case file data to add event markers including compression cycle event markers to the case file data and generate a report and GUI based on these additional event markers. The reporting engine 162 may provide the only source of the compression cycle event markers (e.g., if the medical device does not identify compression cycles and/or types of compressions associated with the compression cycles) or the reporting engine 162 may supplement compression cycle event markers provided in the case file by the medical device. The ability of the reporting engine 162 to supplement event markers may provide redundancy to improve the accuracy of the reported data.

In an implementation, the reporting engine 162 may apply a change-point analysis to one or more of the compression depth and compression rate data reported as a function of time in the case file. For a change-point analysis, the reporting engine 162 may analyze a set of time-ordered data (e.g., the compression depth and/or compression rate data) during a time period after which the data is collected (e.g., for case data, the analysis occurs post-case). In a change-point analysis, a quantity derived from the time-order data (for example, a cumulative sum) may be determined as a function of time for the set of data and changes in slope for the derived quantity plotted as a function of time may be analyzed to find change points. The change points may correspond to transitions between compression periods and pause periods and/or transition between manual compression periods and automated compression periods. The reporting engine 162 may add event markers to the report based on the identified change points. This analysis may include analyzing additional derived quantities, reordering data, determining statistical measures, etc. When changes are detected, data segments on either side of the change may be further analyzed to verify the detected changes and to determine if additional changes occur within these data segments. The change point analysis may generate an indication of a confidence level in the detected change. For example, the change point analysis may indicate a particular percent of confidence that a detected change actually occurred. The change point analysis may be applied to collected data points and/or to statistical measures of the data points (e.g., averages, standard deviations, ranges, etc.

Additionally or alternatively, in an implementation, the reporting engine 162 may add event markers according to the steps shown in the flow chart of FIG. 5C. This flow chart shows steps for adding event markers to a case file so that identified groups of automated chest compressions have both a start and stop time is shown in FIG. 5C. A similar process may be followed for providing any missing event markers for manual chest compressions.

As shown in FIG. 5C, at step 520, a case file including the start and stop events for groupings of automated chest compressions is received. At step 522, a chronological list of start and stop times for chest compression events (e.g., begin compression and end compression for the automated chest compressions in the case file) is generated from information in the case file. At step 524, the last automated chest compression event in the chronological list is identified. At step 526, if the last automated chest compression event in the list is a “start” event without a matching stop event, meaning that an anomaly in the list has been identified, the method further comprises finding a timestamp in the case file data for a final automated chest compression provided to the patient occurring after the last “start” event in the chronological list. At step 526, an event marker is then added for an end or stop of the automated compressions at the time for the final automated chest compression, as indicated by the timestamp. At step 528, if the last automated chest compression event in the chronological list is a “stop” event, then the final event is correct and no modification to the list is needed and no additional events are added.

Next at step 530, the second to last automated chest compression event in the chronological list is considered. If the second to last event is a “stop” event, then no adjustment to the list is needed (step 534). If the second to last automated chest compression event in the list is a “start” event without a matching stop event (step 532), the method further comprises finding a timestamp in the case file data for an automated chest compression provided to the patient occurring after the send to “start” event being considered. An event marker is then added for an end or stop of the automated compressions at the time for the final automated chest compression, as indicated by the timestamp.

At step 536, the method comprises continuing to loop through the chronological list of events from the case file to ensure that each automated chest compression “start” event is matched with a corresponding “end” event. If a “start” event cannot be matched with a corresponding “end” event, then the method comprises adding an event marker for an “end” event at a point in time after the unmatched “start” event. As previously described, the time for the added “end” event is based on a timestamp for an automated chest compression determined from the chest compression sensor signal occurring after the unmatched start event. The looping is continued until all automated chest compression “start” events in the case file or chronological list are matched with a corresponding chest compression “stop” or end event.

### User Interface Screens for Chest Compression Review

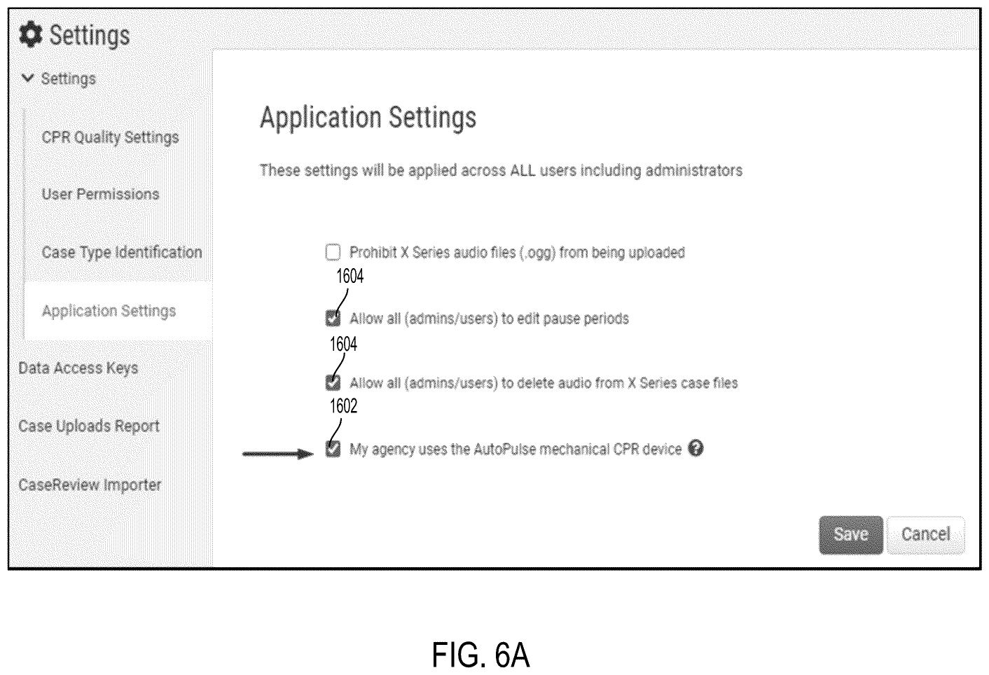

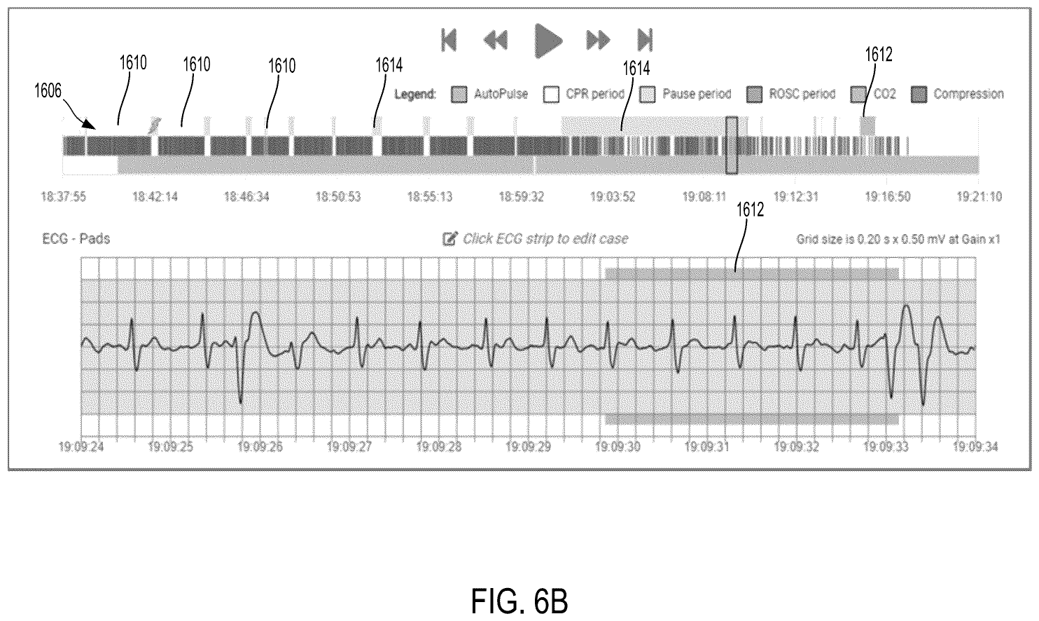

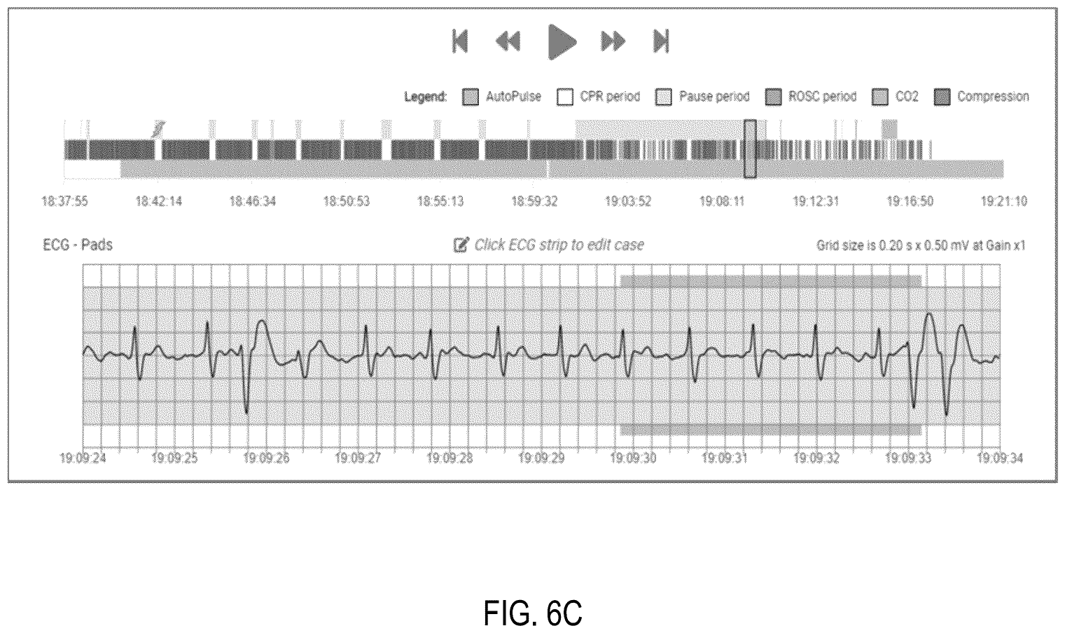

FIGS. 6A-8B show examples of user interface screens (referred to herein as UI screens) that can be displayed to a user to provide simplified representations that condense data collected during rescue effort(s) and/or included in the case file(s) for one or more rescue efforts. The UI screens help users to evaluate chest compression data collected by the previously described systems, such as the systems 100 shown in FIGS. 1A-1C. The UI screens can be displayed on a computing device, such as a medical device or local portable computing device at a rescue scene. The UI screens can also be displayed on remote computing devices, such as a technician terminal or another relevant portal or console, or on a user's personal computer. For example, the UI screens can be accessed by a user on a webpage hosted on a server, such as the servers shown in FIGS. 1A and 1B. The reporting engine 162 (shown in FIGS. 1A and 1B) may generate the UI screens exemplified in FIGS. 6A-8B.

The UI screens allow a user to identify cases or rescue efforts where a mechanical chest compressor (e.g., AutoPulse) for providing automated chest compressions to a patient was used or was available for use. For example, a user may select a virtual button or icon on an initial UI screen to indicate that the chest compression (e.g., AutoPulse) was in use or available. In this particular embodiment, when the AutoPulse box is selected, the system 100 can be configured to analyze a case file or rescue effort data to provide information for user review about the automated chest compressions provided to the patient and/or comparing automated chest compression to manual chest compressions provided to the patient.

For example, when the AutoPulse box is selected, the UI screens can include CPR breakout boxes or modules, providing separate statistics for manual chest compressions, automated chest compressions, and total chest compressions provided for a patient. The UI screens can also provide shading or other visual indications on timelines and waveforms for the rescue effort, such as a timeline showing individual chest compressions, so that users can distinguish between portions of the timeline from manual chest compressions and portions of the timeline from automated chest compressions. The UI screens can also include aggregated CPR trend information displays with data for manual chest compressions, automated chest compressions, and total chest compressions for multiple rescue efforts or multiple case files over a particular time period (e.g., weeks, months, years).

The UI screens can also include case management or filtering screens and functions. For example, a user may initially be provided with a list of cases occurring during a particular period of time (e.g., cases occurring during a particular day, week, or month). The user may search or filter the list of cases to see which cases include automated chest compressions and/or to identify cases where a mechanical chest compressor was available. Case management screens including lists of cases may also include icons or indicators for AutoPulse cases allowing users to easily identify cases where automated chest compressions (AutoPulse) were performed. Users may also compare outcome information for the listed cases to compare outcomes for cases with automated chest compressions to outcomes for cases without automated chest compressions.

FIG. 6A shows an initial UI screen, where a user can identify that the user's agency uses a mechanical chest compressor device, such as AutoPulse, by checking an appropriate box 1602. As previously described, when AutoPulse is selected, the system can be configured to evaluate received case files to provide information about the automated compressions provided for the patient. As shown in FIG. 6A, the user may also select boxes 1604 related to administrative permissions, such as allowing users to edit graphs or waveforms and/or to delete audio data from a case file.

FIG. 6B is a UI screen that allows a user to review a timeline including chest compression events and ECG strip data collected during the rescue effort. The UI screen includes a chest compression timeline 1606 including bars representing each compression provided during the rescue effort. The timeline also includes shading (shown by reference numbers 1610, 1612, 1614) to distinguish between manual compressions and automated compressions. Specifically, shading 1610 represents manual compressions, shading 1612 represents automated chest compressions, and shading 1614 represents periods of time when no chest compressions are provided. Shading (as shown by shading 1612 in FIG. 6B) can also be provided proximate to the ECG trace to show types of chest compressions performed during specific segments of the ECG trace. The timeline also includes sections that indicate compression pause periods, ROSC periods, and also the time in which a CO₂ sensor was applied for patient monitoring. The UI screen also includes virtual buttons, such as play, fast forward, and rewind buttons, that allow the user to review data for different periods of time during the rescue effort represented by the case file.

FIG. 6C shows another UI screen including the ECG waveform. The UI screen includes an annotation or marker toolbar allowing a user to annotate the ECG strip to show events occurring during the rescue effort, such as adding an annotated ECG strip to show when automated chest compressions (AutoPulse) started or ended.

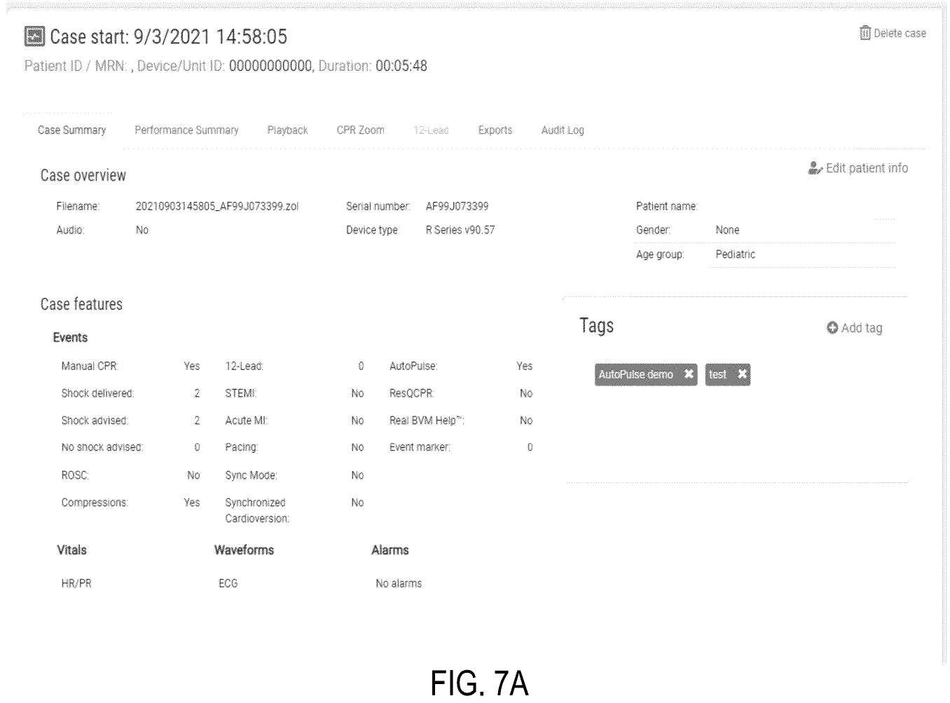

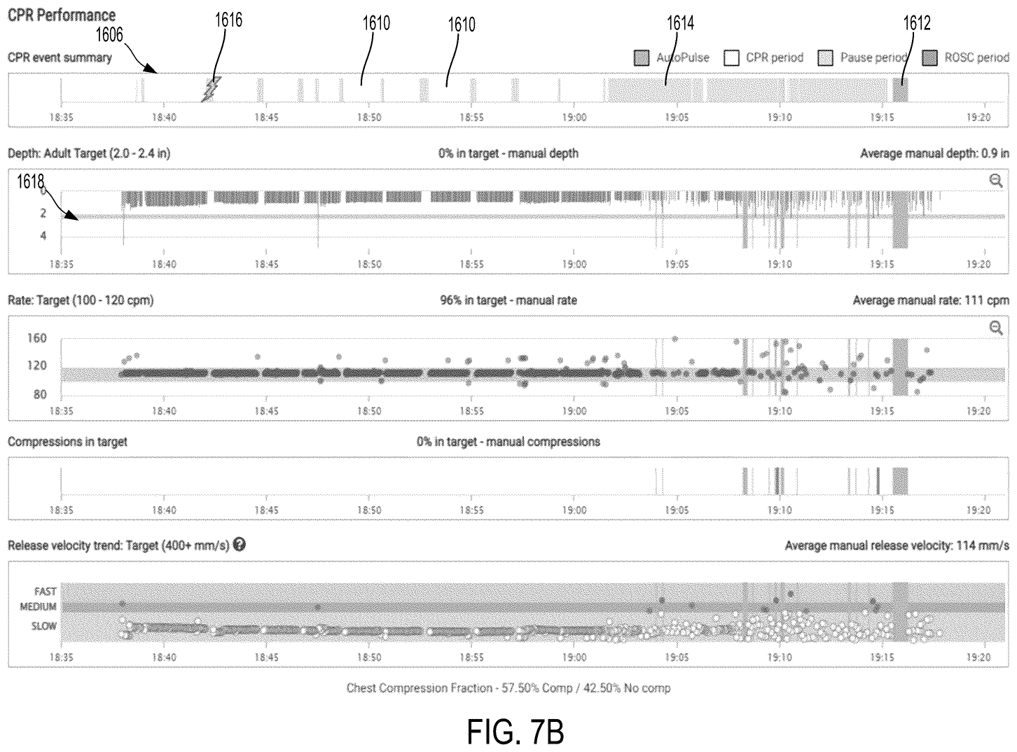

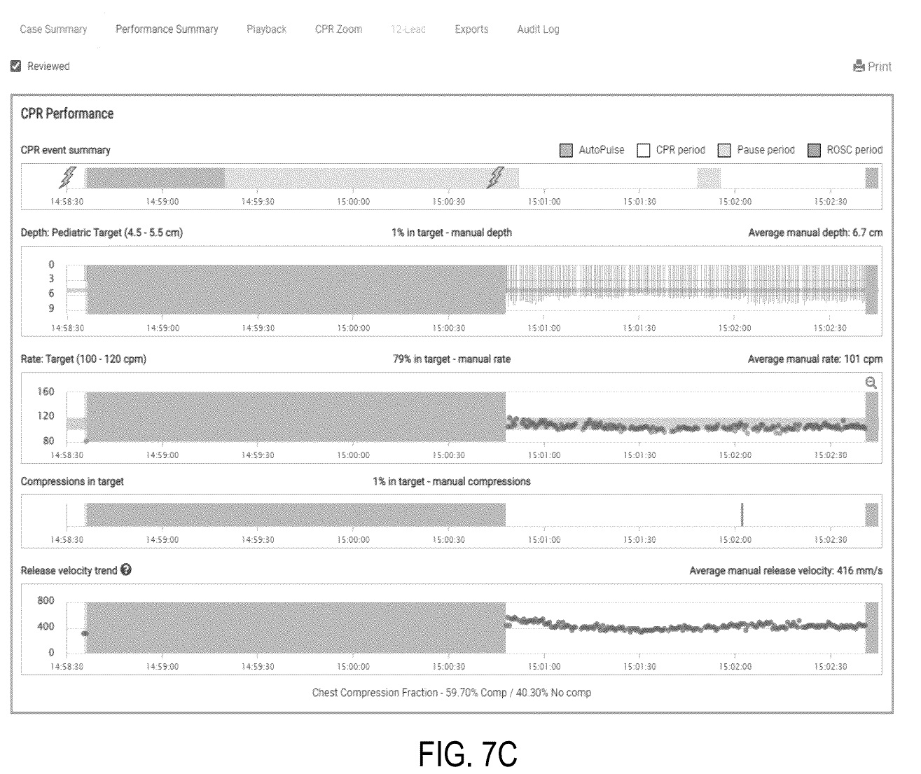

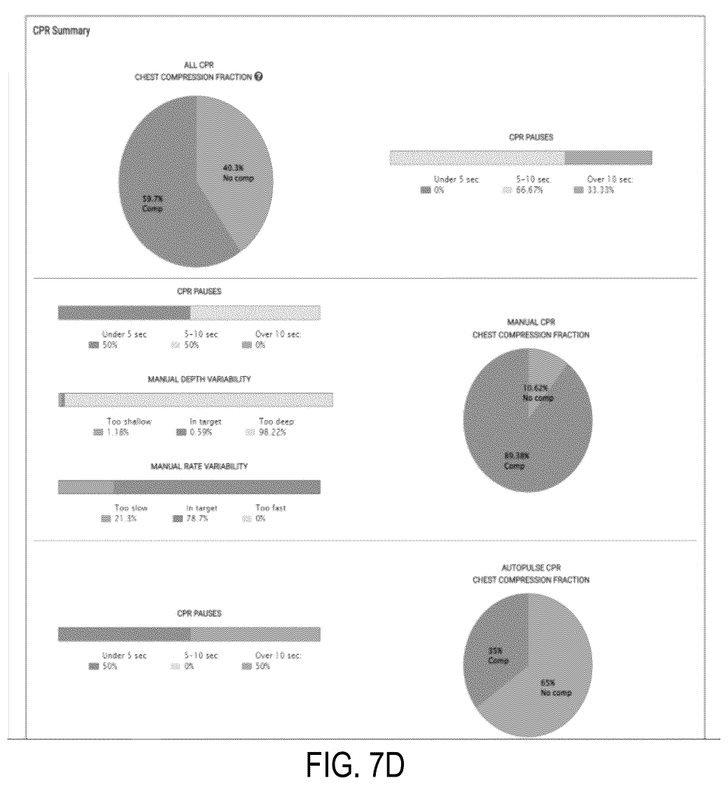

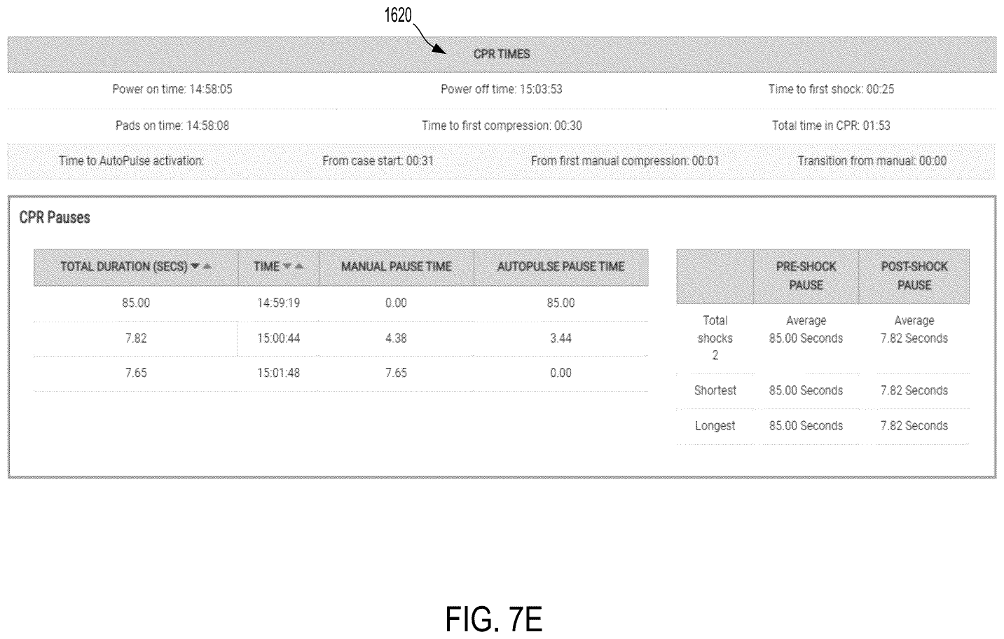

FIGS. 7A-7E show another sequence of UI screens that can be provided to users for reviewing case files generated during a rescue effort. FIG. 7A is an initial UI screen showing case overview information (e.g., file name, medical device type, and patient name), and case feature information. For example, the case feature information can include information about types of chest compressions performed during the case (e.g., manual compressions, automated compressions, or compressions using a ResQCPR® device). The screen can also show information about cardiac treatments provided (e.g., shocks delivered, shocks advised, pacing, synchronized cardioversion, etc.) and/or cardiac events detected (e.g., ROSC, STEMI, etc.). The case features may also include information about types of devices or device features used during the rescue effort, such as whether a 12 lead ECG was collected or whether a ventilation guidance (BVM help) feature was used during the rescue effort.

FIG. 7B is a UI screen showing CPR performance information in a case file. Specifically, the screen includes a CPR event summary timeline 1606 with icons showing events, such as a patient shock (shown by icon 1616), occurring during the rescue effort. The timeline can also include shading showing times during the rescue event when manual chest compressions were provided (shown by shading 1610), automated chest compressions were provided (shown by shading 1612), or no chest compressions were provided (shown by shading 1614). As shown by the shading 1610, 1612, 1614 on the timeline 1606, the rescue event documented by the case file included a period of manual chest compressions with intermittent pauses. After the end of the manual chest compressions, there was a long period of pause (e.g., the transition time between the end of manual chest compressions and the start of automated chest compressions). After the long period of pause, there was a period of automated chest compressions.

The UI screen of FIG. 7B also shows graphs and timelines showing compression depth, compression rate, and release velocity for chest compressions performed for the patient. The graphs and timelines can include icons, shading, highlighting, or other visual indicators showing whether particular compressions were within or outside of a target range for the chest compressions for an adult patient. For example, the shaded line 1618 in FIG. 7B shows a target compression depth of 2.0 in. Bars that are above the target line 1618 represent shall compressions that did not reach the target depth. Bars that extend below the target line 1618 represent chest compressions that were either within the target range or too deep. FIG. 7C shows another UI screen showing CPR performance information. In FIG. 7C, the target icons and other visual indicators show target values for a pediatric patient.

FIG. 7D is a UI screen showing a CPR summary screen comprising chest compression statistics broken up for manual chest compressions, automated chest compressions, and total chest compressions. In particular, the UI screen shows graphs illustrating CPR pause for manual, automated, and total compressions. The UI screen also includes pie charts showing compression fraction for manual, automated, and total compressions. The UI screen also includes indications for manual depth variability and manual rate variability.

FIG. 7E shows another portion of a UI screen with CPR summary information. Specifically, the UI screen shows transition time information for a rescue effort generated from a case file. For example, as shown in FIG. 7E, the UI screen includes a table of transition time information (CPR Time table 1620). The table 1620 includes a power on time, power off time, time to first shock, pads on time, time to time to first compression, and total time in CPR. The table 1620 also includes values for a time to AutoPulse activation: from case start, from first manual compression, and transition from manual compression to AutoPulse. The UI screen also shows information about pauses during both manual and automated chest compressions. Also, the UI screen includes information about post-shock pause and pre-shock pause.

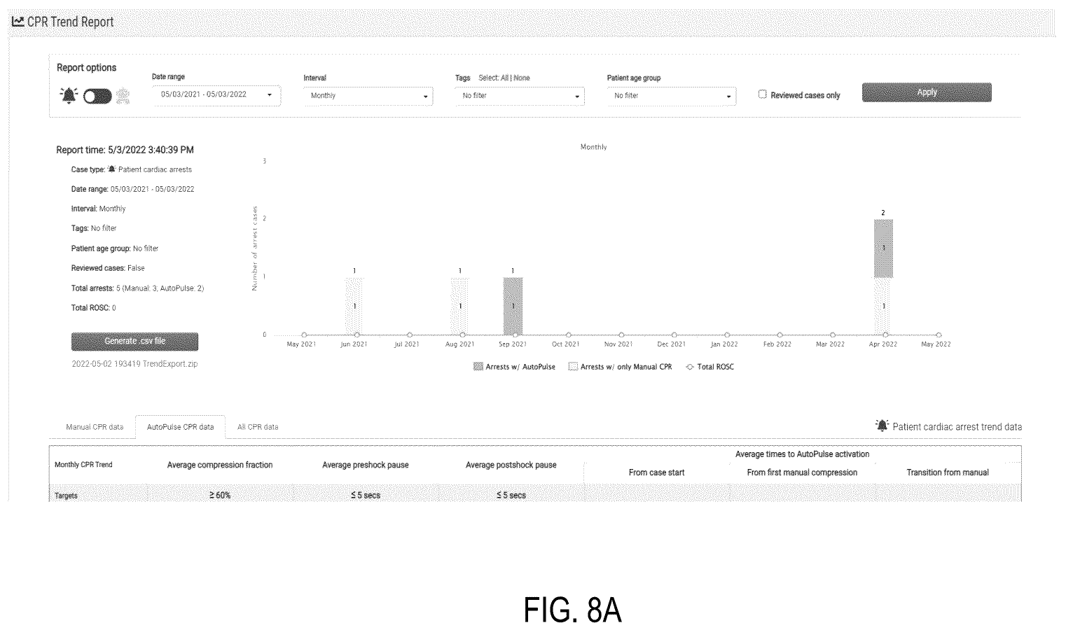

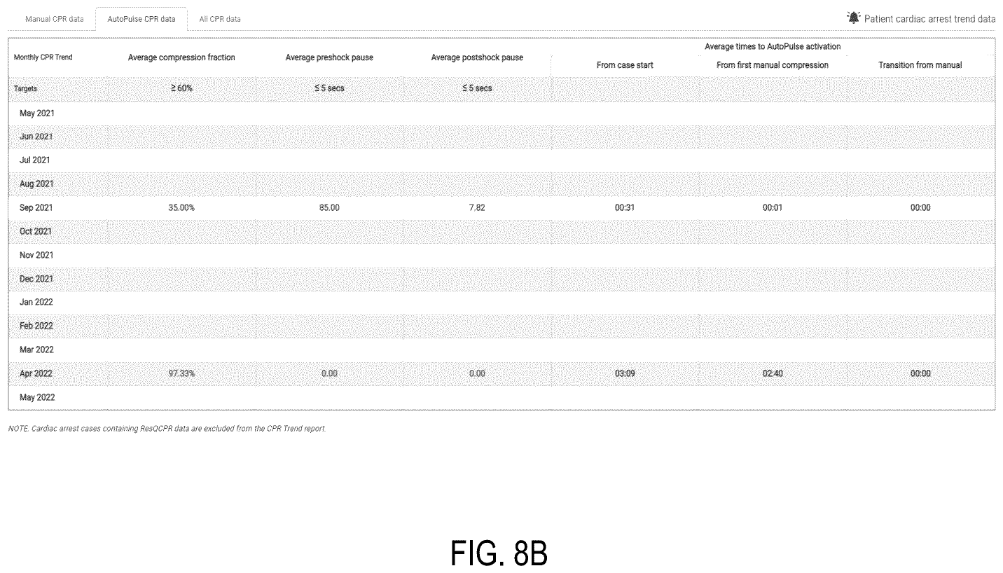

FIGS. 8A and 8B show CPR statistics for multiple case files and/or multiple rescue efforts. For example, as shown in FIG. 8A, the UI screen includes a bar graph with bars showing a number of arrest cases per month. The bars can be color-coded to distinguish between cases where only manual compressions were performed and cases where both manual and automated compressions were performed. FIG. 8B is a table showing numerical values for CPR statistics for the multiple case files and/or rescue efforts. The table is divided by month. The table includes average CPR statistics for multiple rescue efforts. The table also includes transition time trend information, such as the average time to automated chest compressions (e.g., AutoPulse) (i) from case start, (ii) from first manual compressions, and (iii) a transition time from manual compressions to automated compressions.

### Ventilation Monitoring and Transition Time Reporting

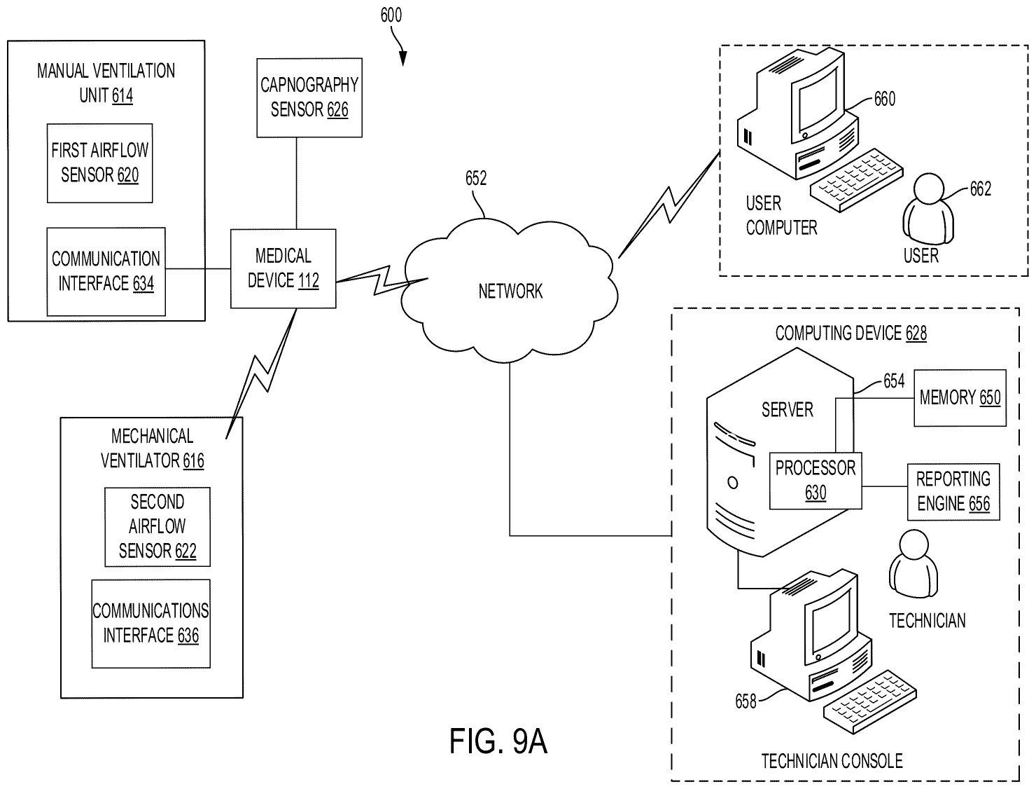

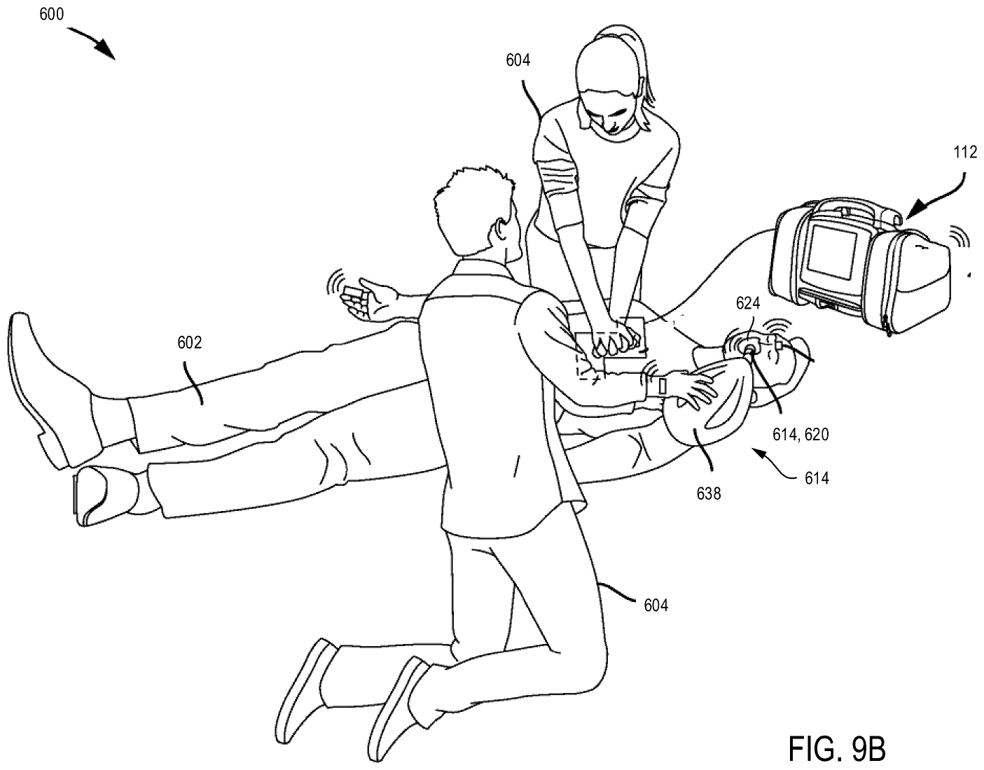

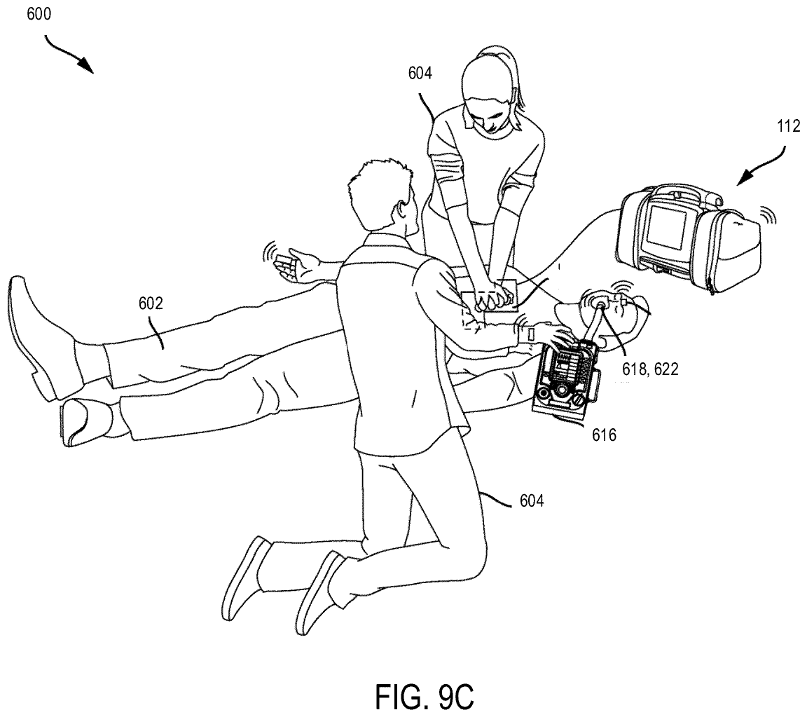

FIGS. 9A-9C illustrate components of a system 600 for monitoring and/or reviewing transitions between types of ventilations provided to a patient 602. For example, the transition times reported by the system 600 can be between events including: (i) activation of a ventilation unit and a start of manual ventilations, (ii) activation of the ventilation unit and an end of the manual ventilations, (iii) activation of the ventilation unit and a start of the mechanical ventilations, (iv) activation of the ventilation unit and an end of the mechanical ventilations, (v) the start of the manual ventilations and the end of the manual ventilations, (vi) the start of the manual ventilations and the start of the mechanical ventilations, (vii) the start of the manual ventilations and the end of the mechanical ventilations, (viii) the end of the manual ventilations and the start of the mechanical ventilations, (ix) the end of the manual ventilations and the end of the mechanical ventilations, and (x) the start of the mechanical ventilations and the end of the mechanical ventilations.

The system 600 comprises medical devices including a manual ventilation unit 614 and a mechanical ventilator 616. One or more of the medical devices can be connected to and/or in communication with other medical devices at the rescue scene, such as the defibrillator or patient monitor shown in FIGS. 1A-1C. The system 600 can also comprise an airflow path 618 (shown in FIGS. 9B and 9C) configured to be in fluid communication with an airway of the patient 602 for providing manual or mechanical ventilations to the patient 602. The airflow path 618 can comprise airflow sensors, such as a first airflow sensor 620 of the manual unit 614 or a second airflow sensor 622 of the mechanical ventilator 616, positioned to sense time-correlated signals representative of airflow in the patient's airway. The airflow path 618 can also comprise a mask 624 (shown in FIGS. 9B and 9C) that seals to and fits over a lower portion of a face of the patient 602 for providing airflow to the patient 602. In some examples, mechanical ventilations are provided to the patient 602 through an intubation tube. In some examples, the system 600 further comprises a capnography sensor 626 (shown in FIG. 9A), which can be connected to the airflow path 618 and can detect data representative of CO₂ from an exhaled breath of the patient 602.

In some examples, the manual ventilation unit 614 for providing manual ventilations to a patient comprises a flexible bag 638 (shown in FIG. 6B) configured to be connected to the airflow path 618 to provide manual ventilations for the patient 602. The bag 638 can be manipulated by one of the rescuers 604 for providing the manual ventilations to the patient 602. Optionally, manual chest compressions can be also be provided to the patient 602, as shown in FIG. 6B. The first airflow sensor 620 can be connected to the airflow path 618 for measuring the ventilation airflow from the bag 638 to the patient 602. The first airflow sensor 620 can be connected to the medical device 112, such as a patient monitor or defibrillator, for monitoring signals detected by the airflow sensor 620.

In a similar manner, the manual ventilator 616 is shown in FIG. 6C for providing airflow to the patient 602 through the airflow path 618. The mechanical ventilator 616 can be any mechanical ventilator 616, known in the art, for providing ventilations to the patient 602. The second airflow sensor 622 is positioned in the airflow path 618 between the mechanical ventilator 616 and the patient 602 for detecting ventilation airflow generated by the ventilator 616.

In some examples, in addition to determining the transition time(s), the system 600 can also be configured to determine and, in some cases, provided feedback about a quality of ventilation activities performed for the patient 602. For example, the medical device 112 (shown in FIGS. 1A-1C, 9B, and 9C) at the rescue scene can be configured to determine a ventilation rate for the patient 602 based on analysis of time-correlated signals from the airflow sensor 620, 622. The medical device 112 may also be configured to compare the determined ventilation rate to a target ventilation rate range and cause a ventilation rate indication to be displayed on a visual display, such as a display of the medical device 112, indicating whether the ventilation rate is within or outside of the target range. The ventilation rate range comprises a ventilation rate of about 10 ventilations per minute to about 20 ventilations per minute.

With continued reference to FIG. 9A, the system 600 further comprises a computing device 628 communicatively coupled with the airflow sensor(s) 620, 622 via, for example, a communication interface 634 of the manual ventilation unit 614 or a communication interface 636 of the mechanical ventilator 616. In some examples, the computing device 628 is remote from the rescue scene and receives data from the airflow sensor(s) 620, 622 transmitted from a medical device, such as the medical device 112 (shown in FIGS. 1A-1C, 9B, and 9C), at the rescue scene over a network 652.

In some examples, the computing device 628 can be a computer server 654 or another electronic device for processing data obtained by sensors, such as the airflow sensors 620, 622. The server 654 can comprise a processor 630, computer readable memory 650, and a reporting engine 656 for receiving the case file and generating the report. In particular, the reporting engine 656 can comprise hardware and software for processing received case file to determine transition times between events in the case file. The system 600 can also comprise a technician console 658 comprising a visual display for providing information about the ventilations performed for the patient 602 to a user, such as a technician using a technical console or portal. In other examples, the computing device 628 can be configured to transmit a generated report or other information about transition times to remote devices or networks to be viewed by users. For example, the generated report and/or other information can be transmitted from the server 654 of the computing device 628 to a user computer 660 for displaying the report and/or other information about a rescue effort to a user 662.

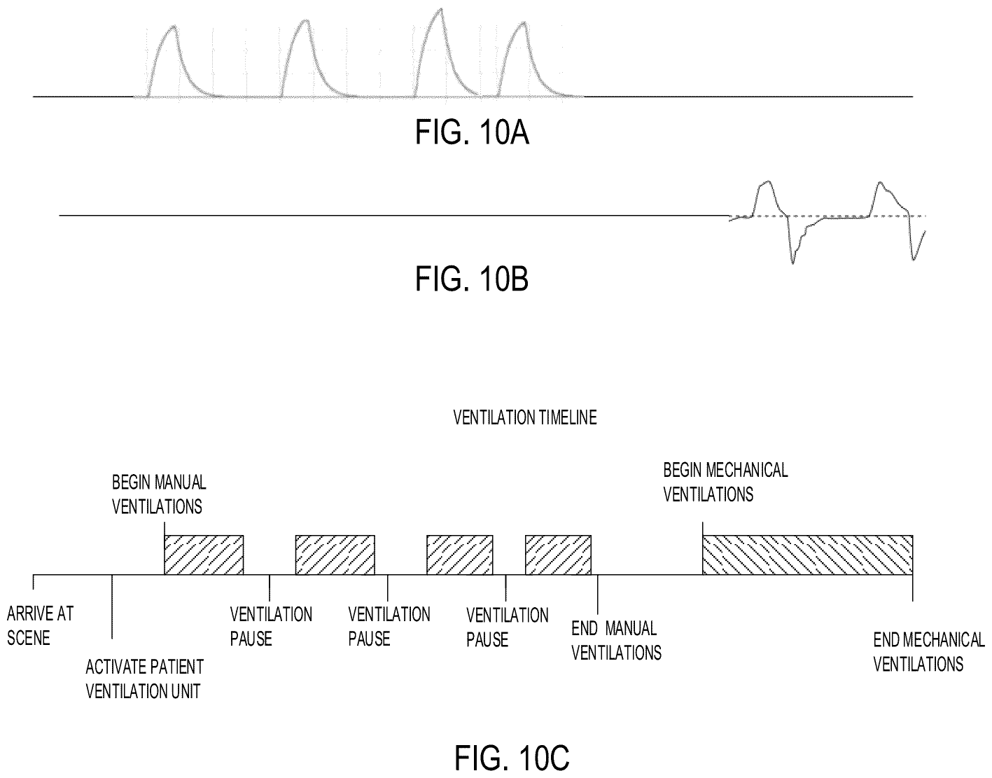

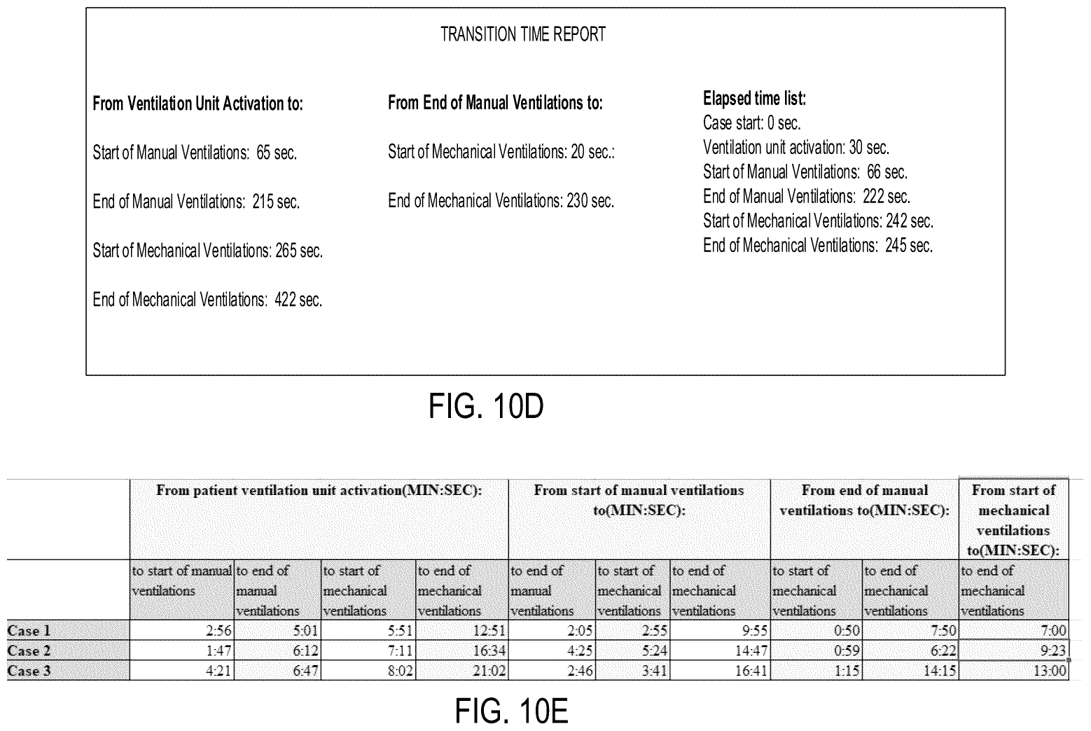

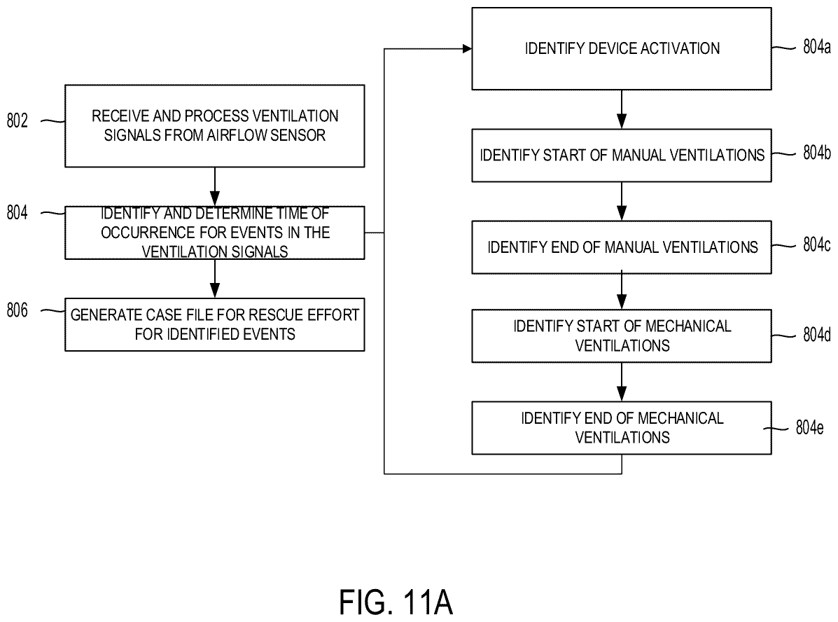

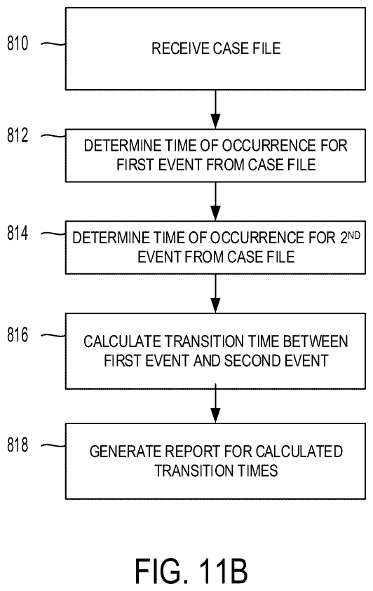

The computing device 628 of the system 600 can be configured to generate transition time reports for different types of ventilations provided to the patient 602, which are similar to the previously described reports for chest compression transition times. Specifically, the processor 630 and/or reporting engine 656 of the computing device 628 can be configured to receive a case file for a plurality of ventilation events occurring during the rescue effort and generate the report from the received case file. In particular, signals from the airflow sensor(s) 620, 622 can be processed and analyzed to identify times of occurrence for ventilation events occurring during the rescue effort. Exemplary waveforms representing signals that may be received from the airflow sensors 620, 622 are shown in FIGS. 10A and 10B. In particular, FIG. 10A shows a waveform representing signals that may be generated from the first airflow sensor 620, which measures airflow during manual ventilation of the patient 602. FIG. 10B shows a waveform representing signals that may be generated from the second airflow sensor 622, which measures airflow to the patient 602 from mechanical ventilations from the ventilator 616. The waveforms of FIGS. 10A and 10B can be evaluated or combined to produce a timeline of events occurring during the rescue effort, which is shown schematically in FIG. 10C. Specifically, FIG. 10C shows events occurring during a rescue effort including arrival at the rescue scene, a start of manual ventilations, pauses in manual ventilations, an end of manual ventilations, a start of mechanical ventilations, and an end of mechanical ventilations. Method or processing steps for identifying these ventilation events from the detected airflow signals and for generating the reports for the ventilation transition times are described in connection with FIGS. 11A and 11B.

### Transition Time Reports for Ventilations

FIGS. 10D and 10E show different types of reports that can be generated by the ventilation system 600 of the present disclosure. For example, FIG. 10D shows a Transition Time Report for patient ventilation, which could be displayed, for example, on a visual display of a technician console or another computing device 628. The report comprises a list of transition times measured from ventilation unit activation. As used herein, “ventilation unit activation” can mean activation of the airflow sensor(s) 620, 622 in the airflow path 618. Activation can occur when the airflow sensor(s) 620, 622 are connected to another medical device, such as the medical device 112 shown in FIGS. 9B and 9C. The report also comprises a list of times measured from the end of manual ventilations, as well as a list of elapsed times measured from the start of a case (i.e., arrival of the rescuers at the rescue scene). FIG. 10E shows a report in the form of a table including values for many different transition times during a rescue effort. The table can also comprise rows for other rescue events, allowing rescuers to compare performance for different rescue efforts or cases.

FIGS. 11A and 11B are flow charts showing processes or computer implemented methods performed by medical devices 112 at the rescue scene and/or by remote computing devices 628 for generating the case file of ventilation events performed for the patient 602 during the rescue effort and for generating the report. The methods of FIGS. 11A and 11B can be performed by devices, such as the medical device 112, at the rescue scene. For example, some or all of the steps of the methods of FIGS. 11A and 11B can be performed by computer processors of portable medical devices or portable computing devices at the rescue scene. In other examples, some or all of the steps of the methods of FIGS. 11A and 11B can be performed by remote computing devices 628, such as computer servers or computing devices that receive the case file and/or other data from medical devices 112 at the rescue scene.

As shown in FIG. 11A, the method can comprise, at step 802, receiving and processing time-correlated signals from the airflow sensor(s) 620, 622 representative of ventilations provided to the patient 602. For example, signals representative of manual ventilations provided to the patient 602 can be received from the first airflow sensor 620 of the manual ventilation unit 614. Signals representative of mechanical ventilations provided to the patient 620 by the mechanical ventilator 616 can be received from the second airflow sensor 622. At step 804, the method further comprises identifying and determining times of occurrence for the plurality of medical events represented in the time-correlated signals from the airflow sensor(s) 620, 622.

For example, as previously described, the medical events can comprise activation of the ventilation unit (step 804a), such as connecting the airflow sensor(s) 620, 622 to a medical device 112 or computing device 628 at the rescue scene. The events can also comprise the start of manual ventilations (step 804b), the end of manual ventilations (step 804c), the start of mechanical ventilations (step 804d), or the end of mechanical ventilations (step 804e).

More specifically, identifying and determining a time of occurrence for manual ventilations can comprise generating data representative of a ventilation waveform from the received and processed time-correlated signals received from the airflow sensor(s) 620, 622; identifying portions of the ventilation waveform representative of manual ventilations provided for the patient; and determining the latest time represented by the portions of the ventilation waveform representative of the manual ventilations. For example, as previously described, signals received from the first airflow sensor 620 can be representative of manual ventilations provided to the patient and signals received from the second airflow sensor 622 can be representative of mechanical ventilations provided to the patient 602. In a similar manner, a start of mechanical ventilations may be identified by identifying portions of the ventilation waveform representative of mechanical ventilations provided for the patient 602 and determining an earliest time represented by the portions of the ventilation waveform representative of the mechanical ventilations. At step 806, the method further comprises generating the case file for the rescue effort. As in previous examples, the case file can comprise the times of occurrence for the plurality of medical events.

FIG. 11B is a flow chart showing steps of a method for generating the report for transition times between events during ventilation of the patient. The method comprises, at step 810, receiving the case file for the rescue effort. For example, the case file can be received from a medical device 112 or computing device 628 located at the rescue scene. The method can further comprise, at step 812, selecting and determining the time of occurrence for a first event of the plurality of medical events from the case file. The method can further comprise, at step 814, selecting and determining the time of occurrence for a second event of the plurality of medical events occurring after the selected first event from the case file. At step 816, the method further includes determining or calculating a transition time between the time of occurrence of the first event and the time of occurrence of the second event. At step 818, the method further comprises generating the report that provides a transition time indication representative of the determined or calculated transition time for user review.

### Systems for Monitoring and Determining Transition Times for User Switching

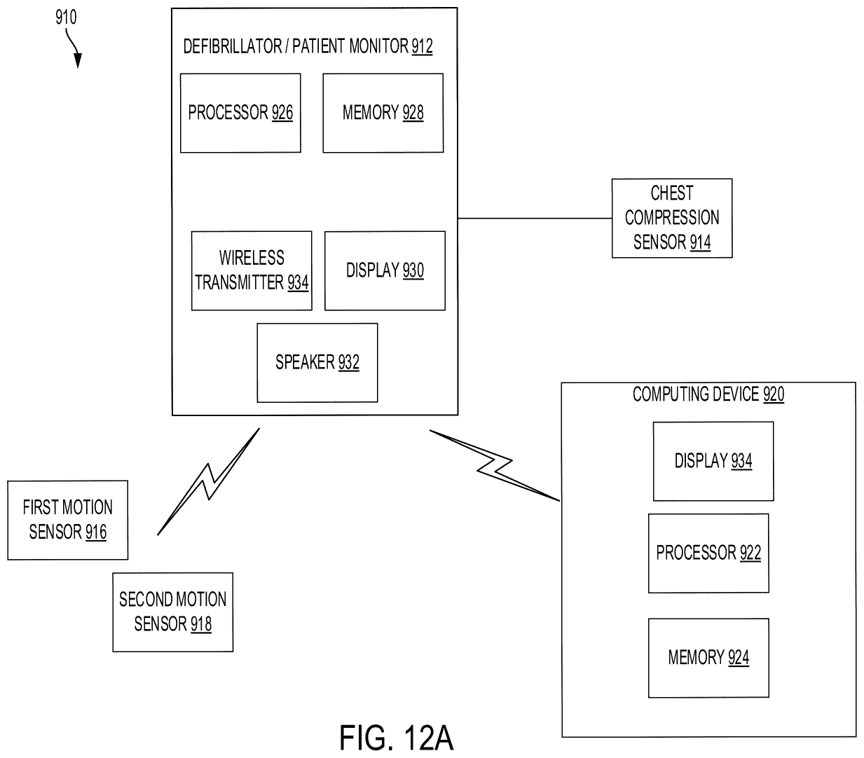

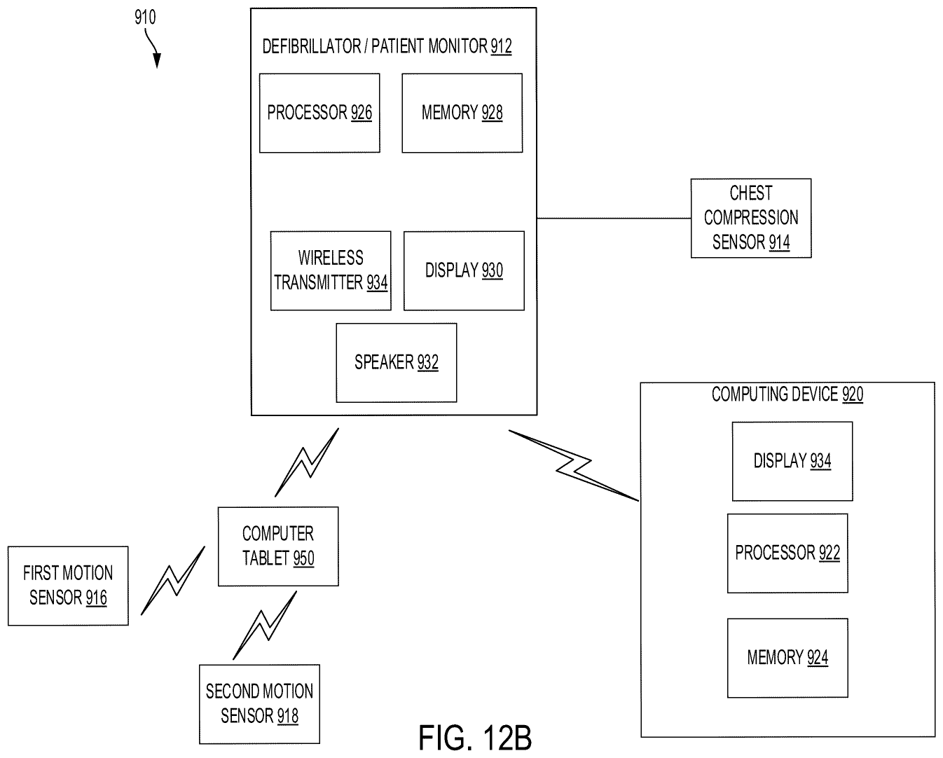

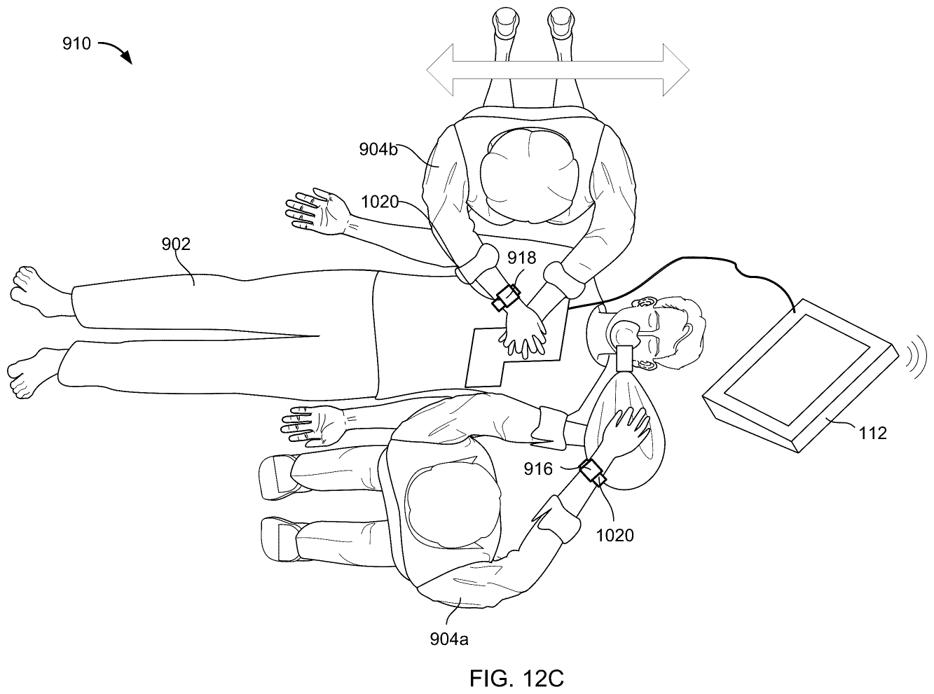

FIGS. 12A-12C illustrate components of systems for monitoring resuscitation activities performed by multiple rescuers and, in particular, for reviewing and reporting transition times between chest compressions performed by the multiple rescuers.

As shown in FIGS. 12A and 12B, the system 910 comprises a medical device 912 which comprises or is connected to a chest compression sensor 914, which can be similar or identical to the previously described chest compression sensors, configured to receive time-correlated compression signals representative of chest compressions performed for the patient. For example, the chest compression sensor 914 can be a single axis or a multi-axis accelerometer, such as an accelerometer configured to be positioned on a sternum of the patient. The accelerometer can be contained in a housing configured to be positioned on the patient's sternum between hands of a rescuer performing the chest compressions and a chest of the patient. The medical device 912 can also comprise a processor 926 and associated memory 928 for receiving and processing signals from sensors at the rescue scene and for controlling the medical device 912. The medical device 912 can also comprise output components, such as a display 930 and/or speaker 932 for providing information about device status or about the rescue effort to users. The medical device 912 can also comprise a wireless transceiver 934 for receiving data from wireless sensors at the rescue scene. In some examples, the wireless transceiver 934 can also be configured to transmit data comprising information about the medical device 912, patient, and/or rescue effort to remote computing devices or networks.

The system 910 further comprises motion sensors for detecting time-correlated movement signals representative of movement of hands or wrists of rescuers 904a, 904b (shown in FIG. 12C) at the rescue scene. For example, the system 910 can comprise a first motion sensor 916 configured to detect time-correlated movement signals representative of movement of hands or wrists of a first rescuer 904a. The system 910 can further comprise a second motion sensor 918 configured to detect time-correlated movement signals representative of movement of hands or wrists of a second rescuer 904b. In some examples, the motion sensors 916, 918 wirelessly transmit information to the medical device 912. In other examples, the motion sensors 916, 918 can transmit data to the medical device 912 that gets pre-processed via an intermediary device, such as the computer tablet 950 shown in FIG. 12B.

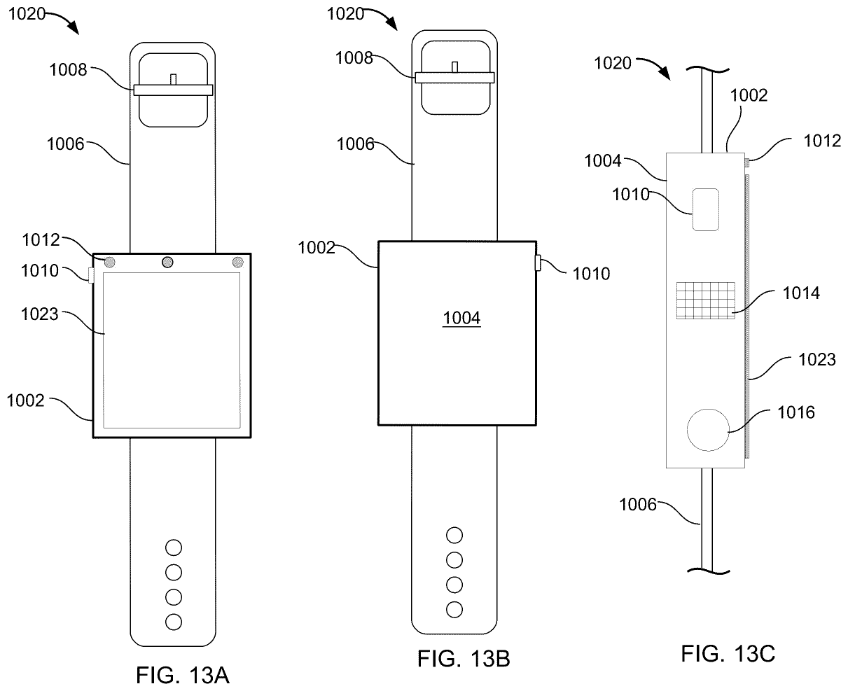

In some examples, the motion sensors 916, 918 are wearable. For example, the first motion sensor 916 and/or the second motion sensor 918 can be a wrist-worn device, such as a smart watch, configured to be worn by the rescuer 904a, 904b, as shown in FIGS. 12B and 13A-13C.

As in previous examples, the system 910 further comprises a computing device 920 having a processor 922 and computer readable memory 924 communicatively coupled with the chest compression sensor 914, the first motion sensor 916, and the second motion sensor 918. The computing device 920 can also comprise the computer readable memory 924 containing instructions for receiving and processing data from the chest compression sensor 914 and/or motion sensors 916, 918, and for generating the report from the received data. In some examples, the computing device 920 can also comprise a display 930 allowing a user, such as a technician or similar user, to review information about the rescue effort including the generated transition time report. In other examples, the computing device 920 can comprise a computer server that makes the report available to other computing devices for review by users over, for example, a computer network.

In some examples, the medical device 912 and/or computing device 920 of FIG. 12A can be configured to receive and process time-correlated compression signals from the chest compression sensor 914, as occurs in previously described examples. Unlike in previous examples, the medical device 912 and/or computing device 920 may also be configured to receive and process time-correlated movement signals either directly from the first and second motion sensors 916, 918 or through an intermediary device (which may perform pre-processing of the signals prior to transmission to the medical device 912), such as the computer tablet 950 (shown in FIG. 12B). The medical device 912 and/or computing device 920 can be configured to analyze the time-correlated compression signals and the time-correlated movement signals to identify portions of the compression signals for chest compressions by the first rescuer 904a and portions of the compression signals for chest compressions by the second rescuer 904b.

In some examples, the medical device 912 and/or computing device 920 can be configured to analyze the time-correlated compression signals and the time-correlated movement signals by determining a parameter value for multiple segments of the time-correlated compression signals. For example, parameter values can comprise compression rate, compression depth, compression hold time, variation in compression rate, variation in compression depth, variation in hold time, compression width, relaxation time, release time, compression average velocity, compression maximum velocity, or velocity minimum to maximum time (per chest compression cycle), as well as any of the features shown in FIGS. 3A and 3B, described previously. Also, values for RMS power derived from the time-correlated movement signals (e.g., accelerometer signals) for different portions of the rescue effort can be used to identify which rescuer is performing chest compression movements at particular times during the rescue effort.

More specifically, the compression signals received from the chest compression sensor can be divided into segments of equal length, such as segments of 5 seconds, 10 seconds, 30 seconds, or another convenient duration. The medical device 912 or computing device 920 can also determine a parameter value for multiple segments of the time-correlated movement signals for times corresponding to times of the segments of the time-correlated compression signals. The medical device 912 or computing device 920 can then compare the determined parameter value for the multiple segments of the time-correlated compression signals to the determined parameter values for the multiple segments of the time-correlated movement signals in order to identify segments of the compression signals and motion signals having similar or identical parameter values.

When parameter values for the compression signals match parameter values for motion signals received from the first motion sensor 916, it indicates that compressions were performed by the first rescuer 904a. When parameter values for compression signals match parameter values for motion signals detected by the second motion sensor 918, it indicates that the chest compressions were performed by the second rescuer 904b. Accordingly, the computing device 920 can be configured to identify particular segment(s) of the chest compression signal as first rescuer segment(s) when the determined parameter value for the particular segment(s) of the time-correlated compression signals are within a predetermined amount of the parameter value (e.g., a value such as displacement, velocity, or acceleration detected by the motion sensor) for the time-correlated movement signal for the first motion sensor 916. In a similar manner, the computing device 920 can be configured to identify particular segment(s) as second rescuer segment(s) when the parameter value for the particular segment(s) of the time-correlated compression signals is within a predetermined amount of the parameter value for the time-correlated movement signal for the second motion sensor 918.

Based on the analysis, the computing device 920 can be configured to identify and determine a time of occurrence for a first event occurring during identified portions of the compression signals for chest compressions by the first rescuer 904a, and to identify and determine a time of occurrence for a second event occurring during identified portions of the compression signals for chest compressions by the second rescuer 904b. The computing device 920 is further configured to determine a transition time between the time of occurrence of the first event and the time of occurrence of the second event, and cause a transition time indication representative of the determined transition time to be displayed, for example, on a visual display 934 of the computing device 920 or on a user's computer via, for example, a website.

In some examples, the transition times determined or calculated by the computing device 920 can be between at least one of: (i) a start of chest compressions by the first rescuer and an end of chest compressions by the first rescuer, (ii) the start of chest compressions by the first rescuer and a start of chest compressions by the second rescuer, (iii) the start of chest compressions by the first rescuer and an end of chest compressions by the second rescuer, (iv) the end of chest compressions by the first rescuer and the start of chest compressions by the second rescuer, (v) the end of chest compressions by the first rescuer and the end of chest compressions by the second rescuer, (vi) the start of chest compressions by the second rescuer and the end of the chest compressions by the second rescuer, and/or (vii) the end of chest compressions by the second rescuer and a restart of chest compressions by the first rescuer.

In some examples, the motion sensors 916, 918 can be components of wrist-worn devices 1020, such as smart watches, as shown in FIGS. 12B and 13A-13C. Exemplary wrist-worn devices, which can be used for collecting information about resuscitation activities performed by rescuers at a rescue scene and/or for providing feedback about resuscitation activities performed by rescuers, which can be used with the systems 910 of the present disclosure and described, for example, in U.S. Pat. No. 11,202,579, entitled “Wrist-worn device for coordinating patient care,” which is incorporated herein by reference in its entirety.

With reference to FIGS. 13A-13C, an exemplary wrist-worn device 1020 is illustrated. The exemplary wrist-worn device 1020 comprises electronic circuitry for storing and processing received data. The circuitry is enclosed in a case or housing 1002. The housing 1002 can be formed from a suitable protective material, such as a hard plastic or metal (e.g., brushed aluminum). The housing 1002 can be a suitable shape and size to rest against the wrist of a user. For example, a bottom surface 1004 of the housing 1002 can be flat or curved to rest against the user's wrist. The device 1020 can comprise a wrist strap 1006, formed from a flexible material, such as rubberized plastic, elastic, leather, or fabric. In some embodiments, the strap 1006 is made of a flameproof material. The strap 1006 can comprise a clasp or buckle 1008 for holding the device 1020 against the user's wrist, or a magnetic clasp or slap bracelet. The strap 1006 itself may comprise one or more sensors for measuring physiological parameters/status of the user, similar to those discussed above with respect to the smartwatch.

In some examples, the device 1020 comprises at least one visual display 1023. The display 1023 can be a touch screen display, allowing the user to control operation of, enter information, and interact with the device 1020 by the display 1023. In some examples, the display 1023 can be substantially flexible and/or curved so that the housing 1002 more easily rests against the wearer's wrist. In addition, a curved display has an increased surface area compared to a flat display, meaning that a greater amount of information can be shown on the curved display. For example, the display 1023 can be made of Indium gallium zinc oxide (IGZO), a semiconducting material. IGZO thin-film transistors (TFT) can be used in the TFT backplane of flat-panel displays (FPDs).

In some examples, the wrist-worn device 1020 can comprise an input mechanism(s), such as physical buttons 1010, for allowing additional interaction activities with the device 1020. Other types of input mechanisms that can be integrated with a wrist-worn device 1020 can comprise rotatable dials, keyboards, number pads, and the like. In some examples, the button 1010 can be a “Home Screen” or “Main Menu” button that when pressed returns the visual display 1023 to a home screen, from which various features of the device 1020 can be actuated or controlled. Other buttons 1010 can comprise an acknowledgement button or “OK” button for acknowledging or confirming notifications displayed on the device 1020. Other buttons 1010 can be used to toggle or otherwise navigate through various user interface screens or notifications provided by the device 1020. The device 1020 may further provide a component that allows for the user to provide input via a rotatable motion. Such a component may be provided as a rotatable dial as discussed above, or may employ rotary encoders that sense movement (e.g., circular/rotational motion, fingertip encircling the encoders) around the component, for example, to scroll through a series of options for viewing and/or selection (e.g., treatments, DTA Marker inputs, visual displays, physiological parameters, etc.). An example of an input component that senses rotary motion, as known to those skilled in the art, includes the digital crown feature provided with the APPLE Watch.

The device 1020 can further comprise a visual indicator 1012 located on the device housing 1002 for conveying different types of information, alerts, or notifications to the user or other personnel. For example, the visual indicators 1012 can be colored lights (e.g., LEDs) that flash to signal that the device 1020 has received an alert or notification.

The device 1020 can also comprise audio output components, such as speakers 1014, for emitting audible alerts, and audio input components, such as a microphone port 1016, for recording speech and/or environment noise. The device 1020 can comprise at least one other port or opening that provides access to other types of sensors. For example, motion, optical, and physiological sensors can be enclosed within the housing 1002.

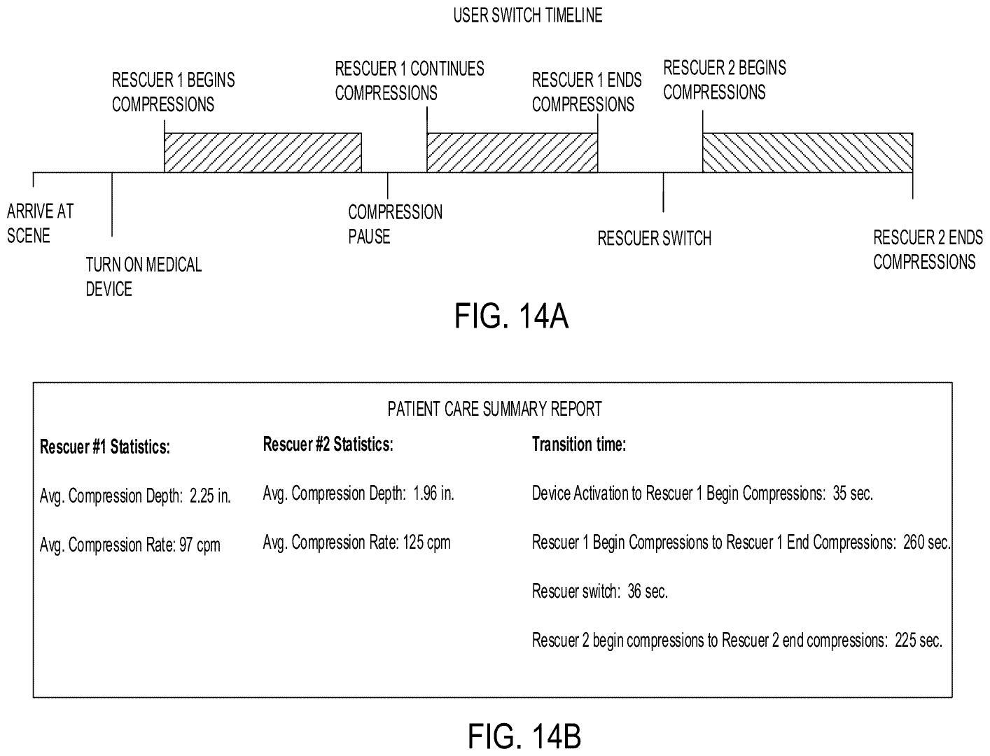

FIG. 14A shows a timeline of events, which can occur during a rescue effort, and which can be detected by processing the chest compression signals from the chest compression sensor 914 and the motion signals from the motion sensors 916, 918 shown in FIGS. 12A and 12B. For example, as shown in the timeline, events occurring during the rescue effort can include arrival at the rescue scene and turning on or activation of medical device(s) 912 at the rescue scene. Events can also include, for example, a start of chest compressions by the first rescuer 904a, pauses in chest compressions, an end of chest compressions by the first rescuer 904a, a start of chest compressions by the second rescuer 904b, and an end of chest compressions by the second rescuer 904b. As previously described, the times of occurrence for the events shown in the timeline can be determined by comparing the chest compression signals from the compression sensor 914 with the motion signals from the motion sensors 916, 918 to determine which compressions were performed by the first rescuer 904a and which compressions were performed by the second rescuer 904b.

FIG. 14B shows an exemplary Patient Care Summary or Transition Time Report that can be generated showing statistics for rescuers 904a, 904b during a rescue effort. For example, displayed statistics can include numerical values for average chest compression depth for chest compressions performed by the first rescuer 904a and the second rescuer 904b. The Care Summary can also include a list of transition times determined or calculated by the system 910, as previously described. For example, the Care Summary can include transition times for a time from device activation until the first rescuer begins chest compressions. The Care Summary can also include a rescuer switch transition time representing the time from when the first rescuer 904a ends compressions until the second rescuer 904b begins compressions.

### Systems for Monitoring and Determining Transition Times for Heart Attack Events

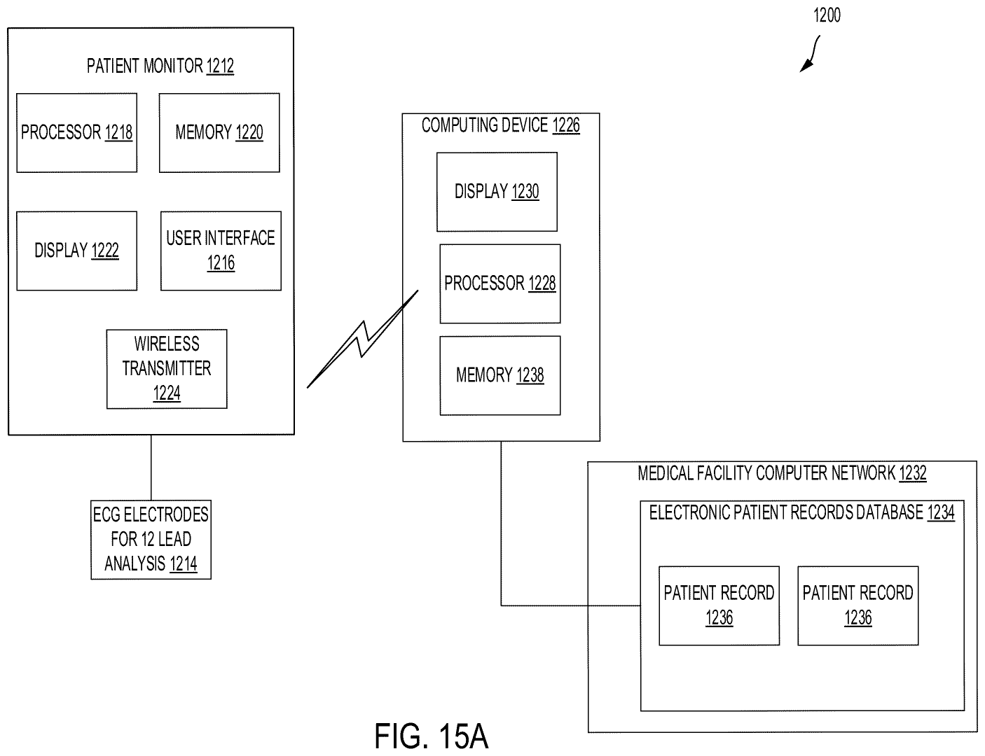

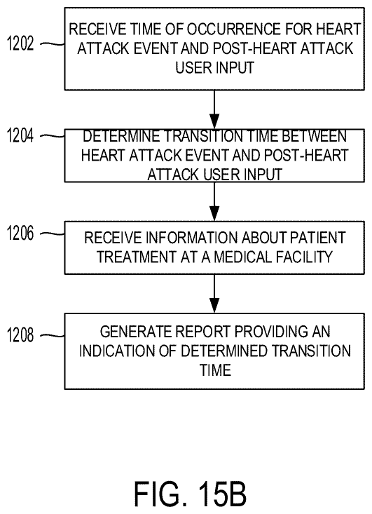

FIG. 15A illustrates components of a system 1200 for monitoring a transition time between detection of a heart attack event and a post-heart attack event user input. As used herein, the “heart attack event” can be an ST-elevation myocardial infarction (STEMI). The “post-heart attack event user input” can comprise an instruction entered by a user to transmit a heart attack notification to a remote computing network or device. Alternatively or in addition, the post-heart attack event user input can be a user input confirming that a patient treatment action, such as administration of a drug (e.g., an epinephrine injection), has been performed for the patient.

As shown in FIG. 15A, the system 1200 comprises a patient monitor 1212 comprising multiple electrocardiogram (ECG) electrodes 1214, which can be configured to be attached to a cardiothoracic region of a patient for receiving electrocardiogram signals. In particular, the multiple ECG electrodes 1214 can be configured to obtain ECG signals sufficient to create a 12 lead ECG for the patient. In some examples, the patient monitor 1212 can be solely a monitoring device configured to monitor patient signals, such as signals representative of patient vital signs, and to provide visual output and reports about detected patient signals. The patent monitor 1212 can also be configured to emit notifications or alarms when detected patient signals are abnormal or unexpected. In some examples, the patient monitor 1212 can also comprise and/or be associated with or connected to a therapeutic medical device, such as a defibrillator, ventilator, chest compressor, or other therapeutic medical devices, which can be used during a rescue effort, as are known in the art.

The patient monitor 1212 can further comprise a user interface 1216 for providing information about treatment for the patient. The user interface 1216 can be implemented on a visual display 1222 and can include, for example, portions of the display screen that provide patient information and feedback about rescue activities performed for a patient by a rescuer. The user interface 1216 can also include buttons or other data-entry icons allowing users, such as rescuers, to enter information about the patient and/or about the rescue effort. In some examples, the display 1222 can be a touch screen allowing the user to interact with the user interface by pressing virtual buttons provided at different locations on the touch screen. In other examples, the patient monitor 1212 can comprise physical buttons, switches, tracking pads, or similar input components, allowing the user, such as the rescuers, to interact with the user interface and to enter information about the patient and/or rescue effort.

The patient monitor 1212 further comprises a processor 1218 and associated memory 1220 in communication with the ECG electrodes 1214 and with the user interface 1216. The processor 1218 can be configured to receive and process the ECG signals, detect and record a time of occurrence of a heart attack event based on analysis of the ECG signals, and cause a visual and/or audio notification about the heart attack event to be provided, indicating detection of the heart attack event. For example, the processor 1218 can cause a visual indication of the detected heart attack event to be displayed on the visual display 1222 of the patient monitor 1212. The processor 1218 can also be configured to receive and record a time of occurrence for a post-heart attack event user input entered via the user interface.

In some examples, the patient monitor 1212 further comprises a wireless data transceiver 1224. The processor 1218 of the patient monitor 1212 can be further configured to cause the wireless data transceiver 1224 to transmit the time of occurrence of the detected heart attack event and the time of occurrence of the instruction to transmit the heart attack notification to the remote computing network or device via the wireless data transceiver 1224.

As in previous examples, the system 1200 further comprises a computing device 1226, which can comprise a processor 1228 communicatively coupled with the patient monitor 1212. The computing device 1228 and associated computer readable memory 1238 can be configured to receive the recorded time of occurrence for detection of the heart attack event and the recorded time of occurrence for the post-heart attack event user input, determine a transition time between the time of occurrence of the heart attack event and the time of occurrence of the post-heart attack event user input, and generate a report that provides an indication representative of the determined transition time. As in previous examples, the report can be displayed on, for example, a display 1230 of the computing device 1226. Alternatively, as in previous examples, the computing device 1226 can be a computer server that makes the generated report available to users over, for example, on a website or computer network location.

In some examples, the computing device 1226 can also be in communication with and/or configured to receive information from a medical facility computer network 1232, such as a network comprising an electronic patient record database 1234 comprising patient records 1236 for one or more patients. For example, the computing device 1226 can be configured to receive information about treatment of the patient by a medical facility after the rescue event. The received information about the treatment of the patient can include, for example, drug administration time(s) for drugs administered to the patient at the medical facility and/or a time when a stent was implanted or catheter balloon treatment was provided for the patient (often referred to as a patient “stent time” or “balloon time”). Based on the received information about patient treatment at the medical facility, the computing device 1226 can be configured to determine a transition time between events occurring during the rescue effort and events occurring at the medical facility. For example, the computing device 1226 can be configured to determine a transition time between detection of the heart attack event (e.g., detection of STEMI) and administration of a drug at the medical facility or a time when the stent was implanted or catheter balloon treatment was provided for the patient. The determined or calculated transition times between events occurring during the rescue effort and events occurring at the medical facility can be included in the report generated by the computing device 1226.

FIG. 15B is a flow chart illustrating a method or process performed by the system 1200 of FIG. 15A for generating the transition time report for the heart attack event and the post-heart attack user input. Specifically, as shown in FIG. 15B, at step 1202, the method comprises receiving a recorded time of occurrence for detection of the heart attack event and a recorded time of occurrence for a post-heart attack event user input, such as the time that an instruction to transmit a heart attack notification to a remote computing network or device of a medical facility or the time that a drug is administered to the patient by rescuers during the rescue effort. At step 1204, the method further comprises determining a transition time between the time of occurrence of the heart attack event and the time of occurrence of the post-heart attack event user input. At step 1206, the method further optionally comprises receiving information about treatment of the patient by a medical facility after the rescue event, such as information about medications delivered to the patient at the medical facility and/or a stent time or balloon time (e.g., a time that a stent was implanted or catheter balloon treatment was provided for the patient). At step 1208, the method further comprises generating a report that provides an indication representative of the determined transition time between the heart attack event and the post-heart attack user input. The report can also include transition times between the heart attack event and actions performed at the medical facility.

### System and Methods for Generating a Report Including Transition Time Trends

With reference again to FIGS. 1A and 1B, in some examples, the systems 100 and methods of the present disclosure can also be used for collecting data from multiple rescue efforts in order to provide information about how a particular team of rescuers works together over time. For example, a system 100 for reporting transition time trends in patient care data can comprise a computing device 116 including features of previously described computing devices, such as the computing device 116 shown in FIG. 1A or 1B. In some examples, the computing device 116 of the system 100 can be in communication with and/or capable of receiving data from multiple medical devices 112 and/or data about multiple rescue efforts. For example, the computing device 116 can be configured to receive and process a plurality of case files generated by one or more medical devices 112 during different rescue efforts. Each of the plurality of case files can comprise times of occurrence for a plurality of events related to performance of a resuscitation activity, such as chest compressions or ventilations, performed for a patient during a rescue effort.

In some examples, the computing device 116 can be configured, for each received case file, to select and determine a time of occurrence for a first event of the plurality of events of the case file, and select and determine a time of occurrence for a second event of the plurality of events in the case file, which occurs after the selected first event. The computing device 116 can also be configured to determine a transition time between the first event and the second event for each of the plurality of received case files. Once the transition time is determined or calculated, the computing device 116 can be configured to generate a report that provides a transition time indication representative of the determined or calculated transition time for user review.

The resuscitation activity documented in the received case files can be any of the previously described resuscitation activities described in connection with other systems and methods of the present disclosure. For example, the resuscitation activity can comprise manual chest compressions, automated chest compressions, manual ventilations, and/or automated ventilations.

The system 100 can be configured to generate transition times between events related to any of these resuscitation activities performed by one or more rescuers during the multiple rescue efforts. For example, the transition time can be a transition time between at least one of: (i) a start of the manual chest compressions and an end of the manual chest compressions, (ii) the start of the manual chest compressions and a start of the automated chest compressions, (iii) the start of the manual chest compressions and an end of the automated chest compressions, (iv) the end of manual chest compressions and the start of automated chest compressions, (vi) the end of manual chest compressions and the end of automated chest compressions, or (vii) the start of automated chest compressions and the end of automated chest compressions. The transition time can also be a transition time between at least one of (i) a start of the manual ventilations and an end of the manual ventilations, (ii) the start of the manual ventilations and a start of the mechanical ventilations, (iii) the start of the manual ventilations and an end of the mechanical ventilations, (iv) the end of the manual ventilations and the start of the mechanical ventilations, (v) the end of the manual ventilations and the end of the mechanical ventilations, or (vi) the start of the mechanical ventilations and the end of the mechanical ventilations.

In some examples, the computing device 116 can be configured to generate a report or summary that provides overall transition time values for multiple rescue efforts. For example, the computing device 116 can be configured to generate a report that comprises an average transition time value for multiple case files and/or generated from data collected over multiple rescue efforts. For example, the generated report could include an average transition time (e.g., a transition time between an end of manual compressions and a start of automated compressions) for multiple rescue efforts. In some examples, the multiple rescue efforts can be different rescue efforts with the same rescuers or team of rescuers collected over a period of time, such as a week or month. In other example, the multiple rescue efforts can be for rescues performed by different rescuers giving, for example, evidence of average care quality provided by all rescuers of a particular emergency care team.

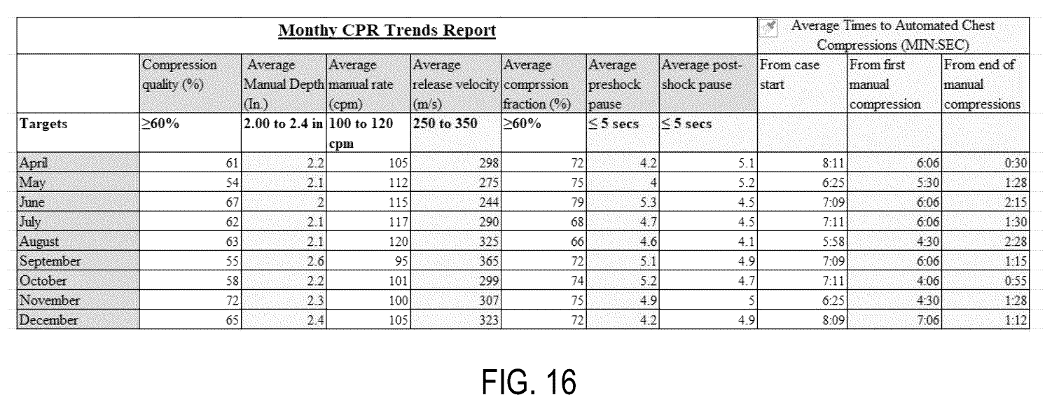

FIG. 16 shows an exemplary Monthly CPR Trends Report including the transition trend data determined by combining data collected during multiple rescue efforts and/or performed by multiple rescuers. As shown in FIG. 16, the table includes rows for different months (e.g., from April until December). The table also includes columns for information about resuscitation quality statistics or parameters, such as compression quality, average manual compression depth, average manual compression rate, average release velocity, average compression fraction, average pre-shock pause, and average post-shock pause. The table also includes transition time information, specifically the average times to automated chest compressions from (i) case start, (ii) first manual compression, and (iii) end of manual compressions. As previously described, the per-month average transition time values in the table can be based on all rescue efforts performed by a particular rescuer or rescue team over the month.

### Real-Time Feedback Systems

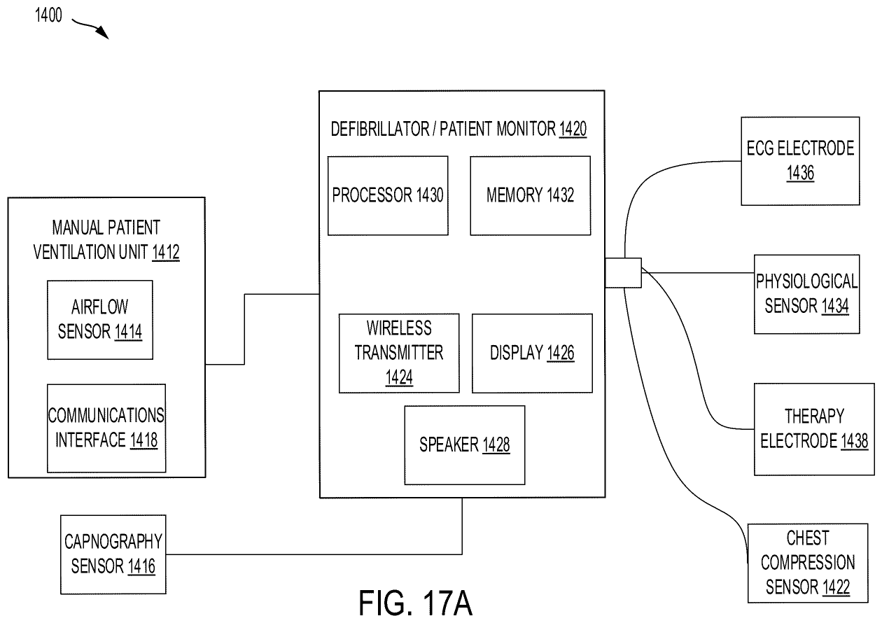

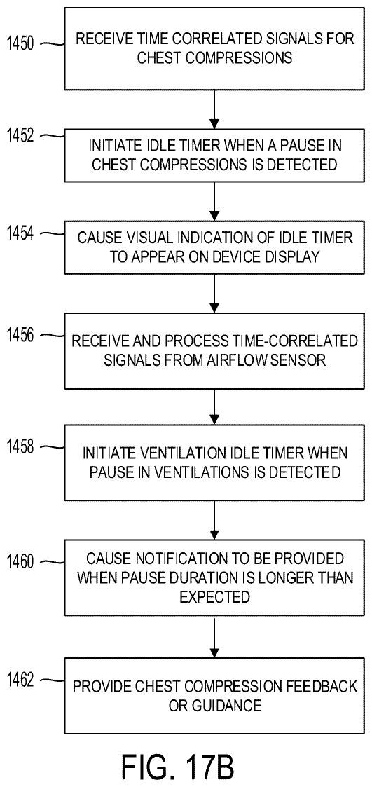

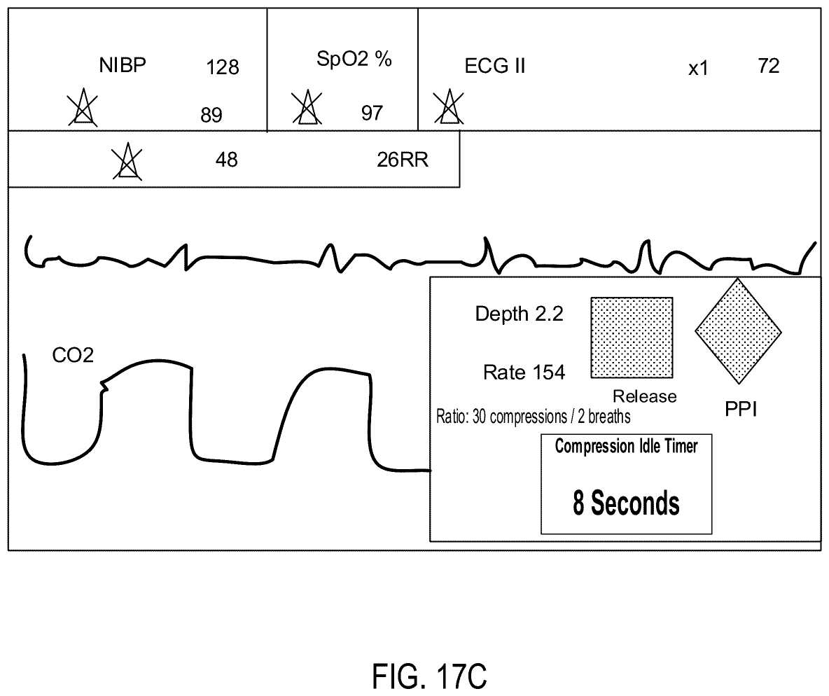

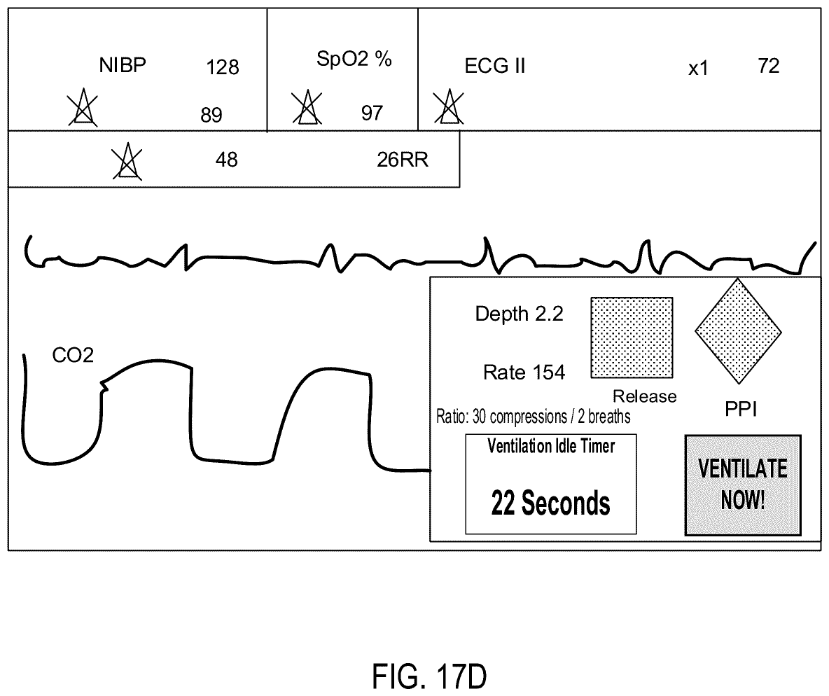

FIG. 17A illustrates a system 1400 for providing real time or substantially real time feedback to rescuers about patient care, which can include feedback about transition times and/or about an elapsed time after performing a patient treatment action. The feedback can be provided, for example, on a display of a medical device, such as a defibrillator or ventilator, and/or on a display of a portable computing device at a rescue scene. As used herein, feedback can refer to prompts, notifications, displays of chest compression information, and/or instructions, including haptic feedback, audible feedback, and/or visual feedback, which assists or guides a rescuer in performance of the chest compressions for a patient in accordance with a selected protocol, criteria, or parameters. Chest compression parameters can include, for example, compression force, compression rate (in compressions per minute), measured compression depth, and/or a decompression velocity (e.g., a release velocity). Chest compression parameters that can be measured or derived from information detected by chest compression sensor(s) can also comprise compression hold time, downstroke acceleration, downstroke velocity, lift displacement, lift force, upstroke acceleration, and upstroke velocity.

For example, the information from the chest compression sensor(s) may be used to determine, calculate, and/or estimate present values for the chest compression evaluation criteria or parameters. In that case, the feedback may provide an indication of the present values for the chest compression parameters. The feedback can also comprise information about target values for chest compression parameters and/or recommended changes to measured chest compression parameters or values relative to the target values. For example, the feedback can comprise indications to increase or decrease compression depth depending on whether the measured compression depth falls within a desired target range for compression depth, instructions to compress at a faster or slower rate depending on whether the measured compression rate falls within a desired target range for compression rate, and/or indications to quickly and completely release the chest of the patient after each compression depending on whether the measured release velocity falls within a desired target range for release velocity. In general, feedback may be corrective feedback (i.e., feedback configured to cause the rescuer to change an aspect of the resuscitative care) and/or may be reported measurements (i.e., feedback that indicates a value or status of an aspect of the resuscitative care without a suggested change).

With continued reference to FIG. 17A, the system 1400 comprises a manual patient ventilation monitoring unit 1412, which can be similar or identical to the manual ventilation unit shown in FIGS. 9A-9C. The manual patient ventilation unit 1412 can comprise an airflow path configured to be in fluid communication with an airway of the patient for providing ventilations to the patient. For example, a ventilation bag (shown in FIG. 9B) can be used for providing manual ventilations to the patient through the airflow path. The airflow path can comprise an airflow sensor 1414 positioned to sense time-correlated signals representative of airflow in the patient's airway, as well as a communication interface 1418 for transmitting signals detected by the airflow sensor 1414 to another device at the rescue scene, such as to a patient monitor or defibrillator. The system 1400 can also comprise a capnography sensor 1416 for detecting, for example, end tidal carbon dioxide values for the patient.

The system 1400 can further comprise a medical device 1420, such as a patient monitor or defibrillator, which can include features of previously described medical devices. For example, the medical device 1420 can comprise a chest compression sensor 1422, which is similar or identical to previously described chest compression sensors, configured to receive time-correlated signals representative of chest compressions performed for the patient. The medical device 1420 (e.g., the defibrillator or patient monitor) can further comprise a wireless transmitter 1424 for transmitting information detected by the airflow sensor 1414 and/or chest compression sensor 1422 to remote computing networks or devices. As in previous examples, the medical device 1420 can also comprise physiological sensors 1434, ECG electrodes 1436, and therapy electrodes 1438 for monitoring patient condition and for providing cardiac therapy to the patient.

The system 1400 further comprises a visual display 1426 for providing information about the chest compressions and ventilations performed for the patient. For example, as shown in FIG. 17A, the visual display 1426 can be a display of the medical device 1420. In other examples, the visual display 1426 can be a display of a separate portable electronic device, which can be used at a rescue scene, such as a smart phone, computer tablet, or a similar device. The medical device 1420 can also comprise speakers 1428 for providing audio output about chest compressions or ventilations performed for the patient.

In some examples, the system 1400 further comprises a computer processor 1430 and associated computer readable memory 1432 in communication with the chest compression sensor 1422, airflow sensor 1414, and the visual display 1426. As shown in FIG. 17A, the processor 1430 can be a computer processor of the medical device 1420, such as the defibrillator or patient monitor. In other examples, the processor 1430 can be a processor of another electronic device at the rescue scene or a processor of another computing device or computer server remote from the rescue scene, which can be connected to and/or can receive data from the medical device 1420, such as the defibrillator or patient monitor, over a computer network.

The processor 1420 is configured to monitor resuscitation activities performed for the patient by rescuers and to provide feedback to a user about the performed resuscitation activities. FIG. 17B is a flow chart showing steps of a computer implemented method or process, which can be performed by the processor 1430 for generating and providing the user feedback. This feedback may be particularly relevant for CPR protocols that involve transitions between chest compressions and ventilations, such as 30:2 compressions:ventilations ratio. In such a situation, for a 30:2 protocol, once 30 chest compressions are provided, then the system 1400 may prompt the user to provide 2 ventilations. If there is a significant pause in the transition between compressions and ventilations, then the system 1400 may provide feedback for the appropriate caregiver to provide ventilations. Once the correct number of ventilations have been administered such that CPR should transition back to compressions, then feedback may then be provided for the appropriate caregiver to provide compressions. As shown at step 1450, the method comprises receiving and processing time-correlated signals from the chest compression sensor 1422 to identify times of occurrence for the chest compressions. At step 1452, the method further comprises initiating an idle timer when a premature pause in chest compressions is detected in the processed time-correlated signals. For example, pauses in chest compressions can be identified by monitoring an elapsed time (e.g., using the accelerometer signal from the compression sensor) since a most-recent chest compression was performed for the patient and determining that there is a pause in chest compressions when the elapsed time exceeds a time permitted by a selected CPR protocol for the patient by at least a predetermined amount. For example, a CPR protocol may require that chest compressions should be paused by no more than 1 second, 5 seconds, 10 seconds, or another selected duration. If the processor 1430 determines that compressions are paused by longer than the selected duration, the processor 1430 can be configured to initiate the idle timer. At step 1454, the method further comprises causing a visual indication of the idle timer to be displayed on a visual display, such as the display 1426 of the medical device 1420. However, when the appropriate number of compressions has been provided (e.g., 30 compressions in a 30:2 protocol), then the method may allow for feedback to be provided to the caregiver prompting the caregiver(s) to pause chest compressions and for ventilations to begin. It can then be determined whether compressions have indeed paused at the appropriate time using processed signals from the chest compression sensor, and also whether ventilations have been administered using processed signals from the airflow sensor.

At step 1456, the method further comprises receiving and processing time-correlated signals from the airflow sensor 1414 about ventilations provided for the patient to verify that ventilations are indeed being provided to the patient. At step 1458, the method further comprises initiating a ventilation idle timer when an undesirable pause in ventilations is detected. For example, identifying a pause in ventilations can comprise monitoring an elapsed time since a most-recent ventilation was provided to the patient and determining that there is a pause in ventilations when the elapsed time exceeds a time permitted by the CPR protocol, such as a protocol of 30 compressions followed by 2 ventilations. For example, a CPR protocol may require that ventilations should be provided to a patient every 10 seconds, every 20 seconds, or every 30 seconds. If ventilations are not provided in the expected time, the processor 1430 can be configured to initiate the ventilation idle timer. At step 1460, the method further comprises causing a notification or alarm to be provided on the visual display when the pause in ventilations is longer than a predetermined acceptable ventilation interval. A visual indication for the ventilation idle timer can also be shown on the visual display proximate to the notification or alarm. At step 1462, optionally, the method further comprises analyzing the received and processed signals for the chest compressions and providing feedback on, for example, the display 1426 and/or speakers 1428 of the medical device 1420, for guiding the caregiver in performing chest compressions according to the CPR protocol.

FIGS. 17C and 17D illustrate defibrillator display screens that can be shown on the display of the defibrillator or patient monitor of the system 1400 of FIG. 17A. The display screen can include physiological information for the patient, such as an ECG trace or an end-tidal CO2 waveform. The display screen can also comprise feedback about chest compressions performed for the patient including numerical values for depth and rate, as well as visual indicators providing feedback about a quality of compression release and/or perfusion accomplished by the chest compressions provided for the patient. As shown in FIG. 17C, the display screen also includes the compression timer, showing a time since a last chest compression was detected. By contrast, FIG. 17D discloses a defibrillator display screen including a ventilation idle timer and a ventilation notification instructing the rescuer to “Ventilate Now!” meaning that the rescuer should immediately provide a manual or mechanical ventilation to the patient. As previously described, the ventilation idle timer and/or notification appears on the display screen when ventilations have not been detected in the airflow sensor 1414 signal for longer than a predetermined acceptable period of time. Once a ventilation is detected in the signal(s) from the airflow sensor, the ventilation notification can be removed and/or can be replaced with another feedback icon or indication, such as with the Compression Idle Timer shown in FIG. 17C. In some examples, idle time values measured by the compression idle timer and/or the ventilation idle timer can be recorded by the medical device 1420. The recorded values from the idle timer(s) can be added to a case file for a rescue effort and provided to a computing device in communication with the medical device 1420 for inclusion in any of the previously described transition time and CPR summary reports generated by the computing device.

### Computing Devices and Computer Systems

As will be appreciated by those skilled in the art, the processes and methods described herein can be implemented in digital electronic circuitry, or in computer hardware, firmware, and/or software. Further, features of the apparatuses described herein, including automated chest compressors, feedback units, medical devices, and chest compression feedback devices, can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can also be implemented advantageously in one or more computer programs that are executable on a programmable system including at least one programmable processor coupled to receive data and instructions from, and to transmit data and instructions to, a data storage system, at least one input device, and at least one output device.

Some of the configurations described herein are described as a process depicted as a flow diagram or block diagram. Although each flow diagram or block diagram may describe the operations as a sequential process, many of the operations can be performed in parallel or concurrently. In addition, the order of the operations may be rearranged. A process may have additional stages or functions not included in the figures. Furthermore, examples of the methods may be implemented by hardware, software, firmware, middleware, microcode, hardware description languages, or any combination thereof. When implemented in software, firmware, middleware, or microcode, the program code or code segments to perform the tasks may be stored in a non-transitory processor-readable medium such as a storage medium. Processors may perform the described tasks.

In the figures, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the scope of the disclosure.

The computer memory described herein can refer to internal computer memory, such as dynamic computer memory, as well as to computer storage devices and systems, as are known in the art. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. Common forms of physical and/or tangible processor-readable may further comprise a floppy disk, a flexible disk, hard disk, magnetic tape, or any other magnetic medium, a CD-ROM, any other optical medium, punch cards, paper tape, any other physical medium with patterns of holes, a RAM, a PROM, EPROM, a FLASH-EPROM, any other memory chip or cartridge, a carrier wave as described hereinafter, or any other medium from which a computer can read instructions and/or code.

The controllers and processors disclosed herein may be part of a computer system that includes a back-end component, such as a data server, or that includes a middleware component, such as an application server or an Internet server, or that includes a front-end component, such as a client computer having a graphical user interface or an Internet browser, or any combination of them. The components of the systems described herein can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet. The computer system can include clients and servers. A client and server are generally remote from each other and typically interact through a network, such as the described one. The relationship of client and server arises by virtue of computer programs running on the respective computers and having a client-server relationship to each other.

Having described several example configurations, various modifications, alternative constructions, and equivalents may be used without departing from the disclosure. For example, the above elements may be components of a larger system, wherein other rules may take precedence over or otherwise modify the application of aspects of the present disclosure. Also, a number of operations may be undertaken before, during, or after the above elements are considered. Also, technology evolves and, thus, many of the elements are examples and do not bound the scope of the disclosure or claims. Accordingly, the above description does not bound the scope of the claims.

1. A system for monitoring and/or reviewing transitions between types of medical treatment events provided for a patient during a rescue effort, the system comprising:
at least one medical device comprising at least one chest compression sensor configured to receive time-correlated signals representative of chest compressions performed for the patient, wherein the at least one medical device is configured to generate a case file for the rescue effort comprising times of occurrence for a plurality of medical events; and
at least one computing device having at least one processor communicatively coupled with the at least one medical device, the at least one computing device configured to:
receive the case file for the rescue effort from the at least one medical device,
select and determine the time of occurrence for at least one first event of the plurality of medical events from the case file,
select and determine the time of occurrence for at least one second event of the plurality of medical events from the case file occurring after the selected at least one first event,
determine a transition time between the time of occurrence of the at least one first event and the time of occurrence of the at least one second event, and
generate a report that provides a transition time indication representative of the determined transition time for user review.

2. The system of claim 1, wherein the at least one transition time is between at least one of:
(i) turning on the at least one medical device and a start of manual chest compressions,
(ii) turning on the at least one medical device and an end of manual chest compressions,
(iii) turning on the at least medical device and a start of automated chest compressions,
(iv) turning on the at least one medical device and an end of automated chest compressions,
(v) the start of the manual chest compressions and the end of the manual chest compressions,
(vi) the start of the manual chest compressions and the start of the automated chest compressions,
(vii) the start of the manual chest compressions and the end of the automated chest compressions,
(viii) the end of manual chest compressions and the start of automated chest compressions,
(ix) the end of manual chest compressions and the end of automated chest compressions, or
(x) the start of automated chest compressions and the end of automated chest compressions.

3. The system of claim 1, wherein the at least one medical device comprises a patient monitor comprising at least one patient physiological sensor configured to detect signals representative of at least one patient vital sign.

4. The system of claim 3, wherein the at least one patient vital sign comprises at least one of patient blood oxygen level, patient blood pressure, patient oxygen saturation (SPO₂), patient end-tidal CO₂, or patient heart rate.

5. The system of claim 3, wherein the at least one patient physiological sensor comprises at least one electrocardiogram (ECG) sensor.

6. The system of claim 5, wherein the at least one medical device is configured to monitor signals detected by the at least one ECG sensor to identify at least one of a return to spontaneous circulation (ROSC), a cardiac arrest event, or a heart attack event in the ECG signals, and wherein the generated case file further comprises information about the ROSC, the cardiac arrest event, or the heart attack event.

7. The system of claim 6, wherein the report generated by the at least one computing device comprises the information about the ROSC, the cardiac arrest event, or the heart attack event provided by the at least one medical device.

8. The system of claim 5, wherein the at least one medical device comprises a defibrillator comprising at least one therapeutic electrode for providing cardiac therapy for the patient based on an analysis of the signals detected by the at least one ECG sensor.

9. The system of claim 1, wherein the at least one chest compression sensor comprises at least one of an accelerometer, velocity sensor, force sensor, or impedance sensor.

10. The system of claim 1, wherein the at least one chest compression sensor comprises a single axis or a multi-axis accelerometer, and wherein the accelerometer is configured to be positioned on a sternum of the patient.

11. The system of claim 10, further comprising a housing configured to be positioned on the sternum of the patient between hands of a rescuer performing the chest compressions and a chest of the patient, wherein the accelerometer is positioned in the housing.

12. The system of claim 1, wherein, to generate the case file, the at least one medical device is configured to:
receive and process the time-correlated signals from the at least one chest compression sensor,
identify and determine the times of occurrence for the plurality of the medical events represented in the time-correlated signals, and
generate the case file for the rescue effort comprising the times of occurrence for the plurality of medical events represented in the time-correlated signals

13. The system of claim 12, wherein the at least one first event comprises an end of manual chest compressions, and
the at least one second event comprises a start of automated chest compressions.

14. The system of claim 13, wherein the at least one medical device is configured to identify and determine the time of occurrence for the end of the manual chest compressions by:
generating at least one compression waveform from the received and processed time-correlated signals;
identifying portions of the at least one compression waveform representative of manual chest compressions provided for the patient; and
determining a final time of the portions of the at least one compression waveform representative of the manual chest compressions.

15. The system of claim 14, wherein the at least one medical device is configured to identify and determine the time of occurrence for the start of the automated chest compressions by:
identifying portions of the at least one compression waveform representative of automated chest compressions provided for the patient; and
determining a first time of the portions of the at least one compression waveform representative of the automated chest compressions.

16. The system of claim 14, wherein the at least one medical device is configured to identify the portions of the at least one chest compression waveform representative of manual chest compressions by:
calculating at least one chest compression parameter value for multiple segments of the at least one compression waveform;
comparing the calculated at least one chest compression parameter value for the multiple segments to a target range for the at least one chest compression parameter values representative of manual chest compressions; and
identifying segments of the multiple segments of the at least one compression waveform with the at least one chest compression parameter value within the target range.

17. The system of claim 16, wherein the at least one chest compression parameter value comprises at least one of compression rate, compression depth, compression hold time, variation in compression rate, variation in compression depth, variation in hold time, compression width, relaxation time, release time, compression average velocity, compression maximum velocity, or velocity minimum to maximum time (per chest compression cycle).

18. The system of claim 12, wherein the at least one first event comprises turning on the at least one medical device, and wherein the time of occurrence for turning on the at least one medical device is a first time recorded in the time-correlated signals, and
the at least one second event comprises a start of manual chest compressions, an end of the manual chest compressions, a start of automated chest compressions, or an end of the automated chest compressions.

19. The system of claim 1, wherein the generated case file for the rescue effort comprises the time-correlated signals received by the at least one chest compression sensor, and wherein the at least one computing device is configured to process the time-correlated signals to identify and determine the times of occurrence for the plurality of the medical events represented in the time-correlated signals.

20. The system of claim 1, wherein the at least one computing device further comprises a visual display, and wherein the at least one computing device is further configured to cause the transition time indication representative of the determined transition time to be displayed on the visual display.

21. The system of claim 1, further comprising a chest compressor configured to be positioned on a chest of the patient for providing automated chest compressions for the patient.

22. The system of claim 21, wherein the chest compressor comprises a compression belt and a belt tensioner configured to tighten the compression belt around the chest of the patient in order to compress the chest of the patient.

23. The system of claim 21, wherein the chest compressor is a piston-based device comprising:
a piston,
a piston driver,
support structures for supporting the piston and the piston driver, and
a compression pad affixed to the piston.

24. The system of claim 1, wherein the at least one computing device comprises a local portable computing device in wired or wireless communication with the at least one medical device.

25. The system of claim 1, wherein the at least one computing device is integral with and/or a component of the at least one medical device, and is configured to cause the generated report to be displayed on a display of the at least one medical device.

26. The system of claim 1, wherein the at least one computing device comprises a remote computing device or remote computer server configured to receive the case file for the rescue effort via a wired or wireless data transmission initiated from a communication device of the at least one medical device.

27. A computer-implemented method for providing transition times between types of medical treatment events provided for a patient, the method comprising:
receiving a case file comprising a time-stamped record of a plurality of events occurring during a rescue effort generated based on analysis of motion signals generated by at least one chest compression sensor;
selecting and determining a time of occurrence of at least one first event of the plurality of events from the time-stamped record;
selecting and determining a time of occurrence of at least one second event of the plurality of events from the time-stamped record occurring after the selected at least one first event;
determining a transition time between the time of occurrence of the at least one first event and the time of occurrence of the at least one second event determined from the received time-stamped record; and
generating a visual summary for the rescue effort comprising at least one transition time indication representative of the determined transition time.

28. The method of claim 27, wherein the at least one transition time is between at least one of:
(i) turning on the at least one medical device and a start of manual chest compressions,
(ii) turning on the at least one medical device and an end of manual chest compressions,
(iii) turning on the at least medical device and a start of automated chest compressions,
(iv) turning on the at least one medical device and an end of automated chest compressions,
(v) the start of the manual chest compressions and the end of the manual chest compressions,
(vi) the start of the manual chest compressions and the start of the automated chest compressions,
(vii) the start of the manual chest compressions and the end of the automated chest compressions,
(viii) the end of manual chest compressions and the start of automated chest compressions,
(ix) the end of manual chest compressions and the end of automated chest compressions, or
(x) the start of automated chest compressions and the end of automated chest compressions.

29. The method of claim 27, further comprising receiving information from at least one patient physiological sensor configured to detect signals representative of at least one patient vital sign, wherein the visual summary further comprises at least one visual indication representative of the at least one patient vital sign.

30. The method of claim 29, wherein the at least one patient vital sign comprises at least one of patient blood oxygen level, patient blood pressure, patient oxygen saturation (SPO₂), patient end-tidal CO₂, or patient heart rate.

31. The method of claim 27, further comprising receiving information about a return to spontaneous circulation (ROSC), a cardiac arrest event, or a heart attack event determined by monitoring ECG signals of the patient, wherein the visual summary further comprises at least one visual indication indicating occurrence of the ROSC, the cardiac arrest event, or the heart attack event.

32. The method of claim 27, wherein the at least one first event comprises an end of manual chest compressions, and
the at least one second event comprises a start of automated chest compressions.

33. The method of claim 27, further comprising making the visual summary available for download via a computer network, such that the visual summary is viewable by a remote computing device.

34-112. (canceled)
